(19) United States
(12) Patent Application Publication
(10) Pub. No.: US 2026/0265351 A1
(43) Pub. Date: Sep. 10, 2026

(54) HUMAN BROADLY NEUTRALIZING MONOCLONAL ANTIBODIES ASSOCIATED WITH HEPATITISC VIRUS CLEARANCE

(71) Applicant: The Johns Hopkins University, Baltimore, MD (US)

(72) Inventors: Justin R. Bailey, Timonium, MD (US); Clinton O. Ogega, Boston, MA (US)

(21) Appl. No.: 19/489,242

(22) PCT Filed: Jun. 3, 2024
(86) PCT No.: PCT/US2024/032312

§ 371 Date: Dec. 1, 2025

Related U.S. Application Data
(60) Provisional application No. 63/470,326, filed on Jun. 1, 2023.

(51) Int. Cl. C07K 16/118; A61P 31/14; C07K 14/005; C12N 15/10; C12Q 1/6806; C12Q 1/6869

(52) U.S. Cl. CPC C07K 16/118; A61P 31/14; C07K 14/005; C12N 15/1096; C12Q 1/6806; C12Q 1/6869; C07K 2317/14; C07K 2317/56; C07K 2317/76; C12N 2770/24251

(57) ABSTRACT

Broadly-neutralizing antibodies (bNAbs) associated with clearance of human hepatitis C virus (HCV) infection are provided. The bNAbs were generated against a novel mixture of full-length E2 ectodomain proteins to isolate 10,680 E2-reactive B cells from acute infection through multiple spontaneous viral clearance events.

### CROSS REFERENCE TO RELATED APPLICATIONS

This application claims the benefit of priority under 35 U.S.C. § 119 (e) to U.S. Provisional Application No. 63/470,326 filed Jun. 1, 2023, the entire contents of which is incorporated herein by reference.

### STATEMENT REGARDING FEDERALLY SPONSORED RESEARCH

This invention was made with government support under grant AI127469 awarded by the National Institutes of Health. The government has certain rights in the invention.

### BACKGROUND

Hepatitis C virus (HCV) is a single stranded, positive sense RNA virus that infects hepatocytes (Rice, 1996, Simmonds, 2004). Transmission of the virus occurs via blood-to-blood contact, with the majority of infections among people who inject drugs or in regions with unsafe medical practices (Fraser et al., 2018, Pondé, 2011). Of those infected with HCV, approximately 25% naturally clear the infection without treatment and these individuals have an 80% chance of clearing subsequent reinfections (Osburn et al., 2010). It has previously been shown that natural clearance of infection is associated with early emergence of broadly neutralizing antibodies (bNAbs) targeting the HCV envelope (E2) glycoprotein (Thomas et al., 2009, Osburn et al., 2014, Pestka et al., 2007, Raghuraman et al., 2012). It was also shown that bNAbs play a direct role in viral clearance in some individuals (Kinchen et al., 2018b). Additionally, infusion of bNAbs is protective against HCV infection in animal models (Houghton, 2011, Liang, 2013, Frey et al., 2010, de Jong et al., 2014). However, one of the challenges for vaccine development is the rich viral diversity of HCV with 8 major genotypes and more than 85 subtypes, with genomes that differ by up to 30% (Messina et al., 2015, Zein, 2000, Borgia et al., 2018, Smith et al., 2014, Martell et al., 1992, de la Peña et al., 2021). An additional limitation is the lack of a comprehensive understanding of the epitopes targeted by bNAbs mediating natural clearance of infection, and the developmental pathways giving rise to these bNAbs.

In a recent study, Weber et al. (Weber et al., 2022b) used a truncated E2 ‘core’ protein fragment to isolate B cells from four individuals who had been chronically infected with HCV for 6 to >15 years. They captured almost exclusively E2 front layer-reactive bNAbs encoded by the VH1-69 gene segment. Those data confirmed prior studies showing that V_{H}1-69 usage favors broad HCV neutralization by bNAbs (Law et al., 2008b, Bailey et al., 2017). The V_{H}1-69 gene segment is also frequently utilized by bNAbs specific for HIV and Influenza, likely because it facilitates interactions with hydrophobic domains on receptor-binding proteins (Weber et al., 2022b, Chan et al., 2001, Tzarum et al., 2019, Chen et al., 2019, Kumar et al., 2020, Lang et al., 2017, Lingwood et al., 2012, Smith et al., 2019). In addition to the V_{H}1-69 gene segment, several human anti-HCV bNAbs also use D2-15, which encodes two cysteine residues that form a disulfide motif at the tip of CDRH3 (Flyak et al., 2018, Tzarum et al., 2019). Due to the limited number of bNAbs isolated to date, the extent to which V_{H}1-69 or D2-15 gene usage is required for broad HCV neutralization has remained unclear, and it is not known whether these V and D-genes also predominate in bNAbs of persons capable of natural HCV clearance. These are critical questions for vaccine development, since exclusive reliance on single V and D-gene segments would restrict the genetic pathways available for bNAb induction by vaccines, while also limiting the value of most animal models, including mice, for vaccine development, since they do not encode IGHV1-69 or IGD2-15 orthologs.

### SUMMARY

Embodiments are directed to broadly neutralizing antibodies (bNAbs) and bNAb-inducing vaccines that protects against persistent HCV infection.

Accordingly, in certain aspects, a method of producing neutralizing antibodies to hepatitis virus comprises isolating antigenically diverse proteins from the hepatitis virus, incubating peripheral blood mononuclear cells (PBMCs) with a mixture of the isolated antigenic diverse proteins, sorting the PBMCs to obtain antigen reactive class-switched memory B cells and non antigen reactive class-switched memory B cells, seeding the antigen reactive class-switched memory B cell cells in limiting dilutions in cell culture wells wherein each cell culture well comprises at least one antigen reactive class-switched memory B cell, stimulating the class-switched memory B cell cultures with one or more cytokines and antigen, isolating supernatants from the antigen reactive class-switched memory B cell cultures, and, producing neutralizing antibodies to hepatitis virus. In certain embodiments, In certain embodiments, the hepatitis virus comprises hepatitis A virus (HAV), hepatitis B virus (HBV), hepatitis C virus (HCV), hepatitis D virus (HDV), or hepatitis E virus (HEV). In certain embodiments, the hepatitis virus is HCV. In certain embodiments, the antigenically diverse proteins isolated from the HCV comprise E2 ectodomain proteins. In certain embodiments, the antigenically diverse proteins E2 ectodomain proteins are isolated from one or more HCV genotypes comprising genotypes 1-7 or 8. In certain embodiments, the HCV antigenically diverse E2 ectodomain proteins are isolated from one or more HCV subtypes. In certain embodiments, the one or more HCV subtypes comprise subtypes comprise subtypes 1a-1o, 2a-2v, 3a-3k, 4a-4w, 5a, 6a-6xj, 7a-7b or 8a. In certain embodiments, the E2 ectoproteins are isolated from subtype 1. In certain embodiments, E2 ectoproteins are isolated from subtypes 1a, subtype 1b or the combination thereof. In certain embodiments, the nucleic acids encoding immunoglobulin variable heavy and light chain sequences isolated from antigen reactive class-switched memory B cells are subjected to RT-PCR and sequenced. In certain embodiments, the immunoglobulin variable sequences are cloned into human IgG kappa (κ) or lambda (λ) expression plasmids. In certain embodiments, the heavy and light chains are co-expressed to generate monoclonal antibodies. In certain embodiments, the monoclonal antibodies comprise immunoglobulin G (IgG) isotypes IgG1, IgG2, IgG3 or IgG4. In certain embodiments, the IgG isotype is IgG1. In certain embodiments, the antibody variable heavy chain gene segments comprise: V_{H}1-69, V_{H}1-18, V_{H}1-46, V_{H}1-3, V_{H}3-20, V_{H}3-23, V_{H}4-34, V_{H}3-74, V_{H}3-48, V_{H}3-74, or V_{H}4-30. In certain embodiments, the antibody D gene segments comprise D2-2, D2-15, D3-10, D4-17, or D6-13. In certain embodiments, the antibody comprises a complementarity-determining region 1 (CDRH1) sequence, a complementarity-determining region 3 (CDRH3) sequence, or the combination thereof. In certain embodiments, the CDRH1 sequence and/or the CDRH3 sequence comprise one or more amino acid mutations, deletions, substitutions, insertions or combinations thereof.

In another aspect, an isolated nucleic acid comprises (i) a variable heavy chain having at least a 90% sequence identity to SEQ ID NOs: 1-57 or 58; (ii) a variable kappa light (Vκ) chain having at least a 90% sequence identity to SEQ ID NOs: 59-107 or 108; or (iii) a variable lambda light (Vλ) chain having at least a 90% sequence identity to SEQ ID NOs: 109-122 or 123, or combinations thereof. In certain embodiments, (i) the variable heavy chain (V_{H}) comprises SEQ ID NOs: 1-57 or 58; (ii) the variable kappa light (Vκ) chain comprises SEQ ID NOs: 59-107 or 108; or (iii) the variable lambda light (Vλ) chain comprises SEQ ID NOs: 109-122 or 123, or combinations thereof. In certain embodiments, the V_{H} chain comprises one or more mutations, deletions, insertions, substitutions, variants or combinations thereof. In certain embodiments, the Vκ chain comprises one or more mutations, deletions, insertions, substitutions, variants or combinations thereof. In certain embodiments, the Vλ chain comprises one or more mutations, deletions, insertions, substitutions, variants or combinations thereof. In certain embodiments, the isolated nucleic acid comprises any one Vλ sequence comprising SEQ ID NOs: 1-57 or 58 and any one Vκ sequence comprising SEQ ID NOs: 59-107 or 108. In certain embodiments, the isolated nucleic acid comprises any one V_{H} sequence comprising SEQ ID NOs: 1-57 or 58 and any one Vλ sequence comprising SEQ ID NOs: 109-122 or 123.

In another aspect, an isolated variable heavy chain (V_{H}) comprises SEQ ID NOs: 1-57 or 58.

In another aspect, an isolated variable kappa light (Vκ) chain comprising SEQ ID NOs: 59-107 or 108.

In another aspect, an isolated variable lambda light (Vλ) chain comprises SEQ ID NOs: 109-122 or 123.

In another aspect, a method of preventing or treating a hepatitis C virus (HCV) infection comprises administering to a subject a therapeutically effective amount of an HCV neutralizing antibody, thereby, treating the subject. In certain embodiments, the HCV neutralizing antibody comprises a variable heavy chain (V_{H}) comprising SEQ ID NOs: 1-57 or 58 and a variable kappa light (Vκ) chain comprising SEQ ID NOs: 59-107 or 108. In certain embodiments, the HCV neutralizing antibody comprises a variable heavy chain (V_{H}) comprising SEQ ID NOs: 1-57 or 58 and a variable lambda light (Vλ) chain comprising SEQ ID NOs: 109-122 or 123. In certain embodiments, the HCV neutralizing antibodies are administered to the subject in combination with one or more anti-viral agents.

In another aspect, an isolated nucleic acid comprises: (i) a variable heavy chain encoded by a nucleic acid sequence having at least a 90% sequence identity to SEQ ID NOs: 1-57 or 58; or (ii) a variable kappa light (Vκ) chain encoded by a nucleic acid sequence having at least a 90% sequence identity to SEQ ID NOs: 59-107 or 108; or (iii) a variable lambda light (Vλ) chain encoded by a nucleic acid sequence having at least a 90% sequence identity to SEQ ID NOs: 109-122 or 123, or combinations thereof. In certain embodiments, the isolated nucleic acid comprises (i) the variable heavy chain (V_{H}) encoded by a nucleic acid sequence comprising SEQ ID NOs: 1-57 or 58; or (ii) the variable kappa light (Vκ) chain encoded by a nucleic acid sequence comprising SEQ ID NOs: 59-107 or 108; or (iii) the variable lambda light (Vλ) chain encoded by a nucleic acid sequence comprising SEQ ID NOs: 109-122 or 123, or combinations thereof. In certain embodiments, the nucleic acid sequence encodes an amino acid sequence comprising one or more amino acid mutations comprising Y29F, 130S/T, R100aS, R100bG/E, Y100fA or combinations thereof.

In another aspect, an expression vector comprises: (i) a variable heavy chain sequence having at least a 90% sequence identity to SEQ ID NOs: 1-57 or 58; or (ii) a variable kappa light (Vκ) chain sequence having at least a 90% sequence identity to SEQ ID NOs: 59-107 or 108; or (iii) a variable lambda light (Vλ) chain sequence having at least a 90% sequence identity to SEQ ID NOs: 109-122 or 123, or combinations thereof. In certain embodiments, the expression vector comprises (i) the variable heavy chain (V_{H}) sequence comprising SEQ ID NOs: 1-57 or 58; (ii) the variable kappa light (Vκ) chain sequence comprising SEQ ID NOs: 59-107 or 108; or (iii) the variable lambda light (Vλ) chain sequence comprises SEQ ID NOs: 109-122 or 123, or combinations thereof. In certain embodiments, the expression vector comprises any one V_{H} sequence comprising SEQ ID NOs: 1-57 or 58 and any one Vκ sequence comprising SEQ ID NOs: 59-107 or 108. In certain embodiments, the expression vector comprises any one V_{H} sequence comprising SEQ ID NOs: 1-57 or 58 and any one Vλ sequence comprising SEQ ID NOs: 109-122 or 123.

In another aspect, an expression vector comprises one or more nucleic acid sequences comprising SEQ ID NOs: 1-57 or 58.

In another aspect, an expression vector comprises one or more nucleic acid sequences comprising SEQ ID NOs: 59-107 or 108.

In another aspect, an expression vector comprises one or more nucleic acid sequences comprising SEQ ID NOs: 109-122 or 123.

In another aspect, a host cell comprises an isolated nucleic acid sequence embodied herein.

In another aspect, a host cell comprises an expression vector embodied herein.

### Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. Definitions of common terms can be found in Singleton et al., Dictionary of Microbiology and Molecular Biology 3rd ed., J. Wiley & Sons New York, NY (2001); March, Advanced Organic Chemistry Reactions, Mechanisms and Structure 5th ed., J. Wiley & Sons New York, NY (2001); Michael Richard Green and Joseph Sambrook, Molecular Cloning: A Laboratory Manual, 4th ed., Cold Spring Harbor Laboratory Press, Cold Spring Harbor, N.Y., USA (2012); Davis et al., Basic Methods in Molecular Biology, Elsevier Science Publishing, Inc., New York, USA (2012); Jon Lorsch (ed.) Laboratory Methods in Enzymology: DNA, Elsevier, (2013); Frederick M. Ausubel (ed.), Current Protocols in Molecular Biology (CPMB), John Wiley and Sons, (2014); John E. Coligan (ed.), Current Protocols in Protein Science (CPPS), John Wiley and Sons, Inc., (2005); and Ethan M Shevach, Warren Strobe, (eds.) Current Protocols in Immunology (CPI) (John E. Coligan, ADA M Kruisbeek, David H Margulies, John Wiley and Sons, Inc., (2003); each of which provide one skilled in the art with a general guide to many of the terms used in the present application.

Standard nomenclature is used for the natural amino acids and their abbreviations. For example, L-alanine is represented with the three-letter abbreviation Ala, or one-letter abbreviation “A”. Where indicated, the “D” stereoisomer of alanine is represented as D-Ala.

Standard nomenclature is used for the bases of DNA, with cytosine, guanosine, adenine, and thymine indicated as “C”, “G”, “A”, and “T”, and codons that encode DNA follow the standard genetic code, for example the amino acid Leu is encoded by TTA, TTG, CTT, CTC, CTA or CTG, and Asp is encoded by GAT or GAC.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude, preferably within 5-fold, and also preferably within 2-fold, of a value. Where particular values are described in the application and claims, unless otherwise stated the term “about” meaning within an acceptable error range for the particular value should be assumed. All numeric values are herein assumed to be modified by the term “about”, whether or not explicitly indicated. The recitation of numerical ranges by endpoints includes all numbers within that range (e.g., 1 to 5 includes 1, 1.5, 2, 2.75, 3, 3.80, 4, and 5).

In the description and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C”, “one or more of A, B, and C” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used herein, the term “affinity” is meant as a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, including use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

The term “amino acid” as used herein refers to naturally occurring and synthetic α, β, γ, and δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. The amino acids can be non-naturally occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine. When the term amino acid is used, it is considered to be a specific and independent disclosure of each of the esters of α, β, γ, and δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)₂, and Fv). The term “immunoglobulin” (Ig) is used interchangeably with “antibody” herein. The basic 4-chain antibody unit is a heterotetrameric glycoprotein composed of two identical light (L) chains and two identical heavy (H) chains. An antibody usually has both variable and constant regions whereby the variable regions are mostly responsible for determining the specificity of the antibody and will comprise complementarity determining regions (CDRs). An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the CL is aligned with the first constant domain of the heavy chain (C_{H1}). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Ten and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1 and IgA2. The terms “full-length antibody,” “intact antibody” or “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)2 and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H1}). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H1} domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known. The Fc fragment comprises the carboxy-terminal portions of both H chains held together by disulfides. The effector functions of antibodies are determined by sequences in the Fc region, the region which is also recognized by Fc receptors (FcR) found on certain types of cells.

The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, gene-editing agents, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof. The term also refers to non-nucleoside reverse transcriptase inhibitors (NNRTIs), nucleoside reverse transcriptase inhibitors (NRTIs), analogs, variants etc.

The term “bispecific” as provided herein is used according to its conventional meaning well known in the art and refers to a bispecific recombinant protein capable of simultaneously binding to two different antigens. In contrast to traditional monoclonal antibodies, bispecific antibodies consist of two independently different antibody regions (e.g., two single-chain variable fragments (scFv)), each of which binds a different antigen.

As used herein “broadly neutralizing antibodies (bnAbs)”, refer to antibodies capable of neutralizing the majority of strains of a given highly antigenically variable pathogen. In certain embodiments, the pathogen is a virus. In certain embodiments, the pathogen is hepatitis C virus (HCV).

The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

The term “complementarity determining region” or “CDR” refers to variable regions of either H (heavy) or L (light) chains (also abbreviated as VH and VL, respectively) and contains the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have 3 CDR regions, each non-contiguous with the others (termed L1, L2, L3, H1, H2, H3) for the respective light (L) and heavy (H) chains. The accepted CDR regions have been described by Kabat et al., Sequences of Proteins of Immunological Interest, 5th Ed. Public Health Service, National Institutes of Health, Bethesda, Md. (1991).

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) can recognize and bind antigen, although at a lower affinity than the entire binding site.

The term “hinge” or “hinge region” refers to a flexible connector region, e.g. natural or synthetic polypeptides, or any other type of molecule, providing structural flexibility and spacing to flanking polypeptide regions.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit, or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Am. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol., 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996). A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops and are used by Oxford Molecular's AbM antibody modeling software. The “contact” HVRs are based on an analysis of the available complex crystal structures. The residues from each of these HVRs are noted below.

The term “linker”, also referred to as a “spacer” or “spacer domain” as used herein, refers to an amino acid or sequence of amino acids that that is optionally located between two amino acid sequences in a fusion protein of the invention.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

As used herein, a “natural amino acid” refers to the twenty genetically encoded alpha-amino acids. See, e.g., Biochemistry by L. Stryer, 3^{rd} ed. 1988, Freeman and Company, New York for structures of the twenty natural amino acids.

“Neutralization” of viruses, in particular HCV, is defined here as the abrogation of virus infectivity in vitro by the binding of a neutralizing compound to the virion. Thus, the target of the neutralizing compound does not have to be of virus origin, as long as it is present on the virion. The definition does not include the block of infection by a neutralizing compound that binds to a receptor for the virus on the (host) cell surface. It is reasonable to add a further criterion: that neutralizing compounds act before the first major biosynthetic event in the virus replicative cycle has taken place. Then, it is a matter for experimental investigation whether neutralization can block a step between virus entry and that later event. According to this criterion, interference with release of progeny virus should not be termed neutralization (adapted from Kiasse and Sattentau, 2002).

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

“Optional” or “optionally” means that the subsequently described event or circumstance can or cannot occur, and that the description includes instances where the event or circumstance occurs and instances where it does not.

As used in this specification and the appended claims, the term “or” is generally employed in its sense including “and/or” unless the content clearly dictates otherwise and should be understood to mean “either or both” of the elements so conjoined, e.g., elements that are conjunctively present in some cases and disjunctively present in other cases.

“Parenteral” administration of an immunogenic composition includes, e.g., subcutaneous (s.c.), intravenous (i.v.), intramuscular (i.m.), intravitreal (i.v.i), intracisterna magna (i.e.m), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A polynucleotide is typically composed of a specific sequence of four nucleotide bases: adenine (A), cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994). “Functional fragments” of the antibodies of the disclosure comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

As used herein, an “unnatural amino acid,” “non-natural”, “modified amino acid” or “chemically modified amino acid” refers to any amino acid, modified amino acid, or amino acid analogue other than the twenty genetically encoded alpha-amino acids. Unnatural amino acids have side chain groups that distinguish them from the natural amino acids, although unnatural amino acids can be naturally occurring compounds other than the twenty proteinogenic alpha-amino acids. In addition to side chain groups that distinguish them from the natural amino acids, unnatural amino acids may have an extended backbone such as beta-amino acids.

Non-limiting examples of non-natural amino acids include selenocysteine, pyrrolysine, homocysteine, an O-methyl-L-tyrosine, an L-3-(2-naphthyl) alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline. In an embodiment of the helicases described herein, one or more amino acids of the helicase are substituted with one or more unnatural amino acids and/or one or more natural amino acids.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

As used herein, the term “virus” includes any type of virus or virus vector. For example, adenovirus, adeno-associated virus (AAV), recombinant adeno-associated virus (rAAV), herpes simplex virus, lentivirus, gammaretrovirus, retrovirus, alphavirus, flavivirus, rhabdovirus, measles virus, Newcastle disease virus, poxvirus, vaccinia virus, modified Ankara virus, vesicular stomatitis virus, picornavirus. In various embodiments the virus is a chimeric virus, a synthetic virus, a recombinant virus, a mosaic virus or a pseudotyped virus.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In preferred embodiments, the genes, nucleic acid sequences, amino acid sequences, peptides, polypeptides and proteins are human. The term “gene” is also intended to include variants.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.), Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N. Y); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D show a selection of an Elite Neutralizer for BCR repertoire analysis. FIG. 1A: Neutralizing breadth of early infection plasma from 63 BBAASH participants with either spontaneous clearance of infection (n=21, prefix “C”) or persistence of infection (n=42, prefix “P”). The 10% of participants with the greatest neutralizing breadth (n=6) were designated Elite Neutralizers (EN). FIG. 1B: Neutralizing activity of 6 EN against each of 17 Tier 1 (sensitive) to Tier 4 (resistant) HCVpp. Values for subject C110 are indicated in red. Error bars indicate standard deviations. FIG. 1C: Viral RNA levels of subject C110 demonstrating spontaneous clearance of 3 infections (delineated by distinct colors). The timepoint used for mAb isolation (D2178) is indicated (arrow) along with the four timepoints used for BCR-seq analysis (red dots). FIG. 1D: C110 plasma neutralizing breadth at the 5 timepoints used for BCR-seq and mAb analysis. Neutralization was measured in duplicate in one experiment.

FIGS. 2A-2G demonstrate the monoclonal antibody (Mab) isolation and BCR sequence analysis. FIG. 2A: E2-reactive B cell staining with a mixture of three antigenically distinct soluble E2 proteins. FIG. 2B: B cells cultured at varying cell density, with frequency (%) of wells positive for human IgG or reactive with each E2 variant. FIG. 2C: Frequency (%) of IgG positive 2 or 1 B cell supernatants reactive with each E2 variant or cross-reactive with multiple E2 variants. FIG. 2D: CDRH3 lengths of E2-nonreactive or longitudinal E2-reactive BCRs or mAbs. FIG. 2E: CDRL3 lengths of E2-nonreactive or longitudinal E2-reactive BCRs or mAbs. FIG. 2F: V_{H}-gene nucleotide identity to inferred germline sequences. FIG. 2G: V_{K} or V_{L}-gene nucleotide identity to inferred germline sequences. For D-E, distribution is shown, with boxes indicating the 25^{th} percentile (lower border), 75^{th} percentile (upper border), and median (horizontal line). For F-G, boxes indicate 25^{th} and 75^{th} percentiles, whiskers indicate maximum and minimum values that fall within 1.5× the interquartile range, horizontal lines are medians, ****p<0.0001; ***p<0.001, **p<0.01, *p<0.05.

FIGS. 3A-3F demonstrate that V_{H}1-69 and D2-2/D2-15 usage favor broad neutralizing activity but are not required. Frequency of usage of V_{H} (FIG. 3A) or V_{K}/V_{L} (FIG. 3B) gene segments among E2-nonreactive or E2-reactive BCRs. Only V-genes used by at least one mAb are shown here, with the remainder in FIGS. 9A and 9B. *p<0.05 adjusted for multiple comparisons by the Benjamini-Hochberg method. FIG. 3C: Neutralizing breadth of 55 E2-reactive mAbs measured using the panel of 17 HCVpp. FIG. 3D: Neutralizing breadth of mAbs (# of 17 HCVpp neutralized) segregated by V_{H}-gene usage. Each point represents one mAb. FIG. 3E: Neutralizing breadth of mAbs (# of 17 HCVpp neutralized) segregated by D-gene usage. FIG. 3F: Neutralizing breadth of mAbs using both V_{H}1-69 and D2-2/D2-15, either, or neither. In FIGS. 3D-3F, dashed line indicates neutralization of 9 HCVpp, the threshold for bNAb designation. Solid horizontal lines indicate medians. *p<0.05, adjusted for multiple comparisons. Neutralization values are the average of two independent experiments performed in duplicate.

FIG. 4 demonstrates that HCV bNAbs target conserved front layer and novel epitopes on the E2 protein. bNAbs were segregated based on >50% loss of binding (highlighted in red) to E2 (1a157 variant) with multiple mutations in the front layer (FRLY KO) or E2 (1a53 variant) with multiple mutations in the AS412 epitope (AS412 KO), relative to binding to wild-type E2 protein. E1E2 (H77 variant) binding competition was also measured between each mAb and back-layer-reactive reference mAb ARIA (>50% inhibition of binding due to ARIA competition highlighted in red). FRLY or non-FRLY mAbs were grouped by hierarchical clustering based on relative binding to a panel of 25 single alanine mutant E1E2 proteins, relative to binding to wild-type protein. 12 reference mAbs (bold) with known binding epitopes were included as controls. Neutralizing breadth (# of 17 HCVpp neutralized) of each mAb is indicated.

FIGS. 5A-5H show that crystal structures of hcab55-E2ecto, hcab64-E2ecto, and hcab40-E2ecto complexes reveal neutralizing epitopes of non-V_{H}1-69 HCV bNAbs. FIG. 5A: Crystal structures of the hcab55-E2ecto, hcab64-E2ecto, and hcab40-E2ecto complexes. E2 ectodomains are shown as gray cartoon representations with N-glycans highlighted as sticks and disulfide bonds shown as yellow sticks. Fabs are shown as cartoons with hcab55-E2ecto colored in red, hcab64-E2ecto colored in orange, and hcab40-E2ecto colored in green. The crystal structures were superimposed on E2ecto proteins. FIG. 5B: Comparison of buried surface areas (BSAs). FIG. 5C: Superposition of hcab55-E2ecto, hcab64-E2ecto, hcab40-E2ecto, HEPC74-E2ecto (PDB 6MEH), and HEPC46-E2ecto (PDB 6MEJ) complex structures. Structures were superimposed on the E2 and are shown as cartoon representations with E2 colored in gray, hcab55-Fab colored in red, hcab64-Fab colored in orange, hcab40-Fab colored in green, HEPC74-Fab colored in purple, and HEPC46-Fab colored in cyan. Disulfide bonds are shown as yellow sticks. The HEPC3-Fab in the HEPC46-E2ecto-HEPC3 structure (PDB 6MEJ) is not shown for clarity. FIG. 5D: Heavy-chain CDR (CDRH) loops of hcab55, hcab64, hcab40, and HEPC74 (PDB 6MEH) mapped onto the E2 surface. The crystal structures were superimposed on E2ecto proteins. HC interacting residues are colored in dark gray on the E2 surface. Epitopes on the E2 front layer (surface representation) were defined as residues in E2 containing an atom within 4 Å of the bound Fab. The location of α1-helix, α2-helix, ß11, 12-strands, loop 586-591, and loop 603-607 in E2 are indicated by black cartoon representations, and the C429-C503 disulfide bond is indicated by yellow sticks. FIG. 5E: Comparison of the CDRH loop positions in E2 complex structures with hcab55, hcab64, hcab40, and HEPC74. E2ecto structures are shown as surface representation and CDRHs as tubes. CDRH loops are colored as in (FIG. 5D), and important features of E2ecto and the position of CDRH3 are indicated. E2 surfaces are colored by structural components: AS412, dark green; front layer, light green; VR2, red; β sandwich, violet; CD81bl, blue; VR3, gray; back layer, yellow. FIG. 5F: Interactions of Hcab40-Fab CDRH1 loop with E2ecto. Potential hydrogen bonds are shown as black dashed lines, and residues at the interface are indicated. Hydrogen bonds for the 3.1 Å hcab40-Fab-E2ecto structure should be considered tentative. FIG. 5G: Interactions of hcab40-Fab residues S74 and R74 (FR3 region) with E2ecto. Residues at the interface are indicated. FIG. 5H: Interactions of hcab40-Fab CDRH3 loop with E2ecto. Residues at the interface and the C607-C644 disulfide bond in the back layer of E2 are indicated.

FIGS. 6A, 6B demonstrate the convergent evolution of FRLY bNAbs using diverse V_{H} and D-gene segments. FIG. 6A: CDRH1-2 substitutions shared by multiple FRLY bNAbs and significantly enriched (bold) relative to E2-nonreactive BCRs from the same participant. E2-nonreactive BCRs were matched to FRLY bNAbs based on V-gene usage and % homology to germline sequences. FIG. 6B: CDRH3 amino acids shared by multiple FRLY bNAbs and significantly enriched (bold) relative to E2-nonreactive BCRs. E2-nonreactive BCRs were matched to FRLY bNAbs based on CDRH3 length. Germline-encoded amino acids are in green, somatic mutation-encoded substitutions are in pink, and junctional nucleotide-encoded amino acids are in blue. P values were determined using Fischer's exact test, adjusted for multiple comparisons using false discovery rate. Hcab35 was excluded from the CDRH3 analysis because extreme length made alignment difficult. Numbering according to the Kabat method.

FIGS. 7A-7F demonstrate that bNAb substitutions arising by convergent evolution enhance neutralizing breadth and potency. FIGS. 7A-7E: Neutralizing breadth across a panel of 17 HCVpp by wild-type (WT) front-layer-reactive hcabs or hcabs with a single amino acid altered by site-directed mutagenesis. Each pair of linked points represents a single HCVpp. Values are the average of two independent experiments, tested in duplicate. For each mAb pair, HCVpp that were not neutralized by either WT or reverted mAbs were excluded from the analysis. FIG. 7A: Y29F reversion. FIG. 7B: I30S/T reversion. FIG. 7C: R100aS reversion. FIG. 7D: R100bG/E substitution. FIG. 7E: Y100fA substitution. Significance determined by Wilcoxon paired signed rank test. NS=not significant, *p<0.05, **p<0.01, ***p<0.001. FIG. 7F: Neutralizing potency against heterologous HCVpp by wild type (WT) hcabs or hcabs with a single amino acid altered by site-directed mutagenesis. IC₅₀ values were calculated from curves fit to neutralization tests performed with serial mAb dilutions (shown in FIGS. 10A-10E). The HCVpp used for each test is indicated.

FIG. 8 shows the gating schematic of sorted E2-reactive and nonreactive mature class-switched B cells. PBMC from healthy HCV(−) and longitudinal timepoints of Elite Neutralizer, subject C110, were stained with a mixture of 3 antigenically diverse subtype 1a and 1b soluble E2 ectodomain proteins (1b21, 1a157, and 1b09) as antigen baits to capture and sort single E2-reactive and non-reactive mature class-switched B cells (CD19+, CD10−, CD3−, IgM−, IgD−, E2+/−).

FIGS. 9A, 9B show the frequency of usage of V_{H} (FIG. 9A) or V_{K}/V_{L} (FIG. 9B) gene segments among E2-nonreactive BCRs, E2-reactive BCRs, or mAbs.

FIGS. 10A-10E demonstrate the neutralizing potency against heterologous HCVpp by wild type (WT) FRLY hcabs or hcabs with a single amino acid altered by site-directed mutagenesis. Graphs are representative of two independent experiments performed in duplicate. Points are means and error bars represent standard deviations.

FIG. 11 demonstrates the longitudinal evolution of FRLY bNAbs. Highlighter plots showing substitutions appearing in BCRs of E2-reactive B cells or bNAbs isolated at longitudinal timepoints. BCRs in the lineage of each bNAb were identified in the BCR-seq dataset based on shared V_{H} and J_{H} gene segment usage and identical CDRH3 length. The inferred germline sequence of each bNAb was determined using IgBlast. Vertical lines indicate positions with changes relative to the master sequence (m), which was the germline sequence of each bNAb lineage. Representative CDRH1-3 amino acids conferring enhanced neutralizing activity and enriched across bNAbs (from FIGS. 6A-6B) are indicated. Amino acid sequences were aligned in Mega.

FIG. 12 (includes FIGS. 12A-12B): HCV bNAbs target at least three major antigenic sites on the E2 protein. FIG. 12A: bNAbs were segregated based on >50% loss of binding to E2 with multiple mutations in the front layer (FRLY KO) or in the AS412 epitope (AS412 KO), relative to binding to wild-type E2 protein. Six reference mAbs (bold) with known binding epitopes as well as previously characterized C110 bNAb HEPC74 were included as controls. Curves were fit, and area under the curve (AUC) was calculated from binding of 5-fold serial dilutions of each mAb, performed in duplicate. FIG. 12B: Data from bio-layer interferometry epitope binning experiments using 17 hcabs and 5 reference antibodies (italicized). Numbers indicate the percentage binding of second mAb in the presence of the first mAb compared with binding of un-competed second mAb. mAbs were judged to compete for the same antigenic site if the maximum binding of the second mAb was reduced to <30% of its un-competed binding (black boxes with white numbers). The mAbs were considered non-competing if the maximum binding of the second mAb was >60% of its un-competed binding (white boxes with red numbers). Gray boxes with black numbers indicate an intermediate phenotype (competition resulted in between 30% and 60% of un-competed binding). Dashed lines in red (contains FRLY reference mAbs), dark blue (contains a b sheet reference mAb), and green (contains no reference mAbs) indicate three major competition groups. The light blue dashed line indicates a minor antigenic group that partially overlaps with the FRLY and b sheet antigenic groups. A reference bNAb HCV1 is a single member of the AS412 competition group, indicated by yellow dashed lines.

FIG. 13: (includes 13A-13E): HCV FRLY bNAbs display convergent evolution. (FIG. 13A) CDRH1-2 substitutions shared by multiple FRLY bNAbs and significantly enriched (bold) relative to E2-nonreactive BCRs from the same participant. E2-nonreactive BCRs were matched to FRLY bNAbs based on V-gene usage and % homology to germline sequences. (FIG. 13A-C) CDRH3 amino acids shared by multiple FRLY bNAbs (A), b sheet mAbs (B), or back layer bNAbs (FIG. 13C) and significantly enriched (bold) relative to E2-nonreactive BCRs. E2-nonreactive BCRs were matched to FRLY, b sheet, or back layer hcabs based on V-gene usage, % homology to germ line, and CDRH3 length. Germline-encoded amino acids are in green, somatic mutation-encoded substitutions are in pink, and junctional nucleotide-encoded amino acids are in blue. p values were determined using Fischer's exact test, adjusted for multiple comparisons using the Benjamini-Hochberg correction. Numbering according to the Kabat method. (FIG. 13D) Neutralizing breadth across a panel of 17 HCVpp by wild-type (WT) front-layer-reactive hcabs or hcabs with a single amino acid altered by site-directed mutagenesis. Each pair of linked points represents a single HCVpp. Values are the average of two independent experiments, tested in duplicate. For each mAb pair, HCVpp that were not neutralized by either WT or mutant mAbs were excluded from the analysis. Significance determined by Wilcoxon paired signed rank test. NS, not significant, *p<0.05, **p<0.01, ***p<0.001. (FIG. 13E) Neutralizing potency against heterologous HCVpp by wild-type (WT) hcabs or hcabs with a single amino acid altered by site-directed mutagenesis. The HCVpp used for each test is indicated. Graphs are representative of two independent experiments performed in duplicate. Points are means, and error bars represent standard deviations.

FIG. 14 (includes FIGS. 14A-14B, and refers to FIG. 3): H1-69 was enriched among E2-reactive BCRs at only one timepoint. Frequency of usage of VH (FIG. 14A) or VK/VL (FIG. 14B) gene segments among E2-nonreactive BCRs, E2-reactive BCRs, or mAbs. The subset of these data for all VH or VK/Lgenes used by isolated mAbs is shown in FIG. 3.

FIG. 15 (refers to FIG. 3). Broad neutralization is not predominantly driven by binding affinity. Binding EC50 values of bNAbs and non-bNAbs measured with 1a157, 1b09, and 1b21 E2 glycoproteins. Binding of serial dilutions of each hcab was measured in duplicate by ELISA. Curves were used to calculate 50% effective concentration (EC50) values. Horizontal lines indicate medians. Statistical significance was calculated using Mann-Whitney test. NS=not significant, *p<0.05.

FIG. 16 (includes FIGS. 16A-16D, refers to FIG. 5): Structural analysis reveals conserved bNAb-E2 interactions. (FIGS. 16A-D) Interactions between the E2 and key residues in hcab55 (FIG. 16A), hcab64 (FIG. 16B), hcab40 (FIG. 16C), and hcab 17 (FIG. 16D) Fabs. On the left, HC interacting residues are colored in dark gray on the E2 surface, and CDRH loops of hcab55, hcab64, hcab40, and hcab 17 are shown and colored as in FIG. 6. Potential hydrogen bonds are shown as black dashed lines, salt bridges as red dashed lines, and residues at the interface are indicated. A potential hydrogen bond or salt bridge between two atoms is indicated by an orange dashed line. Hydrogen bonds and salt bridges for the 3.1 Å hcab40-Fab-E2 and 3.3 Å hcab 17-Fab-E2 structures should be considered tentative.

FIG. 17 (includes FIGS. 17A-17B, refer to FIG. 5): The hcab40 epitope in the back layer of E2 overlaps with AR4A epitope in E1E2. (FIG. 17A) The AR4A Fab recognizes the E2 base region near the interface with the E1. The cryo-EM structure of HCV E1E2 heterodimer in complex with AR4A-Fab (PDB 7T6X) is shown as a cartoon representation, and E1E2 disulfide bonds are shown as yellow sticks. AR4AVHVL is colored in red, E2 head domain is colored in black, E2 base is colored in light gray, E2 stem is colored in light blue, and E1 is colored in wheat. For clarity, the AR12009-Fab and IGH505-Fab in the AR4A-E1E2-AR12009-IGH505 structure are not shown for clarity. The inset highlights the AR4A CDRH3 interactions with the E2 base. (FIG. 17B) The model of E1E2 interactions with hcab40 demonstrates that hcab40 would clash with the E2 base region. The model was generated by aligning the hcab40-E2 structure with the AR4A-E1E2 structure (PDB 7T6X) on E2 Ca atoms. Steric clashes are indicated by a red star. An inset highlights the steric clashes between hcab40 CDRH3 and E2 base region.

### DETAILED DESCRIPTION

Broadly-neutralizing antibodies (bNAbs) are associated with natural clearance of human hepatitis C virus (HCV) infection, informing vaccine development. To date, most HCV-reactive human bNAbs target the envelope glycoprotein 2 (E2) front-layer and utilize the V_{H}1-69 gene segment, suggesting that bNAb induction is genetically restricted. Here, we performed longitudinal B cell receptor (BCR) repertoire analysis on an Elite Neutralizer, using a novel mixture of full-length E2 ectodomain proteins to isolate 10,680 E2-reactive B cells from acute infection through multiple spontaneous viral clearance events. We performed BCR sequencing, characterized monoclonal B cell cultures, and isolated bNAbs. Unexpectedly, many bNAbs targeted novel E2 epitopes, and the majority did not utilize V_{H}1-69. Multiple front-layer-reactive bNAb lineages using distinct V_{H} and D-genes showed early acquisition of the same breadth-enhancing somatic mutations, including hydrogen-bond-forming contacts in bNAb-E2 structures. These findings reveal convergent bNAb-generating evolutionary pathways and conserved clearance-associated bNAb-E2 interactions, creating a roadmap for vaccine development.

### Hepatitis C Virus (HCV)

Hepatitis C virus is an enveloped, positive-strand, RNA virus classified within the Hepacivirus genus, one of the four genera of the Flaviviridae virus family. The HCV particles consist of a nucleocapsid containing the viral genome surrounded by an endoplasmic reticulum-derived membrane crowned by the E1-E2 envelope proteins. It was suggested that the HCV particle is a hybrid lipoviral particle (Bartenschlager R, et al., Assembly of infectious hepatitis C virus particles. Trends Microbiol (2011) 19(2):95-103. doi: 10.1016/j.tim.2010.11.005) that incorporates a thick shell of host-derived apolipoproteins coating the viral surface and may reduce virus sensitivity to neutralizing antibodies (NAbs) (Catanese M T, et al. Ultrastructural analysis of hepatitis C virus particles. Proc Natl Acad Sci USA (2013) 110(23):9505-10. doi: 10.1073/pnas. 1307527110; Fauvelle C, et al. Apolipoprotein E mediates evasion from hepatitis C virus neutralizing antibodies. Gastroenterology (2016) 150(1):206-17.e4. doi: 10.1053/j.gastro.2015.09.014; Bankwitz D et al. Maturation of secreted HCV particles by incorporation of secreted ApoE protects from antibodies by enhancing infectivity. J Hepatol (2017) 67(3): 480-9. doi: 10.1016/j.jhep.2017.04.010). This unique coating of the HCV virion is structurally distinct from other members of the Flaviviridae family. The E1 and E2 are type I transmembrane glycoproteins with C-terminal transmembrane domains that form a heterodimer on the viral envelope to enable viral entry into the host cells. Of note, it has been shown in mammalian cell expression systems that E1 and E2 form noncovalent heterodimers, whereas in the cell culture HCV system, the virion-associated E1-E2 complex can be linked covalently by disulfide bonds (Vieyres G, et al. Characterization of the envelope glycoproteins associated with infectious hepatitis C virus. J Virol (2010) 84(19):10159-68. doi: 10.1128/JVI.01180-10).

At the 5′ and 3′ ends of the HCV RNA, there are untranslated regions (UTRs) that are not translated into proteins but are important to translation and replication of the viral RNA. The 5′ UTR has a ribosome binding site or internal ribosome entry site (IRES) that initiates the translation of a very long protein containing about 3,000 amino acids. The core domain of the HCV IRES contains a four-way helical junction that is integrated within a predicted pseudoknot. The conformation of this core domain constrains the open reading frame's orientation for positioning on the 40S ribosomal subunit. The large pre-protein is later cleaved by cellular and viral proteases into the 10 smaller proteins that allow viral replication within the host cell or assemble into the mature viral particles. Structural proteins made by the hepatitis C virus include Core protein, E1 and E2; nonstructural proteins include NS2, NS3, NS4A, NS4B, NS5A, and NS5B.

The proteins of this virus are arranged along the genome in the following order: N terminal-core-envelope (E1)-E2-p7-nonstructural protein 2 (NS2)-NS3-NS4A-NS4B-NS5A-NS5B-C terminal. The mature nonstructural proteins (NS2 to NS5B) generation relies on the activity of viral proteinases. The NS2/NS3 junction is cleaved by a metal dependent autocatalytic proteinase encoded within NS2 and the N-terminus of NS3. The remaining cleavages downstream from this site are catalysed by a serine proteinase also contained within the N-terminal region of NS3.

The core protein has 191 amino acids and can be divided into three domains on the basis of hydrophobicity: domain 1 (residues 1-117) contains mainly basic residues with two short hydrophobic regions; domain 2 (residues 118-174) is less basic and more hydrophobic and its C-terminus is at the end of p21; domain 3 (residues 175-191) is highly hydrophobic and acts as a signal sequence for E1 envelope protein.

Both envelope proteins (E1 and E2) are highly glycosylated and important in cell entry. E1 serves as the fusogenic subunit and E2 acts as the receptor binding protein. E1 has 4-5 N-linked glycans and E2 has 11 N-glycosylation sites.

The p7 protein is dispensable for viral genome replication but plays a critical role in virus morphogenesis. This protein is a 63 amino acid membrane spanning protein which locates itself in the endoplasmic reticulum. Cleavage of p7 is mediated by the endoplasmic reticulum's signal peptidases. Two transmembrane domains of p7 are connected by a cytoplasmic loop and are oriented towards the endoplasmic reticulum's lumen. NS2 protein is a 21-23 kilodalton (kDa) transmembrane protein with protease activity. NS3 is 67 kDa protein whose N-terminal has serine protease activity and whose C-terminal has NTPase/helicase activity. It is located within the endoplasmic reticulum and forms a heterodimeric complex with NS4A-a 54 amino acid membrane protein that acts as a cofactor of the proteinase.

NS4B is a small (27 kDa) hydrophobic integral membrane protein with 4 transmembrane domains. It is located within the endoplasmic reticulum and plays an important role for recruitment of other viral proteins. It induces morphological changes to the endoplasmic reticulum forming a structure termed the membranous web.

NS5A is a hydrophilic phosphoprotein which plays an important role in viral replication, modulation of cell signaling pathways and the interferon response. It is known to bind to endoplasmic reticulum anchored human VAP proteins.

The NS5B protein (65 kDa) is the viral RNA dependent RNA polymerase. NS5B has the key function of replicating the HCV's viral RNA by using the viral positive RNA strand as its template and catalyzes the polymerization of ribonucleoside triphosphates (rNTP) during RNA replication. Several crystal structures of NS5B polymerase in several crystalline forms have been determined based on the same consensus sequence BK (HCV-BK, genotype 1). The structure can be represented by a right-hand shape with fingers, palm, and thumb. The encircled active site, unique to NS5B, is contained within the palm structure of the protein. Recent studies on NS5B protein genotype 1b strain J4's (HC-J4) structure indicate a presence of an active site where possible control of nucleotide binding occurs and initiation of de-novo RNA synthesis. De-novo adds necessary primers for initiation of RNA replication.

Hepatitis C virus entry is a complex and multistep process that involves interactions of the viral particles with cell surface glycosaminoglycans and many host factors, with the tetraspanin CD81, scavenger receptor class B member 1 (SR-B1), claudin-1, and occludin considered to be the essential set of entry factors. E2 may serve as the receptor binding protein of HCV and directly interacts with the CD81 and the SR-B1. In contrast, the role of E1 is poorly understood and appears to help modulate the E2-receptor interactions and fusion with the host cell membrane (Tzarum N. et al. Front. Immunol., 11 Jun. 2018, Sec. Vaccines and Molecular Therapeutics Vol. 9 (2018) doi: 10.3389/fimmu.2018.01315).

E2 is the main HCV receptor binding protein and the primary target for broadly neutralizing antibodies (bnAbs). The receptor binding domain (residues 384 to 645), also known as E2 core domain, is modified by up to 11 N-linked glycans (G. Gerold, et al., Hepatitis C virus entry: Protein interactions and fusion determinants governing productive hepatocyte invasion. Cold Spring Harb. Perspect. Med. 10, a036830 (2020) and contains several variable regions (VRs; comprising ~25% of the E2 core sequence) that increase the genetic diversity of HCV for evasion of the immune system (J. Prentoe, R. et al. Hypervariable region 1 and N-linked glycans of hepatitis C regulate virion neutralization by modulating envelope conformations. Proc. Natl. Acad. Sci. U.S.A. 116, 10039-10047 (2019); J. Prentoe, R. et al. Hypervariable region 1 shielding of hepatitis C virus is a main contributor to genotypic differences in neutralization sensitivity. Hepatology 64, 1881-1892 (2016)). Crystal structures of E2 from different HCV strains are all very similar, with the E2 core domain consisting of a central immunoglobulin (Ig) β-sandwich fold stabilized by eight disulfides and flanked by N-terminal hypervariable region 1 (HVR1; residues 384 to 410), a front layer (residues 421 to 459), and a C-terminal back layer (residues 597 to 645). The high density of the disulfide bonds contributes to the high thermal stability of E2, although the receptor binding site in recombinant E2 exhibits high conformational flexibility.

A detailed understanding of E2 and bnAb interactions at the molecular level is imperative for rational design of HCV vaccine antigens. Prior to the disclosure herein, most E2 bnAbs neutralize HCV by blocking E2-CD81 interactions and target three overlapping neutralizing sites (antigenic site (AS) 412 (amino acids 412 to 423), AS434 (amino acids 434 to 446), and antigenic region 3 (AR3), which collectively form the E2 neutralizing face. AR3 is a cluster of discontinuous epitopes formed by the front layer and CD81 binding loop (amino acids 519 to 535) and is the target for bnAbs isolated from infected patients. In the published E2 structures in complex with different AR3-specific bnAbs, E2 adopts a nearly identical conformation at the binding interface with the bnAbs. Given that the E2 neutralizing face (and CD81 binding site) is likely to be structurally flexible, the reported E2 structures appear to be in a “preferred” conformation recognized by bnAbs and thus a good template for structure-based design of vaccine immunogens (Netanel Tzarum et al. An alternate conformation of HCV E2 neutralizing face as an additional vaccine target. Sci. Adv. 6, eabbs5642(2020). DOI: 10.1126/sciadv.abb5642).

### Broadly Neutralizing Antibodies

Accordingly, the disclosure provides isolated broadly neutralizing HCV antibodies directed to a mixture of antigenically diverse E2 peptides. The neutralizing activity can be determined in an assay for determining the capacity to neutralize HCV pseudotype particles. Neutralizing assays are described in detail in the Examples section which follows.

Other methods for determining neutralizing ability include measuring the activity of a reporter gene product (e.g., luciferase, GFP). In certain embodiments, the antibodies neutralize HCV by at least 50%, e.g., at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% at an antibody concentration of about 500 μg/mL, about 400 μg/mL, about 300 μg/mL, about 200 μg/mL, about 100 μg/mL, about 90 μg/mL, about 80 μg/mL, about 75 μg/mL, about 70 μg/mL, about 60 μg/mL, about 50 μg/mL, about 40 μg/mL, about 30 μg/mL, about 25 μg/mL, about 20 μg/mL, about 15 μg/mL, about 10 μg/mL, about 5 μg/mL, about 2 μg/mL or about an 1 μg/mL.

In another embodiment, the neutralizing function is determined in an HCV cell culture system. In certain embodiments, the antibodies neutralize HCV by at least 50%, e.g., at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% at an antibody concentration of about 500 μg/mL, about 400 μg/mL, about 300 μg/mL, about 200 μg/mL, about 100 μg/mL, about 90 μg/mL, about 80 μg/mL, about 75 μg/mL, about 70 μg/mL, about 60 μg/mL, about 50 μg/mL, about 40 μg/mL, about 30 μg/mL, about 25 μg/mL, about 20 μg/mL, about 15 μg/mL, about 10 μg/mL, about 5 μg/mL, about 2 μg/mL or about an 1 μg/mL.

In certain embodiments, a bNAb comprises (i) a variable heavy chain having at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 1-57 or 58; and either (ii) a variable kappa light (Vκ) chain having at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 59-107 or 108; or (iii) a variable lambda light (Vλ) chain having at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 109-122 or 123, or combinations thereof. In certain embodiments, (i) the variable heavy chain (V_{H}) comprises SEQ ID NOs: 1-57 or 58; (ii) the variable kappa light (Vκ) chain comprises SEQ ID NOs: 59-107 or 108; or (iii) the variable lambda light (Vλ) chain comprises SEQ ID NOs: 109-122 or 123, or combinations thereof.

In another embodiment, an isolated variable heavy chain (V_{H}) comprises at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 1-57 or 58.

In another embodiment, an isolated variable kappa light (Vκ) chain comprises at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 59-107 or 108.

In another embodiment, an isolated variable lambda light (Vλ) chain comprises at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 109-122 or 123.

In another aspect, an isolated nucleic acid comprises: (i) a variable heavy chain encoded by a nucleic acid sequence having at least a 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 1-57 or 58; or (ii) a variable kappa light (Vκ) chain encoded by a nucleic acid sequence having at least a 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 59-107 or 108; or (iii) a variable lambda light (Vλ) chain encoded by a nucleic acid sequence having at least a 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 109-122 or 123, or combinations thereof. In certain embodiments, the isolated nucleic acid comprises (i) the variable heavy chain (V_{H}) encoded by a nucleic acid sequence comprising SEQ ID NOs: 1-57 or 58; or (ii) the variable kappa light (Vκ) chain encoded by a nucleic acid sequence comprising SEQ ID NOs: 59-107 or 108; or (iii) the variable lambda light (Vλ) chain encoded by a nucleic acid sequence comprising SEQ ID NOs: 109-122 or 123, or combinations thereof. In certain embodiments, the nucleic acid sequence encodes an amino acid sequence comprising one or more amino acid mutations comprising Y29F, I30S/T, R100aS, R100bG/E, Y100fA or combinations thereof.

Variablc Hcavy Chain Scqucnccs
hcab1_H (SEQ ID NO: 1):
caggtgcagctggtgcagtcaggggctgaggtgaagaagcctggggcctcagtgaaggtttcctgcaagaattctggatacaccctcagta
actctggcatgcattgggtgcgccaggcccccggacaaaggcctgagtggatgggatggataaacgttgccgaaggttacacaaaatattc
agagaagttgcaggccagactcaccataaccagtgacacagccgcgaacacagtctacatggagctgagcagcctgagatctgaagact
cggctgtgtattactgtgcgagtgttacgacgagacaatggttcgggaggggtgatgcttttgatctctggggccaagggacaatggtcacc
gcctcctca
hcab3_H (SEQ ID NO: 2):
caggtgcagctggtgcagtctggggctgaggtgaagaagcctgggtcctcggtgaaggtgtcctgcaaggcctctggaggcaccttcatg
aacaatgatatcaactgggtgcgacaggcccctggacaagggcttgagtggatgggagggagcattgctatgtctgggacaacaaactac
gcacagaagttccagggcagactgtcgataaccgcggacgattccacgggcacagcctacatggagctgaacagcctgagatctgacga
cacggccgtatattactgtgcgagagatctaatcgggttttgtagaggtggaaggtgctactcctggttcgacccctggggccagggaaccc
tggtcaccgtctcctca
hcab04_H (SEQ ID NO: 3):
caggtccagctggtgcaatctggggatgaggtgaagaagcctgggtcctcggtcaaggtctcctgcaagacttccggaggcaccttcgcc
atctataccatttcctgggtgcgacaggcccctggagtaggtcttgactggatggggagcatcacccctatggttggcagaacaaaatacgc
acagaacttccagggcagagtcacgattaccgcggacacatccacgaacacagtctacatggagctgagcagtctgagctctcaagacac
ggccatctatttctgtgcgtatcatggatcacaaatgttgggaacctatgacaatgccaactggttcgacccctggggccagggaaccccgg
tcaccgtctcctcag
hcab5_H (SEQ ID NO: 4):
caggtgcagctgcaggagtccggctcaggactggtgaggccttcacagaccctgtccctcacctgcactgtctctggtggctccatcagca
gtgatgcttactcctggaactggatccggcagccaccagggaagggcctggaatggattggtcacatctatcatagtggccgcacctccca
caacccgtccctcaacagtcgcgtcaccatctctgtagacaggtccaagaaccagttctccctgcggctgacctctgtgaccgccgcggac
acggccgtctattattgtgccagagaggcccaggactaccgtggctaccgtttcgactcctggggccagggaaccctggtcaccgtctcctc
ag
hcab6_H (SEQ ID NO: 5):
caggtgcagctggtgcagtctggacctgaggtgaagaagcctggggcctcagtgaaggtctcctgcaaggcctctggttacatctttacca
gccatggcatcacctgggtgcgccaggcccctggacaagggcttgagtgcatggggggatcagtgcttacagtggtcattcaaactctgc
acagaacttccagggcagagtcaccatgaccagagacacatccacgggcacagcctacatggaactgaggagcctgagatctgacgaca
cggccgtctactactgtgcgcgagcgtcagcggaatatggtttggacgtctggggccaagggaccacggtcaccgtctcctca
hcab7_H (SEQ ID NO: 6):
caggtgcagctggtgcagtctggagctgaggtgaagaagcctggggcctcagtgaaggtctcctgcaaggcttctggctacacctttacca
cctatggtatcacctgggtgcgacaggcccctggacaagggcttgagtggggggatggatcagcgcttacagtggtaacacaaactatgc
acaggaggtccagggcagagtcaccctgaccacagacacatccacgaccacatcctacatggaactgaggagcctgagatctgacgaca
cggccgtgtattactgtgcgagagtgaatgactacggtgcctacgtctttgacctctggggccagggaaccctggtcaccgtctcctca
hcab8_H (SEQ ID NO: 7):
caggtgcagctggtggagtctgggggaggcttggtccagccgggggggtccctgagactctcctgtgcagcctctggattcacctttaata
cctattggatgagctgggtccgccaggctccagggaaagggctggagtgggtggccaacataaagcaagatggaagcgagaaatactat
gtggactctgtgaagggccgattcaccatctccagagacaacgccaagaactcgctgtatctgcaaatgaacagcctgagagccgaggac
acggctgtgtattactgtgcgagggatagacccgcgggaattactatggttcggggaacgactattatagaggaggactactactacggtat
ggacgtctggggccaagggaccacggtcaccgtctcctca
hcab9_H (SEQ ID NO: 8):
caggtgcagctggtgcagtctggacctgaggtgaagaagcctggggcctcagtgaaggtctcctgcaaggcctctggttacatctttacca
gccatggcatcacctgggtgcgccaggcccctggacaagggcttgagtgcatggggtggatcagtgcttacagtggtcattcaaactctgc
acagaacttccagggcagagtcaccatgaccagagacacatccacgggcacagcctacatggaactgaggagcctgagatctgacgaca
cggccgtctactactgtgcgcgagcgtcagcggaatatggtttggacgtctggggccaagggaccacggtcaccgtctcctca
hcab10_H (SEQ ID NO: 9):
caggtgcagctggtggagtctgggggaggcttggtacagcctggggggtccctgagactctcctgtgcagcctctggactcagcttcagta
gtttctccattgactgggtccgccaaggtagaggagatagtctggagggggtctcactcactggtattgttgatgacacagttcccaggctcc
gtgaagggccaattcaccatctccagagaaaacggcacgacctccttgttgtttcatatgaacagcctgagagccggggacacaactgtgta
tcactgtacaggagatcggttgcattagtgtgacagtcgtgaaaagtagtcgctcgtcttggctcctggggccagggaaccctggtcaccgt
ctcctca
hacb11_H (SEQ ID NO: 10):
caggtgcagctggtggagtcggggggaggcttggtacagcctggggggtccctcacagtctcatgtgcagcctctggattctatttcagtgg
ctatagtatgaactgggtccgccaggctccagggaaggggctggagtgggtttcatacattagtagtagtggtagaaccattcactacgcag
actctgtgaggggccgattcaccatctccagagacaatgccaagaattcactgtatctgcgaatggacagcctgagagtcgaggacacgtc
tatctattactgtgcgagagatcaggggttgcgacattttgacacctacgactactactacgttctggacgtctggggccaagggaccacggt
caccgtctcctca
hcab12_H (SEQ ID NO: 11):
caggtgcagctggtgcagtctggggctgaggtgaagaagcctggggcctcagtgaaggtttcctgcaaggctgctggatacaccttcggta
cctatggggtgcattgggtgcgccaggcccccggacaaacgcttgagtggatgggatggatcaacgctggcaatgataacacaaaatattc
acagaagttccagggcagactcaccattaccagggacacatccgcgagcacagcctacatggacttgagcagcctgagatctgaagaca
cggctgtctattactgtgcgagagaaaaggacatgtactatggttcgccatgggacgtctggggccaagggaccacggtcaccgtctcctc
a
hcab13_H (SEQ ID NO: 12):
caggtgcagctgcaggagtcggggccaggactgataaagtcttcacagaccctgtccctcacctgcactgtctctggtggctccatcagtag
tggtggttattactggagctgggtccgccagcacccagggaagggcctggagtggattggaaacatcgatcacagcgggagaaccaact
acaacccgtccctccagagtcgcattagcgtttcaacagacacgtctaagaatgagttctccctgaagttgacctctgtgactgccgcggaca
cggccgtgtatttctgtgccagagggggccggggttacgatcttttgattggttcttatagagaggactactactatggtctggatgtctggg
gccaagggaccacggtcaccgtctcctca
hcab14_H (SEQ ID NO: 13):
caggtgcagctggtgcagtctggggctgaggtgaggaagcctggggcctcggtgaagatttcctgcaaggcatctggatactccttcacca
gctcctatatacactgggtgcgacaggcccctggacaagggcttgagtggatgggaataatcaaccctaagggtactggcgcaaagtatgc
acagaagttccagggcagagtcaccatgaccagcgacacgtccacgaatacagtccacatggagctgagcagcctgagatctggggaca
cggccgtctattactgtgcgagagcattaccaaattttgactacttatctgccctttattattggggccagggaaccctggtcaccgtctcct
ca
hcab15_H (SEQ ID NO: 14):
caggtgcagctggtgcagtctggggctgaggtgaagaagcctgggtcctcggtgaaggtctcctgcaaggcctctggaggcaccctcag
gctttctgaaataagttgggtgcgacaggcccccggacaagggcttgagtggatggcatatatcaatcctgtccttgggacagctatctacgc
acagaagttccagggcagggtcacgatttctgcggacgattctacgactacagcctacctggagctgaacagcctgagatctgaagacacg
gccgtctattactgtgcgagagaggagattaaatattgtcgtggcagtagttgctatgggtggttcgacccctggggccagggaactctggtc
accgtctcctca
hcab16_H (SEQ ID NO: 15):
caggtgcagctggtgcagtctggggctgaggtgaagaagcctggggcctcagtgaaggtttcctgcaaggcttctggatacatcttcactaa
ctatgctttgcattgggtgcgccaggcccccggacaaaggcttgagtggatgggatggatcaacgctgacagtagtaacacaatatattcac
agaagttccagggcagagtcaccattaccagggacacatccgcgagcacagcctacatggagctgagcagcctgagatctgaagacacg
gctgtgtattactgtgcgagagataaggtcatgtactatggttcgccatgggacgtctggggccaagggaccacggtcaccgtctcctca
hcab17_H (SEQ ID NO: 16):
caggtgcagctaaaccagtggggcgcaggactgttgaagccttcggagaccctgtccctcacgtgcgctgtctatggtgacgccttcagcg
gtaactactggagctggatccgccagcccccagggaaggggctgcagtggattggcgaaattaatcatagtggaaatactaattacgaccc
gtccctcgagagtcgagtcagtatatccttagacacgtccaagaaccagttgtccctgaaactgaactctgtgaccgccgcggacacggctc
tatattattgtgcgcgaggccagaggaattgtagtggtggtaactgctactcaggcctctgggcgtggggccagggaaccctggtcaccgtc
tcctca
hcab18_H (SEQ ID NO: 17):
caggtgcagctggtgcagtctgggccagaggtgaagaagcctgggtcctcggtaaaagtctcctgcaaggcttctggggacagcatcagc
aactatggtatcagctgggtgcgacaggcccctggacaagggcttgagtggatggctgcgatcatccctgtctttggtaaaacaaaatatgc
acagaagttccagggcagagtcacgattaccgcggacgaatccacgaccgcaggctacatggagctgaccaacctgagatttgaggaca
cggccgtctattactgtgcgatgaagctcctgccaccgcgactactccattacgggatggacgtctggggccaagggacctcggtcaccgt
ctcctca
hcab19_H (SEQ ID NO: 18):
caggtgcagctgcagcagtggggcgcaggactgttgaagccttcggagaccctgtccctcacctgctctgtctatggaggatccgtcactg
gttactactggagctggatccgccagcccccagggaaggggctggagtggattggcgaagttcatcatagtggaagcaccaagtacaacc
cgtccctcaagagtcgagtcaccatctcagtagacacgtccaagagccagttctccctgaagctgagctctgtgaccgccgcggacacgg
ctgtttattactgtgcgagagaggttgggttttacgatcttttgactgattccgcccgggtgggctactttgactactggggccagggatccc
tggtcaccgtctcctca
hcab20_H (SEQ ID NO: 19):
caggtgcagctggtggagtctgggggaggcttggtacagcctggggggtccctgagactctcctgtgcagcctctggattcacctttagca
gctatgccatgagctgggtccgccaggctccagggaaggggctggagtgggtctcaattattagcggtagtggtgatatcacatacaacgc
agactccgtgaagggccggttctccatctccagagacaattccgaagacactctatatcttcaaatgaacagtttgagagccgaggacacgg
ccgtatattactgtgcgaaatccccgggtatgattaggtttgcgggcgtcatcgttaaacctgtgtttgactcctggggccagggaaccctgg
tcaccgtctcctca
hcab21_H (SEQ ID NO: 20):
caggtgcagctggtgcagtctggagctgaggcgaagaagcctggggcctcagtgaaagtctcctgcaaggctgctggttacagctttatca
cctatggtatcaactgggtgcgacaggcccctggacaagggcttgagtggatgggatggatcagcgtttacagtggtaatacaaagtttgca
cagaaattccagggcagagtcaccatgaccagagatacatccacgagcacagcctacatggagctgaggagcctgagatctgacgacac
ggccgtgtattactgtgcgagagcgggggaagtctacggtgactattactttgactactggggccggggaaccctggtcaccgtctcctca
hcab22_H (SEQ ID NO: 21):
caggtgcagctggtgcagtctggggctgaggtgaagaagcctggggcctcagtgaaggtttcctgcaaggcttctggatacaccttcacttc
ctatgctatccattgggtgcgccaggcccccggacaaaggcttgagtggatgggatgcatcaacgctggcaatgataacacaaaatcttca
cagaagttccagggcagactcacctttagcagggacacatccgcgagtattgcctacatggagctgagcagcctgagatctgaagacacg
gctgtgtattactgtgcgaaggagcagtcgaattactatggttcgggggttgactactggggccagggaaccctggtcaccgtctcctca
hcab23_H (SEQ ID NO: 22):
caggtgcagctggtgcagtctggggctgaggtgaagaagcctgggtcctcggtgaaggtctcctgcaaggcttctggagacaccttcagc
ggctattctattacctgggtgcgacaggcccctggacaagggcttgagtggatgggagggatcatccctctctttgacacagcaaactacgc
acagaagttccagggcagagtcacgatgacggcggacgaatccacgaacacagcctacatggagctgagcagcctgagatctgaggac
acggccgtctattactgtgcgagagatccttggatagcagtgggcggagtggggtactttgaaaactggggccagggaaccctggtcacc
gtctcctca
hcab24_H (SEQ ID NO: 23):
caggtgcagctggtggaatctgggggagacttggtccagcctggggggtccctgagactcgcctgtgcagcctctggattcacctttagca
actatgccatgaactgggtccgccaggctccagggaaggggctagagtggctcgcaggtattaatgacagtggcgatttcacatactattca
gaccccgtgaaggcccgattcagcatctccagagacaattcgaagaacacgctttttctgcaaatgaacagcctgagagccgaggacacg
gccatatattactgtgcgaaagttgcccattcctattatcgaaaatcgcactttgacttctggggacagggaaccctggtcaccgtctcctca
hcab26_H (SEQ ID NO: 24):
caggtgcagctggtggagtctggggggggcgtggtccagcctgggaggtccctgagactctcatgtgtagcctctggattcagcttcgata
cctattctatgcactgggtccgccaggctccaggcaaggggctggagtgggtggcagttacatcatatgatggcaacgataaatactacgc
agactccgtgaagggccgattcaccatctccagagacaatttcaagaacatgctaaatctgcaattgaacagcctgagaactgaggacacg
gctgtgtatttctgtgcgagagatttcatagtggggtctaccttccgaaagcgggcgctctttgactactggggccagggaacccaggtcacc
gtctcctca
hcab27_H (SEQ ID NO: 25):
caggtgcagctggtgcagtctggagctgaggtgaagaagcctggggcctcagtgaaggtctcctgtaaggctcttggttacacctttaccag
ttatggtctcagctgggtgcgacaggcccctggacaggggcttgagtggatgggatggatcagcgcatacaatggtaacacaaagtacgc
acagaagttccagggcagagtcaccatgacctcagagacatccacgagtactgcctacatggaactgaggcgcctgagatctgacgacac
ggccgtgtattactgtgcgagagaaggggcagggactgtcgtgctgagagccaatgtgggacactcgtcccactacggaatggacgtctg
gggccaaggcaccacggtcaccgtctcctca
hcab29_H (SEQ ID NO: 26):
caggtgcagctgcagcagtggggcgcaggactgttgaagccttcggagaccctgtccctcacctgcgctgtccatggtcggtccttcattg
gttactactggagctggatccgccagcccccagggaaggggctggagtggattggggaagtcaatcatagtgggagctccaattacaacc
cgtccctcaagagtcgagtcaccatatcagcagagacgtccgaaaaccagttctccctgaagctgacctctgtgaccgccgcggacacgg
ctgtctattactgtgcgagagcgatagggtattacgatattttgactgattcttataacaccttccacttgactccctggggccagggaacc
ctggtcaccgtctcctca
hcab30_H (SEQ ID NO: 27):
caggtgcagctggtgcagtctggggctgaggtgaagaagcctgggtcctcggtgaaaatctcctgcaaggcttctggagtcagcttcagca
actttgctgtcagttgggtgcgacaggcccctggacaagggctggagtggatgggagggatcatccctttcttgggaaccacaaagtacgc
acagaagatccagggcagagtcacgattaccgcggacgaatccacgaccacagcctacatggagctgagcagcctgagatctgaggac
acggccgtatattactgtgcgagcgggttttgtagtggtggtagttgttacgtgaatttcatttacggtatggacgtctggggccaagggac
cacggtcaccgtctcctca
hcab31_H (SEQ ID NO: 28):
caggtgcagctggtggagtctgggggaggtgtggtacggccgggggggtccctgagactctcctgtgcagcctctggattcagctatgat
gactatgacatgaactgggtccgccaagctccagggaaggggctggagtgggtctctggtattacttggactggaagtaatagtggttacgc
agactctgtgaagggccgattcatcatctccagagacaacgccaagaagaccctatatctacaaatgaacagtctgggagtcgaagacacg
gccttgtactactgtgtgagggggtcgtattgtactagtaccagttgcgcgctctacgactatagaggtttggacgtctggggccaagggacc
gcggtcaccgtctcctca
hcab35_H (SEQ ID NO: 29):
caggtgcagctggtggagtcggggggaggcttggtacagccgggggggtccctgagactctcctgtgcagcctctggattcagctttagc
aactatgccatgagctgggtccgccaggctccagggaaggggctggagtgggtctcaggtttccgtggtagtgatggtagcacatactacg
cagactccgtgaagggccggttcaccatctccagagacaattccaagaacacactgtttctgcaaatgcacagcctgagagccgacgaca
cggccgtatattactgtgcgaaagatcagatcggccatacacctccccaacccggactagggatcggggagagcgaggactacttctacta
cgctatggacgtctggggccaagggaccacggtcaccgtctcctca
hcab36_H (SEQ ID NO: 30):
caggtgcagctggtggagtcggggggaggcgtggtccagcctgggcggtccctgagactctcctgtgaagtctctggaatccccttcagta
gttatggcatgcactgggtccgccaggctccaggcaaggggctggagtgggtgggtgttgtgtcatatgatggcacgaaaacatcctatgg
agactccgtgaagggccgattcaccatctccagagacaattccaagaacacgctgtatctgcaaatgaacagcctgaaaaatgaggacac
ggctgtgtatttttgtgcgaaagataaagcactcgtggtcgctcctaccccctacttctacttctacggtaaagggaccacggtcaccgtct
cctca
hcab37_H (SEQ ID NO: 31):
caggtgcagctggtgcagtctggacctgaggtgaagaggcctggggcctcagtgaaggtctcctgcaagacctccggttacatctttacta
gtcatggtatcagctgggtgcgacaggcccctggacaagggcttgagtggatgggatggatcagcccttacagtggtaacacaaagactgt
acagaagctccagggcagagtcaccctgaccgcagacacatccacgaaaacagcctacatggagctgaggagcctgacatcagacgac
acggccgtctattattgtgcaagagcggatgggaactactacgtcatggacgtctggggccaagggaccacggtcaccgtctcctca
hcab39_H (SEQ ID NO: 32):
caggtgcagctgcaggagtcggacccaggactggtgaaaccttcacagaccctgtccctcacctgcactgtctctggtggctccatcaaca
gtaatgattactactggagttggatccgccagcccccagggaagggcctggagtggattgggtacatctcttacagtgggaccacctactac
aacccgtccctcaagagtcgaatttccatatcagttgacacgtccaagaaccagttctccctgaatctgagctctgtgactgccgcagacacg
gccgtatattactgtgccagagagagggggggctcctggttatccgtggatgcagctattattactggggcgtttgactactgtcaccgtct
cctca
hcab40_H (SEQ ID NO: 33):
caggtgcagctacagcagtggggcgcaggactgttgaagccttcggagagcctgtccctcacctgcgtagtctatggtgggtccttcagtg
gttactactggagctggatccgccagcccccagggaaggggctggagtggattggggaaatcaatcctactggaagcaccaactacaatc
cgtccctcaacagtcgagtcaccatatcagtagacaggtccaagaaccacttgtccctgaagctgagctctgtcaccgccgcggacacggc
tgtgtatttctgtgcgagatttctcccccccgactacgatcttttgactgattattttgaccccccgggtgcttttgatatctggggccaag
ggacaatggtcaccgtctcctca
hcab41_H (SEQ ID NO: 34):
caggtgcagctggtgcagtctggggctgaggtgaagaagcctgggtcctcggtgaaggtctcctgcaaggcttctggaggctccttcagca
atgatcctattagttggatccgacaggcccctggacaagggcttgagtggatgggagggatcatccctaaacttggtaaatcaacttacgctc
agaagttccagggcagagtcacgattaccgcggacgaagccgcgagcacaggttacatggagctgagcagcctgagatctgaggacac
ggccgtgtattactgtgcgagaggaggccgcgtattgcgacattttgactggctagtgtacttctggggcaaagggaccacggtcaccgtct
cctca
hcab42_H (SEQ ID NO: 35):
caggtgcagctacagcagtggggcgcaggactgttgaagccttcggagaccctgtccctcagctgcgctgtctatggtgggtccttcagtg
gttactactggagttggatccgccagtccccagggaaggggctggagtggattggggaaatcaatcatagtggaagcaccaactacaacc
cgtccctcaagagtcgagtcaccatatcagtagacacgtccaagaaccagttctccctgaagctgagctctgtgaccgccgcggacacggc
tgtctattactgtgcgagagatacgggatattacgatattttgactgattatttttcaagggggggcaaagggaccacggtcaccgtctcct
ca
hcab43_H (SEQ ID NO: 36):
caggtgcagctggtgcagtctggggctgaggtgaagaagcctgggtcctcggtgaaggtctcctgcaaagcttctggaggctccttcagta
actatggtatccactgggtgcgacaggcccctggactagggcttgagtggatgggagggatcatccctaccttacgtacaccaaactacgc
acagaaattccagggcagagccacgattaccgcggacgaatcctcgaacacatcctacatggagctgagcagcctgagatctgaagaca
cggccgtgtattactgtgcgagggatccgggtatagtatcaactggtatttactacaccggtatggacgtctggggccaagggaccacggtc
accgtctcctca
hcab44_H (SEQ ID NO: 37):
caggtgcagctggtgcagtctggagctgaggttcagaagcctggggcctcagtgaaggtctcctgcaaggcttctggttatttttttaccag
ccatggtatcgtctgggtgcgccaggcccctggacaagggcttgagtggatgggatggatcagcccttacagtggcaacacaaagtatgca
cagaaactccagggcagagtcaccatgaccacagacacatccacgagcacagcctacatggagctgaggagcctgagatctgacgaca
cggccgtgtatttctgtgcgagagcgggagaagtctacggtgactactacttcgactactggggccagggaaccctggtcaccgtctcctca
hcab45_H (SEQ ID NO: 38):
caggtgcagctggtacagtctggggcagaggtgaagaagcctgggacctcagtgaaggtttcctgcaaggcatctggatacaccttcacc
agtgtctatatgcactgggtgcgacagggccctggacaagggcttgagtggatgggaataatcaaccctagagatggtagcacaaactac
gcacagaaattccagggcagagtcaccatgaccagggacacgtccacgagcacagtctacatggagctgagcaccctgagatccgacg
acacggccgtgtattactgtgcgcgaacattaccatcttttgactggttatctgccctttattactggggccagggaaccccggtcaccgtc
tcctca
hcab47_H (SEQ ID NO: 39):
caggtgcagctggtgcagtctggggctgaggtgaagaagcctggggcctcagtgaaggtttcctgcaaggcatctggatacaccttcacca
gctactatatgtactgggtgcgacaggcccctggacaagggcttgagtggatgggaataatcaaccctagtggtgatagcacaagctacgc
acagaagttccagggcagagtcaccatgaccagggacacgtccacgagcacagtctacatggagctgagcagcctgagatctgaggaca
cggccatgtattactgtgtctactactactacggtatggacgtctggggcaaagggaccacggtcaccgtctcctca
hcab48_H (SEQ ID NO: 40):
caggtgcagctggtggagtctgggggaggcttagctcagccgggggggtccctgagactctcctgtgcagcctctggattcgccttcagta
gctcctggatacactgggtccgccaagctccggggaaggggctggtgtggctctcacgtgttaattatgatgggattagcacaacttacgcg
gactccgtgaagggccgattcaccatctccagagacaacgccaagagcacggtgtttctgcaaatgaacagtctgagaggcgaggacac
ggctgtgtattactgtgcaacatcgtacttatctcgtggccccatgggcgactggggccagggaaccctggtcaccgtctcctca
hcab49_H (SEQ ID NO: 41):
caggtgcagctggtgcagtctggggctgaggtgaagaagcctggggcctcagtgaaggtttcatgcaaggcatctggatacaccttcacca
gttactatatgcactgggtgcgacaggcccctggacaagggcttgagtggatgggaataatcaaccctagtggtggtagcacaacctacgc
acagaagttccagggcagactcaccatgagcagggacacgtccacgagcacagtcaacatggagctgagcagcctgagatctgaggac
acggccgtgtattactgtgcgagaggggtcgggcacgggtgggtcggggaggtattatatcgggatgctgataaccactacggtatggac
gtctggggccaagggaccacggtcaccgtctcctca
hcab50_H (SEQ ID NO: 42):
caggtgcagctggtgcagtctggggcagaggtgaagaagactgggtcttcggtgaaggtctcctgcaaggcttctggaggccccgtcaac
agctatggtataagttgggtgcgacaggcccctggacaagggcttgaatggatgggagcaatcatccctgtttttggtagagtaaaatacgc
acagaagttccagggcagagtcacgattaccgcggacgagtccacgaccacagccaacatggaagtgaccagcctgagatttgaggaca
cggccgtctattactgtgcgatgaagttagaagtcccgcgacttttgcattatggtatggacgtctggggccaaggaaccacggtcaccgtct
cctca
hcab51_H (SEQ ID NO: 43):
caggtgcagctacagcagtggggcgcaggactgttgaagccttcggagaccctgtccctcacctgcgctgtccatggtcggtccttcattgg
ttactactggagctggatccgccagcccccagggaaggggctggagtggattggggaagtcaatcatagtgggagctccaattacaaccc
gtccctcaagagtcgagtcaccatatcagcagagacgtccgaaaaccagttctccctgaagctgacctctgtgaccgccgcggacacggct
gtctattactgtgcgagagcgatagggtattacgatattttgactgattcttataacaccttccactttgactcctggggccagggaaccct
ggtcaccgtctcctca
hcab54_H (SEQ ID NO: 44):
caggtgcagctacagcagtggggcgcaggactattgaagccttcggagaccctgtccctcacctgcgctgtctatggtgggtccttcagtg
gttactactggaactggatccgccagtccccagggaaggggctggagtggattggggaaatcaatcatagtgggagcaccaactacaacc
cgtcactcaagagtcgagtcaccatgtcagtagacacgtccaagaaccagttctccctgaagctgagctctgtgaccgccgcggacacgg
ctgtgtattactgtgcgagaggcctggggtattacgatactgtgactggttatatcgggtcgggaggtatggacgtctggggccaagggacc
acggtcaccgtctcctca
hcab55_H (SEQ ID NO: 45):
caggtgcagctggtgcaatctggggctgaaattaagaagcctggggcctcagtgaaggtttcctgcaaggcatctggatacaccttcatcag
taactatttccactgggtgcgacaggcccctggacaagggcttgagtggatgggaataatcaaccctagttctggtagtacaacctatggac
agaaattccagggcagagtcaccctgactagcgacacgtccgcgagcatagtctacctggagctgagccgcctgagatctgaagacacg
gccgtgtattattgtgttagtccccggatagcacattgtcgtggtggtcggtgctacgagactctcgactggggccagggaaccctggtcac
cgtctcctca
hcab57_H (SEQ ID NO: 46):
caggtgcagctggtgcagtctggggcagaggtgaagaagcctgggtcctcggtgagggtctcctgcaaggcttctggaggcaccttcagc
agctatggtatcagctgggtgcgacaggcccctggacaagggcttgagtggctgggagggatcatccctacgtttggtagagcaaaatac
gcacagaagttccagggcagagtcacgattaccgcggacgaatctacgtccgcttccaacatggaggtgaccagtctgagatttgaggac
acggccgtgtattactgtgcgataaagattctaccaccacgactgctcttttacggaatggacgtctggggcccagggaccacggtcaccgt
ctcctca
hcab58_H (SEQ ID NO: 47):
caggtgcagctgcagcagtggggcgcaggactgttgaagccttcggagaccctgtccctcacctgcgctgtctatggtgcgcccttcagtg
gttactactggaactggatccgccagcccccagggaaggggctggagtggattggggaactcaatcatagtggaggcaccaactacaac
ccgtccctcaagagtcgaatcaccatgtctgtagacccgtccaacaaccagttttccctgaggttgagctctgtgaccgccgcggacacggc
tgtgtatttctgtgcgaggtccctgccggccgattacgatcttttgactggttctaatgatttattcggtgcttttgatctctggggccaag
ggaccgtggtcaccgtctcctca
hcab59_H (SEQ ID NO: 48):
caggtgcagctggtggagtcggggggaggcttggtacagccgggggggtccctgagactctcatgtgcaggctctggattctacttcagtg
cctatagcatgaactgggtccgccaggctccagggaaggggctggagtgggtttcatacattagtagtagtggtagaaccatttactacgca
gactctgtgaggggccgatttaccatctccagagacaatgccaagaattcactgtatctgcgaatggacagcctgagagtcgaggacacgg
ctatctattactgtgcgagagatcaggggttgcgacattttgacacctacgactactactacgttatggacgtctggggccaagggaccacg
gtcaccgtctcctca
hcab60_H (SEQ ID NO: 49):
caggtgcagctggtggagtctgggggaaatatggtacggcctggggggtccctgagactctcctgtgcagcctctggattctcgtttgatga
ctatgggatgagttgggtccgccacgttccagggaagggactggagtgggtcgctggtattaattggaagggtgatagtataaattatgcag
actctgtgaggggccgattcactatctccagagacaacgccaagaactccctgtatctgcaaatgaacagtctgagagccgaggatacggc
cttgtatcactgtgcgagaggagacattactttgggtcggggagtttattactcttatcacggtttggacgtctggggccaagggaccacgg
tcaccgtctcctca
hcab61_H (SEQ ID NO: 50):
caggtgcagctgcaggagtccggctcacgactggtaaacccttcagagaccctgtccctcacctgcgaagtctctggtggctccatcagta
gcggtgattactcctggacctggatccggcagccgccagggaaggacctggagtggattgggtacatctaccatagtgggagcacctattt
caacccgtccctcaagagtcgagtcagcatgtcagtagacagttccaagaaccagctctccctgcacctgaggtctgtgaccgccgcggac
acggccgtatattactgtgccagactcagtaacatgtggcccaagtacaaccacatcgactcctggggccagggaaccctggtcaccgtct
cctca
hcab64_H (SEQ ID NO: 51):
caggtgcagctggtgcagtctggggctgaaatacagaagcctggggcctcagtgaaggtttcctgcagggcatctggatacacttacatca
gcacctctttccactgggtgcgacaggcccctggacaggggcttgagtggatgggagtaatcaagactgatgatggtagtaccagcgaca
cacagaagttccagggcagactcaggatgacctgggacacgtccacgagcacagtttacatggaactgagcagcctgagatctgacgac
acggccgtgtattattgtgttagtccccgcctaggacattgtcgtcgtggccggtgctacgagactctcgactggggccagggatccctggtc
accgtctcctca
hcab65_H (SEQ ID NO: 52):
caggtgcagctggtggagtctgggggaggcttggtacagcctggggggtccctgagactctcctgtgaagcctctggattcaccttcagta
actataacatgaactgggtccgccaggctccagggaaggggctggagtgggtttcatatattagtagtagtagtagtaccatatattacacag
actctgtgaagggccgattcaccatctccagagacaatgccaagaactcactgtatctgcaaatgagcaggctgagagccgaggacacgg
ctgtgtattactgtgcgagagcggacgatcccacgacaattctagtccccggaaatatcgcagcacctggactcgaaacagagttagggttg
gatgaccaccagtaccgcggtatggacgtctggggccaagggaccacggtcaccgtctcctca
hcab66_H (SEQ ID NO: 53):
caggtgcagctggtgcagtctggacctgaggtgaagaagcctggggcctcagtgaaggtctcctgcaaggcttctggttacatctttaccag
tcatggtatcacctgggtgcgacaggcccctggccaagggcttgagtggatgggatggatcagcgcttacagtggtaacacaaactatcca
cagaaggtccaggacagagtcaccatgaccagagacacatccacgagcacagcctacatggagttgaggagcctgagacctgacgaca
cggccgtatattattgtgtgagaagtggaaactacccgttcatcttcgacccctggggccagggaaccctggtcaccgtctcctca
hcab67_H (SEQ ID NO: 54):
caggtgcagctggtggagtctgggggaggcttggtacagccgggggggtccctgagactctcctgtgcagcctctggattcgcctttagca
cctatgtcatgagctgggtccgccaggctccagggaagggtctggagcggatctcgtctattagtggtcgtggaacaacgtacgcagactc
cgtgaagggccgattcaccgtctccagagacaattccaagaacacgttgtatctgcaaatgaacagtctgagagccgaggacacggccgt
atattactgtgcaaaagagggcgccgagatacgacttttggactggccgatattgatagactggtacttcgatctctggggccgtggcaccct
ggtcaccgtctcctca
hcab68_H (SEQ ID NO: 55):
caggtgcagctggtgcagtctggagctgaggtggtgaagcctggggcctcagtgagagtctcctgcaaggcttctggttacatctttaccag
ccatgggatcgcctgggtgcgacaggcccctggacaagggcttgagtcgatgggatggatcagcgcttacagtggaaacacaaactatgc
acagaaattccagggcagagtcacaattaccagagacacatccacgagcacagcctatttggagttgaggagcctccgatctgacgacac
ggccgtatactactgtgcgagagtggagcaattgctactaggggacttctggggccagggaaccctggtcaccgtctcctca
hcab71_H (SEQ ID NO: 56):
caggtgcagctggtgcagtctggggcagaggtgaagaagactgggtcttcggtgaaggtctcctgcaaggcttctggaggccccgtcagc
agctatggtataagttgggtgcgacaggcccctggacaagggcttgaatggatgggagcaatcatccctgtttttggtagagtaaaatacgc
acagaagttccagggcagagtcacgattaccgcggacgagtccacgaccacagccaacatggaagtgaccagcctgagatttgaggaca
cggccgtctattactgtgcgatgaagttagaagtcccgcgacttttgcattatggtatggacgtctggggccaaggaaccgcggtcaccgtct
cctca
hcab73_H (SEQ ID NO: 57):
caggtgcagctggtgcagtctggggcagaggtgaagaagtctgggtcctcggtgaaggtctcctgcaaggcttctggagacaccttcagta
gttatggtatcacctgggtgcgccaggcccctggacaagggcttgagtggatgggaggtatcatccctgtgtttggaagagcaaaatacgc
acagaagttccagggccgagtcaccattaccgcggacgaatccacgaccacaggctacatggaggtgaccagactgagatttgaagaca
cggccgtatattattgtgcgataaaggtcgaagcaccgcgactactcttttacggtatggacgtctggggccaagggaccacggtcaccgtc
tcctca
hcab74_H (SEQ ID NO: 58):
caggtgcagctggtgcagtctggagcagaggtgaaaaagcccggggagtctctgaagatctcctgtaagggttctggatacagtttcacca
gttactgggtcggctgggtgcgccagatgcccggaaaaggcctggagtggatggggatcatccatcctgatgtgtctgaaattagatacag
cccgtccctccaaggccacgtcaccatgtcagtcgacaagtccatcagtaccgcctacctgcagtggagcagcctgaaggcctcggacac
cgccatttattactgtgcgagacatgggtatgggtcgggcttctttgacaactggggccagggaaccctggtcaccgtctcctca
Variablc Kappa Chains
hcab49_K (SEQ ID NO: 59):
gatgttgtgatgactcagtctccactctccctgcccgtcacccttggacagccggcctccatctcctgcaggtctggtcaaagcctcgtacac
agtgatggaaacacctacttgaattggtatcaacagaggccaggccaatctccaaggcgcctaatttataaggtatctaaccgggaccctgg
ggtcccagacagattcagcggcagtgggtcaggcactgatttcacactgaaaatcagcagggtggaggctgatgatattggagtttattact
gcatgcaaggtacatactggcctgagatcaccttcggccaagggacacgactggacattaaac
hcab74_K (SEQ ID NO: 60):
gatattgtgatgactcagtctccactctccctgcccgtcacccctggagagccggcctccatctcctgcaggtctagtcagagcctcctgcat
agtaatggatacaactatttggattggtatctgcagaagccagggcagtctccacagctcctgatctatttgggttctaatcgggcctccggg
gtccctgacaggttcagtggcagtggatcaggcacagattttacactgaaaatcagcagagtggaggctgaagatgttgggatttattactg
catgcagggtctacaaacctcgttcactttcggcggagggaccaaggtggagatcaaac
hcab60_K (SEQ ID NO: 61):
gatattgttatgacccagtctccattctccctgcccgtcacccctggagagccggcctccatctcctgcaggtcaagtcagagcctcctgca
tagtaatggatacaactatttggattggtacctgcagaagccagggcagtctccacagctcctgatctatttgggttctaatcgggcctccg
gggtccctgacaggttcagtggcagtggatcaggcacagattttacactgaaaatcagcagagtggaggctgcggatgttgggatttattact
gcatgcaggctctacaaactccgacgttcggccaagggaccaaggtggaaatcaaac
hcab31_K (SEQ ID NO: 62):
gatattgtgatgactcagtctccactctccctgcccgtcacccctggagagccggcctccatctcctgcaggtctagtcggagcctcctgca
ttttaatggatacgactatttggattggtacctgcagaagccagggcagtctccacagctcctgatctatttgggttctaatcgggcctctg
gggtccctgacaggttcagtggcagtggatcaggcacagattttacactgaaaatcagcagagtggaggctgaagatgttggggtttattac
tgcatgcaagctctacaaactcctcgcacgttcggccaagggaccaaggtggaaatcaaac
hcab8_K (SEQ ID NO: 63):
gatattgtgatgactcagtctccagtctccctgcccgtcacccctggagagccggcctccatctcctgcaggtctagtcagagcctcctgcat
agtaatggatacaactatttggattggtacctgcagaagccagggcagtctccacagctcctgatctatttgggttcttatcgggcctccgg
ggtccctgacaggttcagtggcagtggatcaggcacagattttacactgagaatcagcagagtggaggctgaggatgttggggtttattact
gcatgcaagctctacaaactcctcggacttttggccaggggaccaagctggagatcaaac
hcab13_K (SEQ ID NO: 64):
gatattgtgatgactcagtctccactctccctgcccgtcacccctggagagccggcctccatctcctgcaggtctagtcagagcctcctgcac
agtaatggatacaactatttggattggtacctgcagaagccagggcagtctccacagctcctgatctatttgggttcttatcgggccgccggg
gtccctgacagattcagtggcagtggatcaggcacagattttacactgaaaatcagcagagtggaggctgaggatgttggggtttattactgc
atgcaagctctacaaactccgtggacgttcggccaagggaccaaggtggaaatcaaac
hcab47_K (SEQ ID NO: 65):
gatattgtgatgactcagtctccactctccctgcccgtcacccctggagagccggcctccatctcctgcaggtctagccagagcctcctgcac
agtaatggatacaactatttggattggtacctgcagaagccagggcagtctccacagctcctgatctatttgggttcgtatcgggccgccggg
gtccctgacagattcagtggcagtggatcaggcactgattttacactgcaaatcagcagagtggaggctgaggatgttggcctttattactgc
atgcaagctctacaaactccgtggacgttcggccaagggaccaaggtggatatcaaac
hcab40_K (SEQ ID NO: 66):
gacatccatatgacccagtctccattctccctgtctgcatctgtaggagacagagtcaccatcacttgccgggccagtcagagtattagtgg
ctggttggcctggtatcagcagaaagtagggaacgcccctaagctcctgatctataaggcgtctagtttagaaagtggggtcccatcaagat
tcagcggcagtggatctgggacagaattcactctcagcatcagcagcctgcagcctgatgattttgcaacttattactgccaacagtataat
agttacccattcactttcggccctgggaccaaagtggatatgaaac
hcab41_K (SEQ ID NO: 67):
gacatccatatgacccagtctccattctccctgtctgcaactgttggagacagagtcaccatctcttgccgggccagtcagagtattggtgg
ctggttggcctggtatcagcagaaaccagggaaagcccctaagctcctgatctatcaggcgtctaccttagaaactggggtcccatcaaggt
tcagcggcagtggatctgggacagaattcactctcaccatcagcagcctgcagcctgatgattttgcaacttattactgccaacactataat
agttatttattcactttcggccctgggaccaaagtggatatcaaac
hcab11_K (SEQ ID NO: 68):
gacatccagatgacccagtctccattctccctgtctgcatctgtaggagacagagtcaccatcacttgccgggccagtcagagtattagtagc
tggttggcctggtatcagcagaaaccagggaaagcccctaagctcctgatctataaggcatctagtttacaaagtggggtcccatcaaggttc
agcggcagtggatctgggacagaattcactctcaccatcagcagcctgcagcctgatgattttgcaacttattactgccagcagtatcatact
gattggtacacttttggccaggggaccaaggtggagatcaaac
hcab57_K (SEQ ID NO: 69):
gacatccagatgacccagtctccttccaccctgtctgcatctgtaggagacagagtcaccatcacttgccgggccagtcagagtattagtaac
tggttggcctggtatcagcagaaaccagggaaagcccctaaactcctgatctataaggcatctagtttagaaagtggggtcccatcaaggttc
agcggcagtggatctgggacagaattcactctcaccatcaacagcctgcagcctgatgattttgcaacttatcactgccaacaatacaaaagt
tattggaacagttttggccaggggaccaagctggagatgaaac
hcab58_K (SEQ ID NO: 70):
gacatccagatgacccagtctccttccaccctgtctgcatctgtaggagacagagtcaccatcacttgccgggccagtcagagtattagtaac
tggttggcctggtatcagcagaaaccagggaaagcccctaaactcctgatctataaggcatctagtttagaaagtggggtcccatcaaggttc
agcggcagtggatctgggacagaattcactctcaccatcaacagcctgcagcctgatgattttgcaacttatcactgccaacaatacaaaagt
tattggaacagttttggccaggggaccaagctggagatgaaac
hcab59_K (SEQ ID NO: 71):
gacatccagatgacccagtctcctttctccctgtctgcatctgtaggagacagagtcaccatcacttgccgggccagtcagagtattagtaac
tggttggcctggtatcagcagaaaccagggaaagcccctaaactcctgatctataaggcatctagtttagaaagtggggtcccatcaaggttc
agcggcagtggatctgggacagaattcactctcaccatcaacagcctgcagcctgatgattttgcaacttatcactgccaacaatacaaaagt
tattggaacagttttggccaggggaccaagctggagatgaaac
hcab50_K (SEQ ID NO: 72):
gacatccatatgacccagtctccagtctccctgtctgcatctgtaggagacagagtcaccatctcttgccgggcgagtcaggacattagtact
tatcttgcctggtatcagcagagaccaggggaagttcctaagctcctgatctatgctgcatccactttgcaatcaggggtcccctctcggttc
agtggcagtggatctgggacagatttcactctcaccatcagcagcctgcagcctgaagatgttgcaacttattactgtcaaaagtataacag
tgcctcctcgatcaccttcggccaagggacacgactggatattaaac
hcab54_K (SEQ ID NO: 73):
gccatccagatgacccagtctccatcctccctgtctgcatctgtaggagacagagtcaccatcacttgccgggcaagtcagggcattagaaa
tgatttaggctggtatcagcagaaaccagggaaagcccctaaactcctgatctatgctgcatccaatttacaaagtggggtcccatcaaggtt
caacggcagtggatctggcactgatttcactctcaccatcaacagtctgcacctgaagattttgcaacttattactgtgtacaagactacaa
tttccctcgcacttttggccaggggaccaagctggagatcaaac
hcab15_K (SEQ ID NO: 74):
gccatccatatgacccagtctccatcctccctgtctgcatctgtaggagacagagtcaccatcacttgccgggcaagtcagggcattagaaat
gatttagcctggtatcagcataaaccagggacagcccctaaggtcctgatttatgctgcttccagtctacaagatggggtctcatcaaggtt
cagcggcagtggatctggcacattattcactctcaccatcagcagcctgcagcctgaagattttgcaacttattactgtctacaaggttaca
attatcctcggacttttggccaggggaccaaggtggagatgaaac
hcab17_K (SEQ ID NO: 75):
gccatccatatgacccagtctccattctccctgtctgcatctgtaggagacagagtcaccatcacttgccgggcaagtcagggcattagaaat
gatttagcctggtatcagcataaaccagggacagcccctaaggtcctgatttatgctgcttccagtctacaagatggggtctcatcaaggtt
cagcggcagtggatctggcacattattcactctcaccatcagcagcctgcagcctgaagattttgcaacttattactgtctacaaggttaca
attatcctcggacttttggccaggggaccaaggtggagatgaaac
hcab42_K (SEQ ID NO: 76):
gccatccagatgacccagtctccattctccctgtctgcatctgtaggagacagagtcaccatcacttgccgggcaagtcagggcattagaaat
gatttaggctggtatcagcagaaaccagggaaagcccctaagctcctgatctatgctgcatccagtttacaaagtggggtcccatcaaggttc
agcggcagtggatctggcacagatttcactctcaccatcagcggcctgcagcctgaagattttgcaacttattactgtctacaagtttacaa
ttatcctcggacgttcggccaagggaccaaggtggaaatcaaac
hcab43_K (SEQ ID NO: 77):
gccatccagatgacccagtctccatcctccctgtctgcatctgtaggagacagagtcaccatcacttgccgggcaagtcagggcattagaaa
tgatttaggctggtatcagcagaaaccagggaaagcccctaagctcctgatctatgctgcatccagtttacaaagtggggtcccatcaaggtt
cagcggcagtggatctggcacagatttcactctcaccatcagcggcctgcagcctgaagattttgcaacttattactgtctacaagtttaca
attatcctcggacgttcggccaagggaccaaggtggaaatcaaac
hcab51_K (SEQ ID NO: 78):
gccatccagatgacccagtctccatcctccctgtctgcatctgtaggagacagagtcaccatcacttgccgggcaagtcagggcattagaaa
tgatttaggctggtatcagcagaaaccagggaaagcccctaagctcctgatctatgctgcatccagtttacaaagtggggtcccatcaaggtt
cagcggcagtggatctggcacagatttcactctcaccatcagcggcctgcagcctgaagattttgcaacttattactgtctacaagtttaca
attatcctcggacgttcggccaagggaccaaggtggaaatcaaac
hcab19_K (SEQ ID NO: 79):
gacatccagatgacccagtctccattctccctgtctgcatctgtgggagacagagtcaccatcacttgccaggcgagtcaggacattaccaa
ctatttaagttggtatcagcagaaaccagggacagcccctaaactcctgatctacgatgcatccaatttggaaacaggggtcccatcaaggtt
cagtggaagtggaactgggacacattttactttcaccatcagcagcctgcagcctgaagatattgcaacatattactgtcaacagtatgata
ctctcccgctcactttcggcggagggaccaaggtggagatcaaac
hcab68_K (SEQ ID NO: 80):
gacatccatatgacccagtctccagtctccctgtctgcatctgtgggagacagagtcaccatcccttgccgggcaagccatcccattaacaac
tatttaaattggtatcagcagaaaccagggaaagcccctaacctcctgatctatgcttcatccaatttgcattctggggtcccatcgaggtt
cagtgccagtggatctggaacaaatttcactctcaccatcagcagtctacaacctgaagattttgcaatttacttctgtcaacagacttcca
gtacccctaacactttcggcggagggaccagggtcgacatcagac
hcab9_K (SEQ ID NO: 81):
gacatccagatgacccagtctccatcctccctgtctgcatctgttggcgacagagtcatcatcacttgccgggcaagtgaaagcatcagtacc
tacttaggttggtatcagcagaaaccagggaaagcccctaaactcatcatctctgctgtttccactttgcaaagtggggtcccctcaaggtt
cagtggcagtggatctggaacagaattcactctcaccatcagcagtctgcaacctgaagattttgcaacttactactgtctacagagtcaca
gtatccctcaaacattcggccctgggaccacagtggatatcaagc
hcab27_K (SEQ ID NO: 82):
gacatccagatgacccagtctccatcctccctgtctgcatctgtaggagacagagtcaccatcacttgccgggcaagtcagagcattggcag
caatttaaactggtatcaggcgaaaccagggaaagcccctaggctcctgatctattctgcatccactttgcaaagtggggtcccatcaacgtt
cagtggcagtggatctgggacagacttcactctcgccatcagcagtctgcaacctgaggattttgcaacttattattgtcaacagagttaca
gtgcccctagtttcggcggagggaccacggtggagatcaaac
hcab3_K (SEQ ID NO: 83):
gacatccagatgacccagtctccattctccctgtctgcatctgtaggagacagagtcaccatcacttgccgggcaagtcagagtattagtaac
tatttaaattggtatcagcagaaatcagggaaagcccctaaaatcctgatctctgctgcatccactttgcaaagtggggtcccatcaaggtt
cagtggcagtggatctgggacagatttcactctcaccatcagcagtctgcaacctgaagattttgcaacttactactgtcaacacagtgac
agtacccgtggtttcatttttggccgggggaccaagctggagatcaagc
hcab26_K (SEQ ID NO: 84):
gacatccagatgacccagtctccattctccctgtctgcatctgtaggagacagagtcaccatcacttgccgggcaagtcagaccattagcagt
tatttaaattggtatcagcagaaaccagggaaagcccctaaactcctgatgtttgctgcatccactttacaaagtggagtcccatcaaggtt
cagtggcagtggatctgggacagatttcactctcaccatcagcggtctactacctgaagattttgcaacttactactgtcaacagagttac
agtagcccgctcactttcggcggagggaccaaggtggagatcaaac
hcab5_K (SEQ ID NO: 85):
gacatccagatgacccagtctccatcctccctgtctgcatctgtaggagacagagtcaccatcacttgccgggcaagtcagtacattagtgac
tatttaaattggtatcagcagaaaccagggaaagcccctaacctcctgatctatgctgcatccagtttgcaaagtggggtcccatcaaggtt
cagtggcagtggatctggcacagatttcactctcaccatcagcagtctgcaacctgaagattttgcaacttactactgtcaacagagttacg
gtacccctccgtggacgttcggccaagggaccaaggtggagttcaaac
hcab4_K (SEQ ID NO: 86):
gacatccagatgacccagtctccatcctccctgtctgcatctgtaggagacagagtcaccatcacttgccgggcaagtcagagcattagcat
ctttttaaattggtatcggcagaaaccagggaaagcccctgagctcctgatctatgctgcatccagtttgcaaagtggggtcccatcaaggt
tcagtggcagtggatctgggacacatttcactctcaccatcagcagtctgcaacctgaagattttgcatcttactactgtcatcagagttac
agtacccctcagacgttcggccaagggaccaaggtggaaatcaaac
hcab18_K (SEQ ID NO: 87):
gacatccagatgacccagtctccattctccctgtctgcatctgtaggagacagagtcaccatcaattgccgggcaagtcagaccattttcag
ctatttgaattggtatcaacagaaaccagggaaagcccctaagctcctgatctatggtgcatccagtttgcaaagtggggtcccatcaaggt
tcagtggcagtggatctgggacagatttcactctcaccatcagcagtctgcaacctgaagattttgcaacttactactgtcaacagagttac
agtccccctaatacgttcggccaagggaccaaggtggaaatcaaac
hcab14_K (SEQ ID NO: 88):
gacatccagatgacccagtctccatcctccctgtctgcatctgtaggagacagagtcaccatcacttgccgggcaagtctgaccattttcag
ctatttaaattggtatcagcagaaaccagggaaagcccctaagctcctgatctatggtgcatccactttgcacagtggggtcccatcaagg
ttcagtggcagtggatctgggacagatttcactctcaccatcagcagtctgcaacctgaagattttgcaacttactactgtcaacagagttt
cactgcccctcgtacgttcggccaagggaccaaggtggaaatcaaac
hcab35_K (SEQ ID NO: 89):
gacatccagatgacccagtctccatcctccctgtctgcatctgtaggagacagagtcaccatcacttgccgggcaagtcagaccattttcagc
tatttaaattggtatcagcagaatccagggaaagcccctaagctcctgatctattctgcatcccgtttgcaaagtggggtcccatcaaggtt
cagtggcagtggatctgggacagatttcactctcaccatcagcagtctgcaacctgaagattttgcaacttactactgtcaacagagttaca
gtacccctcgaacgttcggccaagggaccaaggtggaaatcaaac
hcab22_K (SEQ ID NO: 90):
gacatccatatgacccagtctccagtctccctgtctgcatctgtaggagacagagtcaccatcacttgccgggcaagtcagagcattagcag
gtatttaaattggtatcagcagaaaccagggaaagcccctaagctcctgatctatgccgcatccagtttgcaaagtggggtcccatcaaggtt
cagtggcagtggatctgggacagatttcactctcaccatcagcagtctgcaacctgaagattttgcaacttacttctgtcagcagagttaca
gtcccctgtggacgttcggccaagggaccaaggtggagatcaaac
hcab7_K (SEQ ID NO: 91):
gacatccagatgacccagtctccattctccctgtctgcatctgtaggagacagagtcaccatcacttgccgggcaagtcagagcattagcag
ctatttaaattggtatcagcagaaaccagggaaagcccctaagctcctgatctatgctgcatccagtttgcaaagtggggtcccatcaaggt
tcagtggcagtggatctgggacagatttcactctcaccatcagcagtctgcaacctgaagattttgcaacttactactgtcaacagagttac
agtaccctcttcactttcggccccgggaccaaagtggatatcaaag
hcab16_K (SEQ ID NO: 92):
gacatccatatgacccagtctccattctccctgtctgcatctgtaggagacagagtcaccatcacttgccgggcaagtcagaacattaacagg
tatttaaattggtatcagcagaaaccagggaaagcccctaagctcctgatctatgctgcatccagtttgcaaagtggggtcccatcaaggtt
cagtggcagtggatctgggacagatttcactctcaccatcagcagtctgcaacctgaagattttgcaacttattactgtcaacagagttac
agtacccctcacacttttggccaggggaccaagctggagatcaaac
hcab36_K (SEQ ID NO: 93):
gacatcgtgatgacccagtctccagactccctggctgtgtctctgggcgagcgggccaccatcaactgcaagtccagccagagtgttttata
caactccaacaataagtacttcttaggttggtaccagcagaaaccgggacagcctccaaagctgctcatttcctgggcatctacccgggaat
ccggggtccctgaccgattcagtggcagcgggtctgggacagatttcactctcaccatcagcagcctgcaggctgaagatgtggcagtttat
tattgtcagcaatattatactactcccctcactttcggcggagggaccagggtggagatcaaac
hcab44_K (SEQ ID NO: 94):
gaaatagtgatgacgcagtctccagccaccctgtctgtgtctccaggggaaagagccaccctctcctgcagggccagtcagagtattagca
gccacttagcctggtaccagcagaaacctggccaggctcccagactcctcatctatggtgcatccaccagggccactggtgtcccagccag
gatcagtggcagtgggtcggggacagacttcactctcaccatcagcagcctgcagtctgaagattttgcaatttattactgtcagcactata
ctaactggcctccgaggagattcactttcggccctgggaccaaactggatatcaaaa
hcab6_K (SEQ ID NO: 95):
gaaattgttatgacccagtctccattctccctgtctttgtccccaggggaaagagccaccctctcctgcagggccagtcagagtgttagcagg
tacttagcctggtaccaacagaagcctggccaggctcccaggctcctcatctatgatgcaagcaacagggccactggcgtcccagccagg
ttcagtggcagtgggtctgggacagacttcactctcaccatcagcagcctagagcctgaagattttgctctttattactgtcaccagagtaa
tcactggcctccgttcacttttggccaggggacgaagctggagatcaaac
hcab45_K (SEQ ID NO: 96):
gaaattgtgttgacacagtctccagccaccctgtctttgtctccaggggaaagagccaccctctcctgcagggccagtcagagtgttagcatc
tacttagcctggtaccaacagaaacctggccaggctcccaggctcctcatctatgatgcatccaacagggccactggcatcccagccaggtt
cagtggcagtgggtctgggacagacttcactctcaccatcagcagcctagagcctgaagatttcgcagtttattactgtcagcagcgtagcaa
ctggccctctttcggcggagggaccaaggtggagatgaaac
hcab48_K (SEQ ID NO: 97):
gaaattgtgttgacacagtctccagccaccctgtctttgtctccaggggaaagagccaccctctcctgcagggccagtcagagtgttagcag
ctacttagcctggtaccaacagaaacctggccaggctcccaggctcctcatctatgatgcatccaacagggccactggcatcccagccagg
ttcagtggcagtgggtctgggacagacttcactctcaccatcagcagcctagagcctgaagattttgcagtttattactgtcagcagcgtag
cacctggcctccgctcactttcggcggagggaccaaggtggagatcaaac
hcab24_K (SEQ ID NO: 98):
gaaattgtgttgacgcagtctccaggcaccctgtctttgtctccaggggaaagagccaccctctcctgcaggtctagtcagagcgtcctgcat
agatacgtagcctggtaccagcatagacctgtgcaggctcccaggctcctcatctatgctgcgtccaatagggccactggcatcccagaca
ggttcactggaggggggtctgggacagacttcactctcaccatcagcagactggagcctgaagattttgcagtgttttactgtcagcagtatg
gtagctcacctcccactttcggcggagggaccaaggtggagatcaaac
hcab65_K (SEQ ID NO: 99):
gaaattgtgttgacgcagtctccaggcaccctgtctttgtctccaggggaaagagccaccctctcctgcagggccagtcagagtgttcccag
gaattatataggttggttccagcagaaacctggccaggctcccaggctcctcatctatggtgcatccagcagggccgctggcttcccagaca
gattcagtggcagtgggtctgggacagacttcactctcaccatcaccagactggagcctgaagattttgcaatgtattactgtcaccagtat
gataggttaccgtacacttttggccaggggaccaagctggagatcaaac
hcab30_K (SEQ ID NO: 100):
gaaattgtgttgacgcagtctccaggcaccctgtctttgtctccaggggaaagagccaccctctcctgcagggccactcagtatgttagcagc
aatcacttagcctggtaccagcagaaacctggccaggctcccaggctcctcatgtatggtgcatccattagggccactggcatcccagaca
ggttcagtggcagtgggtctgggacagacttcactctcaccatcagcggactggagcctgaagactttgcagtttattactgtcaccagtatg
ccacctcaccgctcgctttcggcggagggaccagggtggagatcaaac
hcab71_K (SEQ ID NO: 101):
gaaattgttatgacccagactccattctccctgtctttgtctccaggggaaagagccaccctctcctgcagggccagtcagagtgttagcaac
aacttcttagcctggtaccagcagaaacctggccaggctcccaggctcctcatctatggtgcatccaccagggccactggcatcccagaca
ggttcggtggcagtgggtctgggacagacttcactctcaccatcggcagactggagcctgaagattttgcagtgtattactgtcaccagtatg
ctacctcaccccggacgttcggccaagggaccaaggtggaactcaaac
hcab1_K (SEQ ID NO: 102):
gaaattgttatgacccagtctccagtctccctgtctttgtctccaggggaaagagccaccctctcctgcagggccagtcagagtgttagcagc
agctacttagcctggtaccagcagaaacctggccaggctcccaggctcctcatctatgctgcatccagcagggccactggcatctcagata
ggttcagtggcagtgggtctgggacagacttcactctcaccatcaacagactggagcctgaagattttgtagtatattactgtcagcactat
ggtaactcaccctggacgttcggccaagggaccaaggtggaaatcaaac
hcab21_K (SEQ ID NO: 103):
gaaattgtgttgacgcagtctccaggcaccctgtctttgtctccaggggaaagagccaccctctcctgcagggccagtcagactgttaccag
cagctccttagcctggtaccagcagaaacctggccaggctcccaggctcctcatctatggtgcatccagcagggccactggcatcccagac
aggttcagtggcagtgggtctgggacagtcttcactctcaccatcagcagactggagcctgaagattttgcagtgtattactgtcagcagta
tggtatctcaccctggtcgttcggccaagggaccaaggtggaaatcaaac
hcab64_K (SEQ ID NO: 104):
gaaattgtgttgacgcagtctccaggcaccctgtctttgtctccaggggaaagagccaccctctcctgcagggccagtcagagtgttagcag
cagctacttagcctggtaccagcagaaacctggccaggctcccaggctcctcgtctatggtgcatccaccagggccactggcatcccagac
aggttcagtggcagtgggtctgggacagacttcactctcaccatcagcagactggagcctgaagattttgcagtgtattactgtcaccagta
tagtagctcattctggacgttcggccaagggaccaaggtggaaatcaaac
hcab55_K (SEQ ID NO: 105):
gaaattgtgttgacgcagtctccagtctccctgtctttgtctccaggggaaagagccaccctctcctgcagggccagtcagagtgttagcagc
aactacttagcctggtaccagctgaaacctggccgggctcccaggctcctcatctatggtgcatccagcagggcccctggcatcccagaca
ggttcagtggcagtgggtctgggacagacttcactctcaccatcagcagactggagcctgaagattttgcagtgtattactgtcagcagtatg
gtatctcatcctggacgttcggccaagggaccaaggtggaaatcaaac
hcab23_K (SEQ ID NO: 106):
gaaattgtgttgacgcagtctccagtctccctgtctttgtctccaggggaaagagccaccctctcctgcagggccagtcagagtgttaccagc
agctacttagcctggtaccagcagaaacctggccaggctcccaggctcctcatctatggtgcatccatcagggccactggcatcccagaca
ggttcagtggcagtgggtctgggacagacttcactctcaccatcagcagactggagcctgaagattttgcagtgtattactgtcagcagtatg
gtagctcacttttttttggccaggggaccaagctggagatcaaac
hcab20_K (SEQ ID NO: 107):
gaaattgttatgacccagtctccagtctccctgtctttgtctccaggagacagagccaccctctcctgcagggccagtcagagtgttagcagc
agatatatagcctggtaccagcacaaacctggccaggctcccaggctcctcatctatgatgcatccagcagggccaatggcatcccagaca
ggttcagtggcagtgggtctgggacagacttcactctcaccattagcagactggagcctgaagactttgcagtgtattactgtcagcagtatg
gtagttcacctctgtggacttttggccaggggaccaagctggagatcaaac
hcab67_K (SEQ ID NO: 108):
gaaattgtgttgacgcagtctccaggcaccctgtctttgtctccaggggaaagagccaccctctcctgcagggccagtcagagtattagcag
cggctacatagcctggtaccagcagaaacctggccaggctcccaggctcctcatctatggtgcatccagcagggccactggcatcccaga
caggttcagtggcagtgggtctgggacagacttcactctcaccatcagcagtctggagcctgaagattttgcagtttattactgtcagcagt
atggtagttcacctctgtggacttttggccaggggaccaagctggacatcaaac
Variablc Lambda Chains
hcab71_L (SEQ ID NO: 109):
caggctgtgctgactcaggagccctcactgactgtgtccccaggagggacagtcactctcacctgtggctccagcactggagctgtcacca
gtggtcattatacttactggttccagcagaggtctggccaagcccccaggacactgatttatgatacaaccaacaaacactcctggacccctg
cccggttctcaggctccctccttgggggcaaagctgccctgaccctttcgggtgcgcagcctgaggatgaggctgactattactgcttgctct
catatagtgatgtaccgtcaggggtgttcggcggagggaccaagctgaccgtcctag
hcab73_L (SEQ ID NO: 110):
caggctgtggtgactcaggagccctcactgactgtgtctccaggagggacagtcactctcacctgtggctccagcactggagctgtcacca
gtggtcattatccctactggttccagcagaagcctggccaagcccccaggacactgatttatgatacaaccaacaaacactcctggacccct
gcccggttctcaggctccctccttgggggcaaagctgccctgaccctttcgggtgcgctgcctgacgatgaggctgactattattgtttgct
ctcctatagtgatgctccgtcaggggtgttcggcggagggaccaagctgaccgtcctgg
hcab21_L (SEQ ID NO: 111):
cagtctgtgctgactcagccaccctcagtgtcagtggccccaggaaagacggccatgattacttgtgggggtaacagctttggaggtaaaa
gtgtgcactggtaccagcagaagccaggccaggcccctgtgttggtcatctattataatagcgaccggccctcagggatccctgagcgattc
tctggctccaactctgggagctcggccaccctgactatcagcagggtcgaagccggggatgaggccgactattactgtcaggtgtgggata
gtagtagtgctctcggggtgttcggcggagggaccaagctgaccgtcctcg
hcab44_L (SEQ ID NO: 112):
tcctatgtgctgactcaggcaccctcactgtcagtggccccaggaaagacggccaggattacctgtgggggaaacaacattggaagtaaaa
gtgtgcactggtaccagcagaagccaggcctggcccctgtattggtcatctattatcatagcgaccggccctcagggatccctgagcgattc
tctggctccatctctgggaacacggccaccctgaccatcagcagggtcgaagccggggatgaggccgactattactgtcaggtgtgggat
agtactaatgctctcggggtgttcggcggagggaccaagctgaccgtcctag
hcab27_L (SEQ ID NO: 113):
cagtctgtgctgactcagcctgcctccgtgtctgggtctcctggacagtcgatcaccatctcctgcactggaaccagcagtgacgttggtggt
tatgactatgtctcctggtaccaacagtacccaggcaaagcccccaaactcatgatttatgatgtcagtcagcggccctcaggggtttctaat
cgcttctctggctccaagtctggcaacacggcctccctgaccatctctgggctccaggctgaggacgaggctgattattactgcagctcata
tacaagcaccaccactctggtattcggcggagggaccaagttgaccgtcctcg
hcab11_L (SEQ ID NO: 114):
cagtctgtgctgactcagcctgcctccgtgtctgggtctcctggacagtcgatcaccatctcctgcactggaaccagcagtgacgttggtggt
tataactatgtctcctggtaccaacagcacccaggcaaagcccccaaactcatgatttatgatgtcagtaagcggccctcaggggtttctaa
tcgcttctctggctccaagtctggcaacacggcctccctgaccatctctgggctccaggctgaggacgaggctgattattactgcagttcat
atacaaacagcaatacttttgtcttcggaactgggaccagggtcaccgtcctag
hcab37_L (SEQ ID NO: 115):
cagtctgtgctgactcagcctgcctccgtgtctgggtctcctggacagtcgatcaccatctcctgcactggaaccagcagtgacgtgggtggt
tataactatgtctcctggtaccaacatttcccaggcaaagcccccaaactcattatttatgatgtcagtaagcggccctcaggggtttctaa
tcgtttctctggctccaagtctggcaacgcggcctccctgaccatctctgggctccaggctgaggacgaggctgattattactgcagctcat
atacaaggagctacacttgtgtcttcggaactgggaccaaggtcgccgtcctag
hcab68_L (SEQ ID NO: 116):
cagtctgtgctgactcagccaccctcagcgtctgggacccccgggcagagggtcaccatctcctgttccggaggcagctccaacatcgga
agagattatgtatactggtaccagcaggtcacaggaacggcccccaaactcctcatctatcggaatgatcagcggcccgcaggggtccctg
accgaatctctggctccaagtctggcacctcagcgtccctggccatcagtgggctccggtccgaggatgaggctgattattactgttcatcat
gggatgacagtctgaatggttgggtgttcggcggagggaccaagttgaccgtcctag
hcab66_L (SEQ ID NO: 117):
cagtctgtgctgactcagccaccctcagcgtctgggacccccgggcagagggtcatcatctcttgttccggaagcagctccaacatcggaa
gagagactgtaaactggtaccagcaggtccctggaacggcccccaaactcctcatctctggtaataatcagcggcccgcaggagtccctg
accgattctctggctccaagtctggcacctcagcctccctggccatcagtgggctccaatctgaggatgaggctgattattactgtgcatct
tgggatgacagtctgattggtccggtgttcggcggagggaccaagctgaccgtcctag
hcab22_L (SEQ ID NO: 118):
cagtctgtgctgactcagccaccctcagcgtctgggaccctcgggcagagggtcaccatctcttgttctggaagcagctccaacatcggaa
gttattctgttaactggtaccagcagctcccaggaacggcccccaaactcctcatctacaaggataatcagcggccctcaggggtccctgac
cgattctctggctccaggtctggcacctcagcctccctggccatcagtgggctccagtctgaggatgaggctgattattactgtgcggcatgg
gatgacagcctgaatggtcatgtggtgttcggcggagggaccaagctgaccgtcctag
hcab29_L (SEQ ID NO: 119):
cagtctgtgctgactcagccaccctcagcgtctgggaccctcgggcagagggtcaccatctcttgttctggaagcagctccaacatcggaa
gttactctgttaactggtaccagcagctcccaggaacggcccccaaactcctcatctacaacgataatcagcggccctcaggggtccctgac
cgattctctggctccaggtctggcacctcagcctccctggccatcagtgggctccagtctgaggatgaggctgactattactgtgcagcatgg
gatgacagcctgaatggtcatgtggtgttcggcggagggaccaagctgaccgtcctag
hcab26_L (SEQ ID NO: 120):
cagtctgtgctgactcaggcaccctcagcgtctgggacccccggccagagggtcaccatctcttgttctggaagcaggtccaacatcggaa
gtaatactgtaaactggtaccagcagctcccaggaacggcccccaaactcctcatttatagtaatgatcagcggccctcaggggtccctgac
cgattctctggctccaagtctggcacctcagcctccctggccatcagtgggctccagtctgaggatgaggctgattattactgtgcagcatgg
gatgacagcctgaatgagtggctgttcggcggagggaccaagctgaccgtcctag
hcab61_L (SEQ ID NO: 121):
cagtctgtgctgactcagccaccctcagcgtctgggacccccgggcagagggtcaccatctcttgttctggaagcagctccaacatcggaa
agtttactgcaaactggtaccagcagctcccaggaacggcccccaaactcctcatctatagtaataatcagcggccctcaggggtccctgac
cgattctctggctccaagtctggcacctcagcctccctggccatcactgggctccagtctgaagatgaggctgattattactgtgcagcatgg
gatgacagcctgaatggtcgggtgttcggcggagggaccaagttgaccgtcctag
hcab15_L (SEQ ID NO: 122):
cagtctgtgctgacccagccgccctcagtgtctggggccccagggcagagggtcaccatttcctgcattgggagcagctccaacatcggg
gcaggttatgatgtccactggtaccagcaacttcctggaagagcccccaaactcctcatccacggtaacgccaatcggccctcaggggtcc
ctgaccgattctctggctccaagtctggcacctcagcctccctggccatcactggcctccaggctgaggatgaggctgattattactgccagt
cctatgacaacagcctgagaggttcgaaggttttcggcggagggaccaaggtgaccgtcctga
hcab51_L (SEQ ID NO: 123):
cagtctgtgctgactcagccgccctcggtgtctggggccccagggcagagggtcaccatttcctgcactgggagcagctccagcatcggg
gcaggttacgatctgcactggtaccagcaggttccaggaacagccccgagactcctcatctataataacaataatcggccctcaggggtcc
ctgaccgattctctggctccaagtctggcacctcagcctccctggccatcactgggctccaggctgaggatgaggctgattattactgccagt
cctatgacagcggcctgaatggttatgtcttcggaactgggaccagggtcaccgtcctgg

In certain embodiments, the bNAbs are human monoclonal antibodies or humanized monoclonal antibodies. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity. Non-human mammalian antibodies or animal antibodies can be humanized. The antibodies or monoclonal antibodies according to the disclosure may be humanized versions of for instance rodent antibodies or rodent monoclonal antibodies. Humanization of antibodies entails recombinant DNA technology, and is departing from parts of rodent and/or human genomic DNA sequences coding for H and L chains or from cDNA clones coding for H and L chains. Techniques for humanization of non-human antibodies are known to the skilled person as these form part of the current state of the art.

In certain embodiments, the neutralizing antibodies may comprise: a single-chain variable fragment (scFv) derived from a monoclonal antibody; a natural ligand of the target antigen; a peptide with sufficient affinity for the target; a single domain binder such as a camelid; an artificial binder single as a Darpin.

In certain embodiments, the neutralizing antibodies are bispecific antibodies, which specifically bind to two different epitopes or antigenic variants of the E2 protein.

In certain embodiments, the antibodies include polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V) regions capable of specifically binding the antigen, single chain antibody fragments, including single chain variable fragments (scFv), and single domain antibodies (e.g., sdAb, sdFv, nanobody) fragments. The term “antibody” encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the heavy and light chains of an antibody can be full-length or can be an antigen-binding portion (a Fab, F(ab′)₂, Fv or a single chain Fv fragment (scFv)). In other embodiments, the antibody heavy chain constant region is chosen from, e.g., IgG1, IgG2, IgG3, IgG4, IgM, IgAQ1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. Examples of antibody fragments include but are not limited to Fv, Fab, Fab′, Fab′-SH, F (ab)₂; diabodies; linear antibodies; variable heavy chain (Vu) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs. Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

In certain embodiments, the neutralizing antibodies comprises one or more mutations. These include deletions, substitutions, reversions to a genomic sequence, insertions. In certain embodiments, the neutralizing antibodies comprise one or more modified or unnatural amino acids, synthetic or derivatives of amino acids, e.g. glycosylated.

### Vectors

In certain embodiments, an expression vector comprises (i) a variable heavy chain sequence having at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 1-57 or 58; and either (ii) a variable kappa light (Vκ) chain sequence having at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 59-107 or 108; or (iii) a variable lambda light (Vλ) chain sequence having at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 109-122 or 123, or combinations thereof. In certain embodiments, (i) the variable heavy chain (VH) sequence comprises SEQ ID NOs: 1-57 or 58; (ii) the variable kappa light (Vκ) chain sequence comprises SEQ ID NOs: 59-107 or 108; or (iii) the variable lambda light (Vλ) chain sequence comprises SEQ ID NOs: 109-122 or 123, or combinations thereof.

In another embodiment, an expression vector comprises an isolated variable heavy chain (V_{H}) sequence comprising at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 1-57 or 58.

In another embodiment, an expression vector comprises an isolated variable kappa light (V_{κ}) chain sequence comprising at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 59-107 or 108.

In another embodiment, an expression vector comprises an isolated variable lambda light (Vλ) chain comprising at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NOs: 109-122 or 123.

In various embodiments, a vector or genetic construct is selected from the group comprising adenovirus, adeno-associated virus (AAV), herpes simplex virus, lentivirus, gammaretrovirus, retrovirus, alphavirus, flavivirus, rhabdovirus, measles virus, Newcastle disease virus, poxvirus, vaccinia virus, modified Ankara virus, vesicular stomatitis virus.

Vectors can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2p plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al., Bio Techniques, 34:167-171 (2003). A large variety of such vectors are known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D. T., et al. PNAS 88:8850-8854, 1991).

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.: 90 7603 (1993); Geller, A. L., et al., Proc Natl. Acad. Sci USA: 87:1149 (1990)], Adenovirus Vectors [LeGal LaSalle et al., Science, 259:988 (1993); Davidson, et al., Nat. Genet. 3:219 (1993); Yang, et al., J. Virol. 69:2004 (1995)] and Adeno-associated Virus Vectors [Kaplitt, M. G., et al., Nat. Genet. 8:148 (1994)].

When taken up by a cell, the genetic construct which includes the nucleotide sequence encoding the desired protein operably linked to the regulatory elements may remain present in the cell as a functioning extrachromosomal molecule or it may integrate into the cell's chromosomal DNA. DNA may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be administered to the cell. It is also contemplated to provide the genetic construct as a linear minichromosome including a centromere, telomeres and an origin of replication.

The regulatory elements necessary for gene expression of a DNA molecule include: a promoter, an initiation codon, a stop codon, and a polyadenylation signal. In addition, enhancers are often required for gene expression. It is necessary that these elements be operable linked to the sequence that encodes the desired proteins and that the regulatory elements are operably in the individual to whom they are administered.

Initiation codons and stop codon are generally considered to be part of a nucleotide sequence that encodes the desired protein. However, it is necessary that these elements are functional in the individual to whom the gene construct is administered. The initiation and termination codons must be in frame with the coding sequence.

Promoters and polyadenylation signals used must be functional within the cells of the individual. Examples of promoters useful to practice the present disclosure, especially in the production of a genetic vaccine for humans, include but are not limited to promoters from Simian Virus 40 (SV40, Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metalothionein.

In certain embodiments, a promoter comprises a MNDU3 promoter, a PGK promoter or the combination thereof.

Examples of polyadenylation signals useful to practice the present disclosure, especially in the production of a genetic vaccine for humans, include but are not limited to SV40 polyadenylation signals and LTR polyadenylation signals.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV. Genetic constructs can be provided with mammalian origin of replication in order to maintain the construct extrachromosomally and produce multiple copies of the construct in the cell. Plasmids pCEP4 and pREP4 from Invitrogen (San Diego, Calif.) contain the Epstein Barr virus origin of replication and nuclear antigen EBNA-1 coding region which produces high copy episomal replication without integration.

### Compositions

In certain embodiments, the bNAb composition is a vaccine composition. Such vaccine composition may be a prophylactic vaccine composition or a therapeutic vaccine composition. In particular the vaccine compositions can be applied for passive immunization. The insensitivity of the neutralizing anti-HCV antibodies of the invention and/or active fragments broadly neutralizing antibodies neutralize a plurality of HCV genotypes and decreases the chance that HCV viral mutants evolve (due to immune pressure) that can escape from the passive immunization with the bNAbs and/or active fragments thereof.

A “carrier”, or “adjuvant”, in particular a “pharmaceutically acceptable carrier” or “pharmaceutically acceptable adjuvant” is any suitable excipient, diluent, carrier and/or adjuvant which, by themselves, do not induce the production of antibodies harmful to the individual receiving the composition nor do they elicit protection. Preferably, a pharmaceutically acceptable carrier or adjuvant enhances the immune response. elicited by an antigen. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles; aluminum hydroxide, aluminum phosphate (see International Patent Application Publication No. WO93/24148), alum (KAI (SO₄)₂·12H₂O), or one of these in combination with 3-0-deacylated monophosphoryl lipid A (see International Patent Application Publication No. WO93/19780); N-acetyl-muramyl-L-threonyl-D-isoglutamine (see U.S. Pat. No. 4,606,918), N-acetyl-normuramyl-L-alanyl-D-isoglutamine, N-acetylmuramyl-L-alanyl-D-isoglutamyl-L-alanine2-(1′,2′-dipalmitoyl-sn-glycero-3-hydroxyphosphoryloxy)ethylamine; RIBI (ImmunoChem Research Inc., Hamilton, Mont., USA) which contains monophosphoryl lipid A (i.e., a detoxified endotoxin), trehalose-6,6-dimycolate, and cell wall skeleton (MPL+TDM+CWS) in a 2% squalene/Tween 80 emulsion. Any of the three components MPL, TDM or CWS may also be used alone or combined 2 by 2; adjuvants such as Stimulon (Cambridge Bioscience, Worcester, Mass., USA), SAF-1 (Syntex); adjuvants such as combinations between QS21 and 3-de-O-acetylated monophosphoryl lipid A (see International Patent Application Publication No. WO94/00153) which may be further supplemented with an oil-in-water emulsion (see, e.g., International Patent Application Publication Nos. WO95/17210, WO97/01640 and WO9856414) in which the oil-in-water emulsion comprises a metabolisable oil and a saponin, or a metabolisable oil, a saponin, and a sterol, or which may be further supplemented with a cytokine (see International Patent Application Publication No. WO98/57659); adjuvants such as MF-59 (Chiron), or poly [di(carboxylatophenoxy)phosphazene] based adjuvants (Virus Research Institute); blockcopolymer based adjuvants such as Optivax (Vaxcel, Cytrx) or inulin-based adjuvants, such as Algammulin and GammaInulin (Anutech); Complete or Incomplete Freund's Adjuvant (CFA or IFA, respectively) or Gerbu preparations (Gerbu Biotechnik). It is to be understood that Complete Freund's Adjuvant (CFA) may be used for non-human applications and research purposes as well; a saponin such as QuilA, a purified saponin such as QS21, QS7 or QS 17, β-escin or digitonin; immunostimulatory oligonucleotides comprising unmethylated CpG dinucleotides such as [purine-purine-CG-pyrimidine-pyrimidine] oligonucleotides. These immunostimulatory oligonucleotides include CpG class A, B, and C molecules (Coley Pharmaceuticals), ISS (Dynavax), Immunomers (Hybridon). Immunostimulatory oligonucleotides may also be combined with cationic peptides as described, e.g., by Riedl et al. (2002); Immune Stimulating Complexes comprising saponins, for example Quil A (ISCOMS); excipients and diluents, which are inherently non-toxic and non-therapeutic, such as water, saline, glycerol, ethanol, wetting or emulsifying agents, pH buffering substances, preservatives, and the like; a biodegradable and/or biocompatible oil such as squalane, squalene, eicosane, tetratetracontane, glycerol, peanut oil, vegetable oil, in a concentration of, e.g., 1 to 10% or 2.5 to 5%; vitamins such as vitamin C (ascorbic acid or its salts or esters), vitamin E (tocopherol), or vitamin A; carotenoids, or natural or synthetic flavonoids; trace elements, such as selenium; any Toll-like receptor ligand.

More in particular for the antibodies of the disclosure, a “carrier”, or “adjuvant”, or “diluent” in particular a “pharmaceutically acceptable carrier” or “pharmaceutically acceptable adjuvant” or “pharmaceutically acceptable vehicle” is any suitable excipient, diluent, carrier, adjuvant, and/or vehicle which, by themselves, do not induce harmful effects to the individual receiving the composition nor do they elicit protection. Preferably, a pharmaceutically acceptable carrier, adjuvant or vehicle enhances or conserves the activity of the vaccine by buffering, stabilizing, protecting from chemical modification, degradation or aggregation, or controlling the release of the anti-HCV antibody and/or the active fragment thereof. Suitable excipient, diluent, carrier, adjuvant, and/or vehicle typically comprise one or more of the compounds included in the following non-exhaustive list: large slowly metabolized macromolecules such as proteins, polysaccharides, polylactic acids, polyglycolic acids, polymeric amino acids, amino acid copolymers and poly-ethylene glycols; excipients and diluents, which are inherently non-toxic and non-therapeutic, such as water, saline, glycerol, ethanol, EDTA, wetting or emulsifying agents, pH buffering substances, preservatives, detergents and more particularly non-ionic detergents such as polysorbate, sugars such as trehalose, sucrose, mannitol, and the like; vitamins such as vitamin C (ascorbic acid or its salts or esters), vitamin E (tocopherol), or vitamin A; carotenoids, or natural or synthetic flavonoids; trace elements, such as selenium.

A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles.

Typically, a vaccine or vaccine composition is prepared as an injectable, either as a liquid solution or suspension. Injection may be subcutaneous, intramuscular, intravenous, intraperitoneal, intrathecal, intradermal, intraepidermal. Other types of administration comprise implantation, suppositories, oral ingestion, enteric application, inhalation, aerosolization or nasal spray or drops. Solid forms, suitable for dissolving in, or suspension in, liquid vehicles prior to injection May also be prepared. The preparation may also be emulsified or encapsulated in liposomes for enhancing adjuvant effect.

An effective amount of an active substance in a vaccine or vaccine composition is the amount of said substance required and sufficient to elicit an active immune response or the amount of said substance required and sufficient to result in effective passive immunization. It will be clear to the skilled artisan that an active immune response sufficiently broad and vigorous to provoke the effects envisaged by the vaccine composition may require successive (in time) immunizations with the vaccine composition as part of a vaccination scheme or vaccination schedule. Likewise, to provoke the effects envisaged by passive immunization, the vaccine composition may require successive (in time) immunizations with the vaccine composition as part of a vaccination scheme or vaccination schedule. The “effective amount” may vary depending on the health and physical condition of the individual to be treated, the age of the individual to be treated (e.g. dosing for infants may be lower than for adults) the taxonomic group of the individual to be treated (e.g. human, nonhuman primate, primate, etc.), the capacity of the individual's immune system to mount an effective immune response (in case of active immunization), the degree of protection desired, the formulation of the vaccine, the treating doctor's assessment, the strain and load of the infecting pathogen and other relevant factors. It is expected that the effective amount of the anti-HCV antibodies of the invention will fall in a relatively broad range that can be determined through routine trials. The amount can vary from 0.01 to 1000 μg/dose, more particularly from 0.1 to 100 μg/dose. Usually, however, this amount will vary from 0.1 to 100 mg/kg/dose, more particularly from 0.5 to 20 mg/kg/dose. Dosage treatment may be a single dose schedule or a multiple dose schedule. Dosage may also be adapted such that occurrence of the prozone effect is prevented.

### General Methods

It will be understood that monoclonal antibodies binding to Hepatitis C virus will have several applications. These include the production of diagnostic kits for use in detecting and diagnosing Hepatitis C virus infection, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce Hepatitis C virus-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984). HMMA2.5 cells or MFP-2 cells are particularly useful examples of such cells. Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10-6 to 1×10-8, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 104 times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates. B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248:443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267:252-259; Engen and Smith (2001) Anal. Chem. 73: 256A-265A. When the antibody neutralizes Hepatitis C virus, antibody escape mutant variant organisms can be isolated by propagating Hepatitis C virus in vitro or in animal models in the presence of high concentrations of the antibody. Sequence analysis of the Hepatitis C virus gene encoding the antigen targeted by the antibody reveals the mutation(s) conferring antibody escape, indicating residues in the epitope or that affect the structure of the epitope allosterically.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference anti-Hepatitis C virus antibody, the above-described binding methodology is performed in two orientations: In a first orientation, the reference antibody is allowed to bind to the Hepatitis C virus antigen under saturating conditions followed by assessment of binding of the test antibody to the Hepatitis C virus antigen. In a second orientation, the test antibody is allowed to bind to the Hepatitis C virus antigen under saturating conditions followed by assessment of binding of the reference antibody to the Hepatitis C virus antigen. If, in both orientations, only the first (saturating) antibody is capable of binding to the Hepatitis C virus antigen, then it is concluded that the test antibody and the reference antibody compete for binding to the Hepatitis C virus antigen. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, there are provided monoclonal antibodies having clone-paired CDRs from the heavy and light chains as illustrated in Tables 3 and 4, respectively. Such antibodies may be produced by the clones discussed below in the Examples section using methods described herein.

In another aspect, the antibodies may be defined by their variable sequence, which include additional “framework” regions. These are provided in Tables 1 and 2 that encode or represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing applies to the nucleic acid sequences comprising SEQ ID NOs: 1-123.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection. One particular example of algorithms that are suitable for determining percent sequence identity and sequence similarity are the BLAST and BLAST 2.0 algorithms, which are described in Altschul et al. (1977) Nucl. Acids Res.25:3389-3402 and Altschul et al. (1990) J. Mol. Biol.215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) J. Biol. Chem. 277 (30): 26733-26740; Davies J. et al. (2001) Biotechnology & Bioengineering 74 (4): 288-294). Methods of altering carbohydrate contents are known to those skilled in the art, see, e.g., Wallick, S. C. et al. (1988) J. Exp. Med. 168 (3): 1099-1109; Tao, M. H. et al. (1989) J. Immunol. 143 (8): 2595-2601; Routledge, E. G. et al. (1995) Transplantation 60 (8): 847-53; Elliott, S. et al. (2003) Nature Biotechnol. 21:414-21; Shields, R. L. et al. (2002) J. Biol. Chem. 277 (30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody. C. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mY) outperforms several other nucleoside modifications and their combinations in terms of translation capacity. In addition to turning off the immune/eIF2a phosphorylation-dependent inhibition of translation, incorporated N1mY nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F (ab\hat{ }) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101 (incorporated herein by reference) states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5=1), alanine (−0.5), and glycine (0); hydrophobic, aromatic amino acids: tryptophan (−3.4), phenylalanine (−2.5), and tyrosine (−2.3). It is understood that an amino acid can be substituted for another having a similar hydrophilicity and produce a biologically or immunologically modified protein. In such changes, the substitution of amino acids whose hydrophilicity values are within +2 is preferred, those that are within +1 are particularly preferred, and those within +0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG1 can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcgR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.). For example, one can generate a variant Fc region of an antibody with improved C1q binding and improved FcgRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described. High resolution mapping of the binding site on human IgG1 for FcgRI, FcgRII, FcgRIII, and FcRn and design of IgG1 variants with improved binding to the FcgR (Shields et al., 2001, J. Biol. Chem. 276: 6591-6604). A number of methods are known that can result in increased half-life (Kuo and Aveson, (2011)), including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified, either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

The modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcgRI, FcgRII and FcgRIIIa. The variant and unmodified recombinant mAbs were compared for their capacity to neutralize and enhance infection by the four dengue virus serotypes. LALA variants retained the same neutralizing activity as unmodified mAbs, but were completely devoid of enhancing activity. LALA mutations of this nature are therefore contemplated in the context of the presently disclosed antibodies.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region. Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, GIF, G2F, GNF, GNGNF or GNGNFX containing glycoforms. In one embodiment of the present disclosure, the antibody dissociates from Fc gamma RI with a Kd of 1×10-8 M or less and from Fc gamma RIII with a Kd of 1×10-7 M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication 20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Antibodies can be engineered for enhanced biophysical properties. One can use elevated temperature to unfold antibodies to determine relative stability, using average apparent melting temperatures. Differential Scanning calorimetry (DSC) measures the heat capacity, Cp, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, CH2, and CH3 domains). Typically unfolding of the Fab domain produces the strongest peak. The DSC profiles and relative stability of the Fc portion show characteristic differences for the human IgG1, IgG2, IgG3, and IgG4 subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

One can determine the intrinsic solubility score of antibody sequences. The intrinsic solubility scores can be calculated using CamSol Intrinsic (Sormanni et al., J Mol Biol 427, 478-490). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366:449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Generally, it is thought that autoreactive clones should be eliminated during ontogeny by negative selection, however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

### EXAMPLES

### Example 1: Convergent Evolution of Human HCV bNAb Lineages with Diverse V_{H} Genes

Broadly-neutralizing antibodies (bNAbs) are associated with natural clearance of human hepatitis C virus (HCV) infection, informing vaccine development. To date, most HCV-reactive human bNAbs target the envelope glycoprotein 2 (E2) front-layer and utilize the V_{H}1-69 gene segment, suggesting that bNAb induction is genetically restricted.

Here, a mixture of genetically and antigenically diverse E2 ectodomain proteins were used to isolate E2-reactive B cells from the longitudinally-collected blood of an Elite Neutralizer with broadly neutralizing plasma, previously demonstrated as having antibody-mediated clearance of infection (Kinchen et al., 2018b). B cell receptor sequencing (BCR-seq) was performed at timepoints from initial infection through viral clearance, monoclonal B cell cultures were characterized, E2-reactive mAbs were isolated, and bNAbs were identified. V and D-gene usage of these bNAbs were characterized and their epitopes on E2 were mapped, and novel neutralizing epitopes were identified in the back layer of E2. The structures of representative E2-bNAb complexes, including the first structures of non-V_{H}1-69 bNAb-E2 complexes were solved. Evidence of early emergence of bNAbs and ongoing maturation over time was found, identifying convergent evolution of multiple bNAb lineages using diverse V_{H} and D-gene segments. Overall, these studies highlight the remarkable diversity and plasticity of the human bNAb response against HCV, while identifying key bNAb-E2 interactions associated with broad neutralizing activity and natural clearance of HCV infection.

### Materials and Methods

### Study Subjects

Baltimore Before and After Acute Study of Hepatitis (BBAASH) is a prospective cohort (Cox et al., 2005b, Burke et al., 2012, Chattergoon et al., 2014, Cox et al., 2005a, Osburn et al., 2010) of people who inject drugs in Baltimore who are followed from before the time they are infected with HCV, through spontaneous clearance/persistence of HCV. These identity-unlinked samples are stored under code in aliquots in −80 freezers (plasma) or liquid nitrogen (PBMC) with continuous temperature monitoring. HCV viral loads (IU/mL) were quantified after RNA extraction using Abbot real-time HCV PCR. The study was approved by the Institutional Review Board of Johns Hopkins Hospital and informed written consent was obtained from all study participants.

### Cell Lines

Huh7 cell line was obtained from Dr. Charles Rice, Rockefeller University, New York, USA. HEK-293T-CD81 knockout cells were obtained from Dr. Joe Grove, University of Glasgow, United Kingdom. HEK293-T cell line was obtained from ATCC, and Expi293F from ThermoFischer.

### Soluble E2 Variants Used for B Cell Staining

Genes encoding 1a157, 1b09, and 1b21 E2 ectodomains, residues 384-643 (numbering based on strain H77), were cloned into a mammalian expression vector pHCMV3 that includes an N-terminal Ig-kappa secretion signal and expressed by transient transfection of HEK293T cells. As previously described (Ogega et al., 2022), these 3 full length, unmutated E2 ectodomains were selected from a library of 89 distinct genotype 1 HCV E1E2 variants (E1-Diwany et al., 2017).

### Cell Staining and Flow Cytometry

As previously described (Ogega et al., 2022), PBMCs were stained with a mixture of the 3 E2 proteins. Briefly, following incubation and blocking by anti-CD81 antibody (BD Cat #555675) and Fc blocker (BD Cat #564220) in FACS Buffer (1×PBS with 1% BSA), the PBMCs were coated with the E2 mixture at 5 μg/mL. Next, the PBMCs were incubated with propidium iodide (PI), CD10-PE, CD19-BV421, CD3-APC H7, IgM-BB515, IgD-BB515, and Anti His-A647 before sorting E2-reactive class-switched memory B cells using a MoFlo flow cytometer (BD).

### Sorted E2-Reactive B Cell Cultures and ELISAs

Following previous methods (Huang et al., 2013a, Huang et al., 2013b, Ogega et al., 2022), sorted memory B cells were seeded at 1, 2, 4, and 20 B cells per well in clear u-bottom 96-well cell culture plates along with irradiated mouse 3T3 fibroblast cells expressing CD40L, IL-2 (Fisher Scientific Cat #202IL050CF), and IL-21 (Fisher Scientific Cat #8879IL050). 3T3-CD40L cells were obtained through the NIH AIDS Reagent Program, Division of AIDS, NIAID, NIH: Cat #12535 3T3-msCD40L cells, from Dr. Mark Connors. On day 14, supernatants were isolated from the cultures, and cells were stored in-80 with lysis buffer.

For IgG ELISAs, 96-well Maxisorp plates were coated with Jackson Alkaline Phosphatase AffiniPure Goat Anti-Human IgG, Fcγ fragment specific (Fisher Scientific Cat #109-055-098). Following incubation with culture supernatants, Goat Anti-Human IgG Fab Secondary Antibody (MyBioSource Cat #MBS8216436) was used at a dilution of 1:4,000, with alkaline phosphatase yellow (pNPP) for readout.

For E2 antigen-specific ELISAs, Immulon 2b microtiter plates were coated with Lectin followed by incubation with E2 antigen overnight. Plates were blocked with PBS-TMG (PBS+0.5% tween 20±1% non-fat dry milk+1% goat serum) and then incubated with B cell culture supernatants or mAbs. Anti-human IgG-HRP (BD-Pharmingen Cat #555788) was used at a 1:4,000 dilution and TMB peroxidase substrate to develop with IN sulfuric acid used to stop the reaction.

### Monoclonal Antibody Expression

RNA was extracted from the cells with IgG of interest using the RNeasy Micro Kit (Qiagen) by the manufacturer's instructions. The RNA was reverse transcribed by random hexamers using SuperScript IV Reverse Transcriptase (ThermoFisher) by the manufacturer's instructions. Platinum Taq DNA Polymerase High Fidelity (ThermoFisher) was used to perform heavy and light chain nested PCR reactions in 96-well plates containing 20 nm each primer or primer mix as described by Tiller et al. (Tiller et al., 2008). PCR products were agarose gel purified and sanger sequenced.

Sequences were synthesized (Twist Biosciences) and cloned into a human IgG1, IgK, or IgL expression plasmids (pTwist CMV BetaGlobin WPRE Neo_IgG1Fc, pTwist CMV BetaGlobin WPRE Neo_Kappa_TAG or pTwist CMV BetaGlobin WPRE Neo_Lambda_TAG). Expi293 cells were transfected with the heavy and light chain plasmids using the Gibco Expi293 Expression System (Thermo Fisher Scientific). Monoclonal immunoglobulin proteins were harvested and purified using Pierce™ Protein G Agarose (Cat. #20398) and concentrated using Pierce™ Protein Concentrator PES, 30K MWCO (Cat. #88529).

### B Cell Receptor Sequencing

Sorted cells were lysed and RNA was isolated using the RNeasy Micro Kit (Qiagen) as described above. Briefly, cells in lysis buffer containing 2-mercaptoethanol were combined with 70% ethanol and applied to a purification column. Following multiple washes and the application of DNAse, RNA was eluted in RNAse free water. RNA quality was verified using a 2100 Bioanalyzer (Agilent). cDNA libraries were produced using the SMARTer Human BCR IgG IgM H/K/L Profiling Kit (Takara) according to the manufacturer's instructions and with the addition of unique molecular identifiers (UMIs). AMPure XP beads (Beckman) were used for purification steps. All sequencing was performed on an Illumina MiSeq at a depth of 1 million reads. cDNA library quality was verified using a 2100 Bioanalyzer (Agilent) and quantified using a Qubit (ThermoFisher). Clonotypes were quantified using MiXCR software24. Clonotypes were defined using sequences with nucleotides with Phred quality scores >20, however subsequent sequences with nucleotides with lower quality scores could be matched to existing clonotypes if the number of low-quality nucleotides was <0.7%. All additional analysis was done in R Studio (version 4.0.2).

### HCVpp and Neutralization Assays

As described (Bailey et al., 2019), HIV group-specific antigen (Gag)-packaged HCVpps were produced by lipofectamine-mediated transfection of HCV E1/E2 expression plasmid, pNL4-3.Luc.R-E-expression plasmid containing the env-defective HIV proviral genome (National Institutes of Health AIDS Reagent Program), and pAdVantage (Promega, Madison, WI) plasmid into CD81-knockout HEK293T cells (Kalemera et al., 2021). The panel of 15 HCVpp as described by Salas et al. (Salas et al., 2021) plus an additional 2 Tier 4 resistant variants HCVpp (UKNP2.4.1 and UKNP6.1.1) were used in neutralization assays. Neutralization assays were performed as previously described (Bailey et al., 2019, Wasilewski et al., 2016). Briefly, purified mAbs at 100 μg/mL or heat-inactivated plasma samples at 1:100 dilution were incubated with HCVpp for 1 hour at 37° C. prior to addition to HuH7 cells in duplicate with nonspecific human isotype control. The percentage of neutralization was calculated as [1-(RLU_{mAb}/RLU_{hIgG})]×100, with the values averaged across 2 biological and 2 technical replicates. Based on 65 independent tests performed in duplicate with a variety of different HCVpp (Salas et al., 2021), nonspecific neutralization by isotype control mAb R04 at 100 mcg/mL was an average of 1.0%, with a standard deviation of 19.9%. Therefore, we set the cutoff for true-positive neutralization at >25%.

### ELISA Assays for Epitope Mapping

To determine E2 front layer (FRLY) or antigen site 412 (AS412) dependent binding, ELISA binding of each mAb was measured with wild-type 1a157 E2, wild-type 1a53 E2, 1a157 E2 FRLY KO or 1a53 E2 AS412 KO proteins. 1a157 E2 FRLY KO contains mutations T425A, L427A, N428A, S432A, G436A, W437A, G530A, and D535A that abrogate binding of known FRLY-reactive mAbs. 1a53 E2 AS412 KO contains mutations L413A, G418A, and W420A that abrogate binding of known AS412-reactive mAbs.

ELISA binding of each mAb was also measured with wild-type variant H77 E1E2 or 25 H77 E1E2 single alanine mutant variants, selected based on binding epitopes of all published bNAbs (a gift of Dr. Mansun Law at The Scripps Research Institute). The single substitutions were as follows: V246A, R259A, H316A, M323A, M324A, L413A, N417A, G418A, W420A, T425A, N428A, G436A, L441A, F442A, G523A, W529A, G530A, T534A, D535A, P545A, G547A, W549A, R639A, R657A, and D698A. E1E2 lysates were generated following lipofectamine transfection of HEK293T cells.

For all ELISAs, Immulon 2b microtiter plates were coated with GNA-lectin followed by coating with either 1a157, 1a157 FRLY KO, 1a53, 1a53 AS412 KO, H77, or H77 alanine mutant proteins. Purified hcab mAbs were tested at 10 μg/ml and those with higher affinity were titrated and then tested at ~their EC90 concentration (HEPC74, hcab27, hcab31, hcab48, and hcab60). Binding OD₄₅₀ was normalized for relative protein concentration of each sE2 protein using OD₄₅₀ of anti-HIS binding for each variant. mAbs were separated based on front layer affinity and then hierarchical clustering using Ward's minimum variance method in the hclust R package was used. An unrooted clustering tree was created with the ape R library, as previously described (Pierce et al., 2016).

### Expression and Purification of E2-Fab Complexes

Hcab55 Fab-1b09 E2ecto, hcab64 Fab-1b09 E2ecto, and hcab40 Fab-1b09 E2ecto complexes for structural studies were expressed by co-transfecting expression vectors encoding His-tagged Fab and untagged E2 and purifying Fab-E2 complexes from supernatants using Ni-NTA chromatography on HisTrap HP column (GE Healthcare) followed by SEC on a Superdex 200 Increase 10/300 GL column (GE Healthcare). Expi293F cells were grown in the presence of 5 mM kifunensine (Sigma) to express the hcab Fab-E2ecto complexes.

### Crystallization

Commercially-available screens from Hampton Research and Molecular Dimensions were used to screen initial crystallization conditions by vapor diffusion in sitting drops.

hcab55-E2ecto crystals were grown using 0.2 μL of the protein complex in TBS and 0.2 μL of mother liquor (0.2 M lithium citrate tribasic tetrahydrate and 20% PEG 3,350) and cryoprotected in mother liquor supplemented with 25% (v/v) glycerol. hcab64-E2ecto crystals were grown using 0.2 μL of the protein complex in TBS and 0.2 μL of mother liquor (8% Tacsimate, pH 7.0 and 20% PEG 3,350) and cryoprotected in mother liquor supplemented with 25% (v/v) glycerol. hcab40-E2ecto crystals were grown using 0.2 L of the protein complex in TBS and 0.2 μL of mother liquor (0.1 M Sodium malonate pH 6.0 and 12% PEG 3,350) and cryoprotected in mother liquor supplemented with 30% (v/v) glycerol.

X-ray diffraction data from cryopreserved crystals were collected at the Stanford Synchrotron Radiation Lightsource on beamline 12-2 using a PILATUS 6M detector. Images were processed and scaled using iMosflm (Battye et al., 2011) and Aimless as implemented in the CCP4 software suite (Evans and Murshudov, 2013). Structures were solved by molecular replacement using the HEPC74-Fab (PDB 6MEH) and 1b09 HCV E2ecto (PDB 6MEI) structures as search models. The models were refined and validated using Phenix.refine (Adams et al., 2010). Iterative manual model building and corrections were performed using Coot (Emsley and Cowtan, 2004). Glycans were initially interpreted and modeled using Fₒ-F_{c} maps calculated with model phases contoured at 2 s, followed by 2Fₒ-F_{c} simulated annealing composite omit maps generated in Phenix in which modeled glycans were omitted to remove model bias (Adams et al., 2010). The quality of the final models was examined using MolProbity (Chen et al., 2010).

Models were superimposed and FIGS. rendered using the PyMOL molecular visualization system (Version 2.1, Schrödinger, LLC). Buried surface areas (BSAs) were determined using the PDBePISA web-based interactive tool (Krissinel and Henrick, 2007). Potential hydrogen bonds were assigned using criteria of a distance of <4.0 Å and an A-D-H angle of >90°, and the maximum distance allowed for a van der Waals interaction was 4.0 Å. Rmsd calculations were done in PyMOL following pairwise Ca alignments without excluding outliers. Antibody residues were numbered according to the Kabat numbering scheme, and ImMunoGeneTics (IMGT) definitions of CDRs were used throughout the paper,

### Statistical Analysis

MiXCR software24, R studio (ape package), and Prism (Graphpad) software were used.

### Results

### Elite Neutralizer Plasma is Broadly Neutralizing Against Genetically and Antigenically Diverse HCVpp.

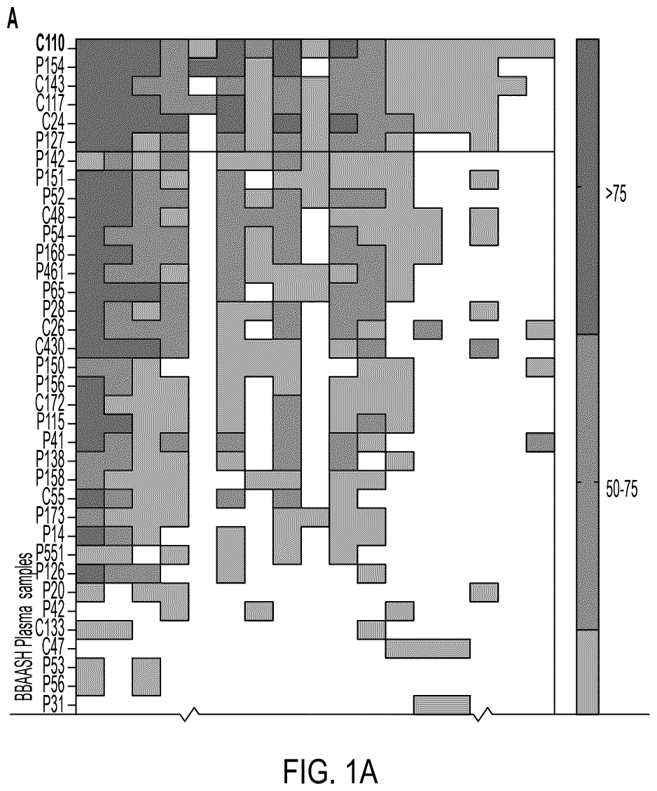

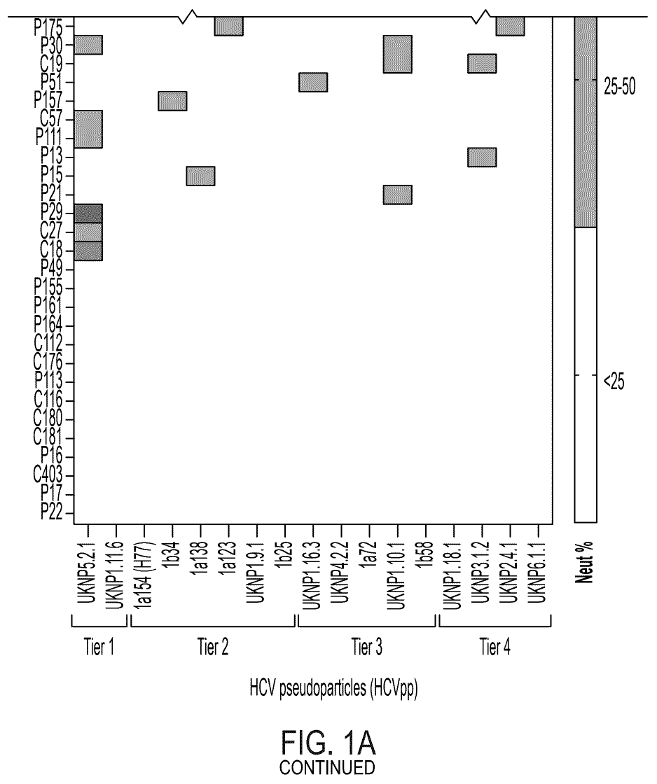

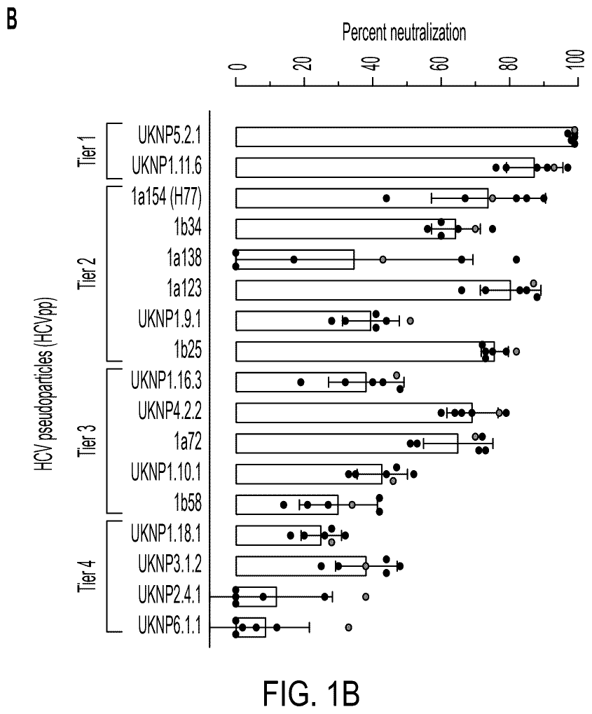

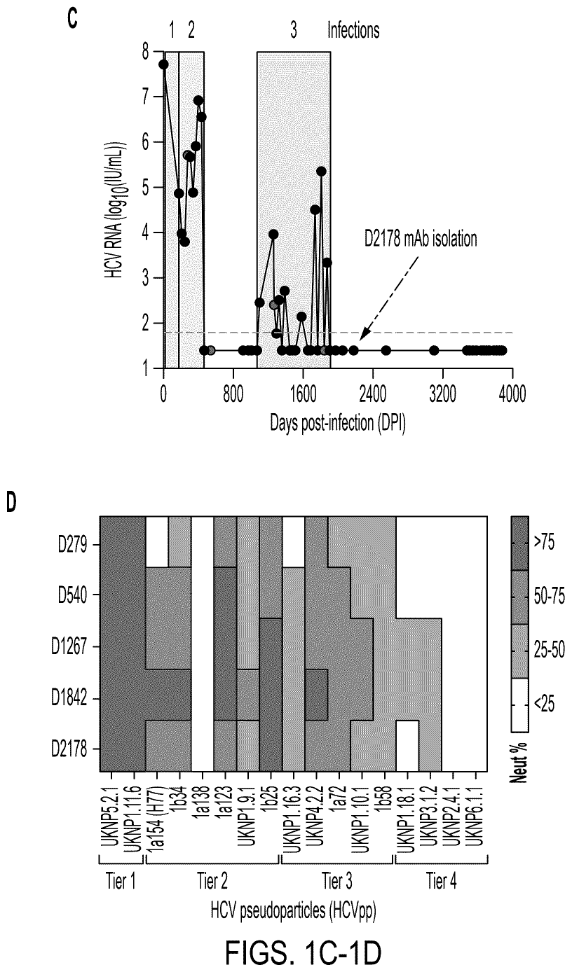

The Baltimore Before and After Acute Study of Hepatitis (BBAASH) is a prospective, longitudinal cohort of people who inject drugs, with monthly blood collection from before the time of infection through spontaneous viral clearance or the transition to chronic infection (Cox et al., 2005b). To identify a participant for exhaustive BCR repertoire analysis and mAb isolation, we screened neutralizing breadth of early-infection plasma samples from 63 BBAASH participants using a panel of 17 HCV pseudoparticles (HCVpp). Participants with clearance of infection (n=21) were sampled a median of 275 days post infection (dpi), while participants with persistent infection (n=42) were sampled a median of 238 dpi. The HCVpp panel consists of a recently described antigenically diverse set of 15 HCVpp spanning four tiers of increasing neutralization resistance (Salas et al., 2021), as well as 2 additional highly neutralization-resistant (Tier 4) variants (UKNP2.4.1 and UKNP6.1.1). A broad spectrum of neutralizing breadth was observed with no clear distinction between those with naturally cleared or persistent infection (FIG. 1A). 10% of participants with the greatest plasma neutralizing breadth were designated as ‘Elite Neutralizers’ (EN). Of interest, the subject with greatest neutralizing breadth among all EN, designated subject C110, naturally cleared three infections without treatment, and the inventors previously demonstrated that neutralizing antibodies played a direct role in clearance of their primary infection (Kinchen et al., 2018b). Plasma from C110 displayed a pattern of relative neutralizing activity across the HCVpp panel that was representative of the EN subset of BBAASH participants (FIG. 1B). Moreover, HEPC74, a bNAb that has been extensively characterized (Bailey et al., 2017, Flyak et al., 2018), was previously isolated from this subject by human hybridoma technology. To capture the full bNAb repertoire, a timepoint 2,178 dpi was selected for mAb isolation. By that time, C110 had cleared 3 infections without antiviral treatment. Additionally, 4 longitudinal timepoints throughout the course of infection were selected for BCR-seq of E2-reactive B cells to characterize bNAb induction and evolution (FIG. 1C). Plasma from all five timepoints displayed broad neutralizing activity, beginning early during primary infection (FIG. 1D), as has been shown for other cleared infections (Osburn et al., 2014). In summary, by screening the BBAASH cohort with a diverse panel of 17 HCVpp, we identified an EN with spontaneous clearance of multiple HCV infections and broadly neutralizing plasma representative of other EN in the cohort.

### Increased Somatic Mutation and Longer CDRH3 in E2-Reactive BCRs.

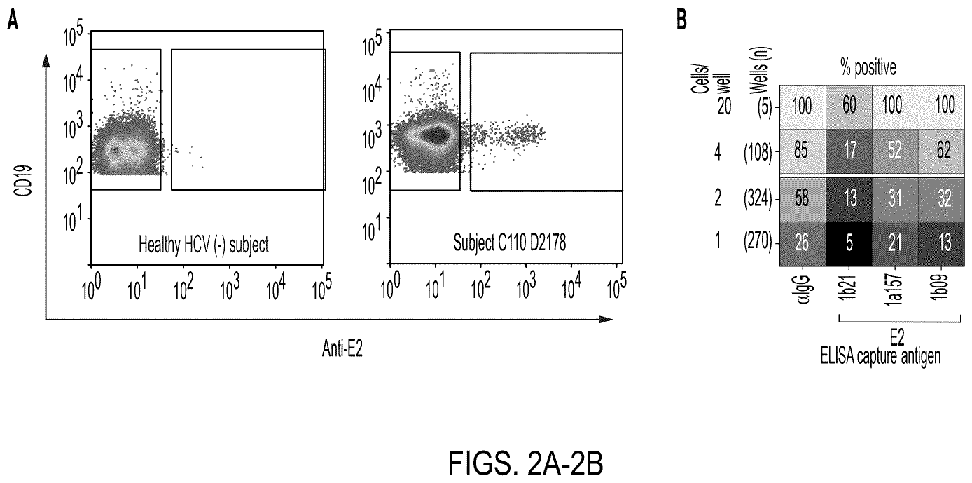

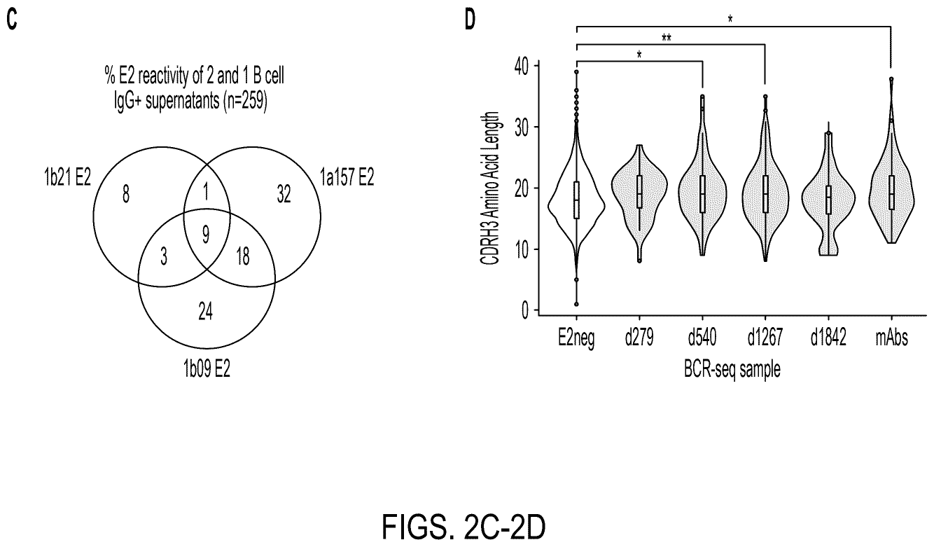

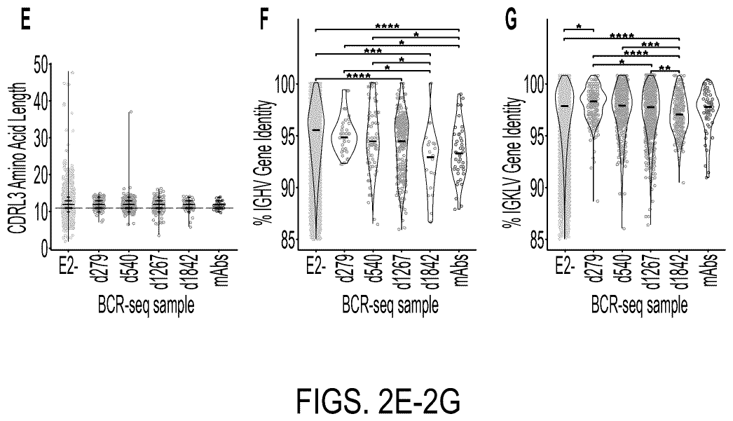

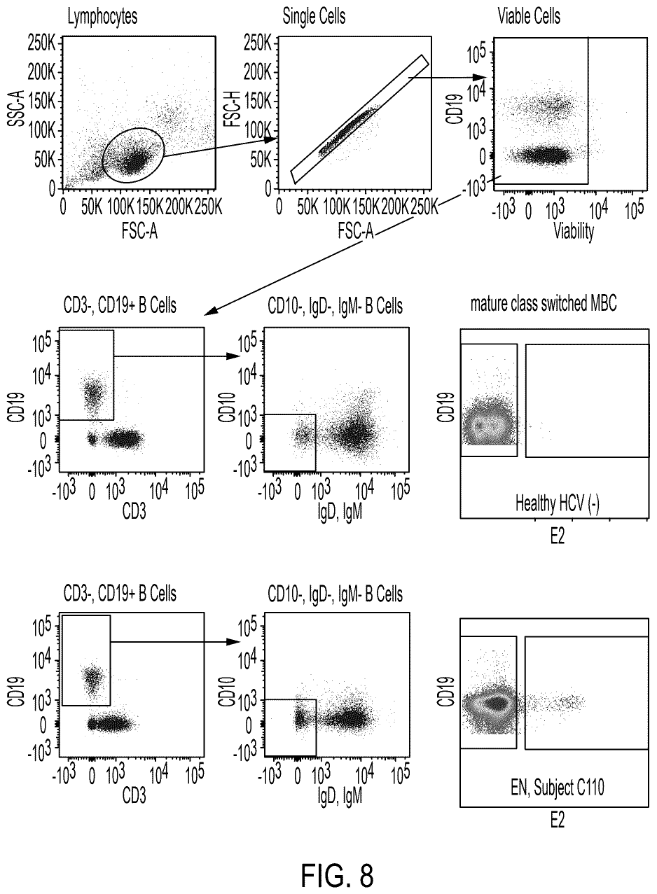

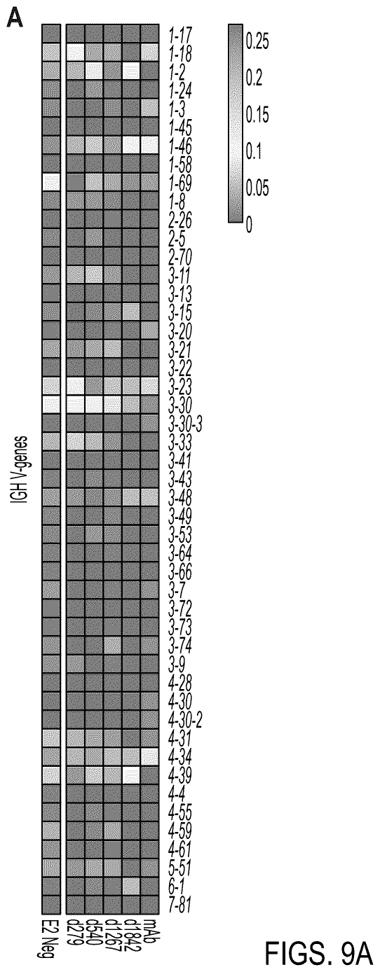

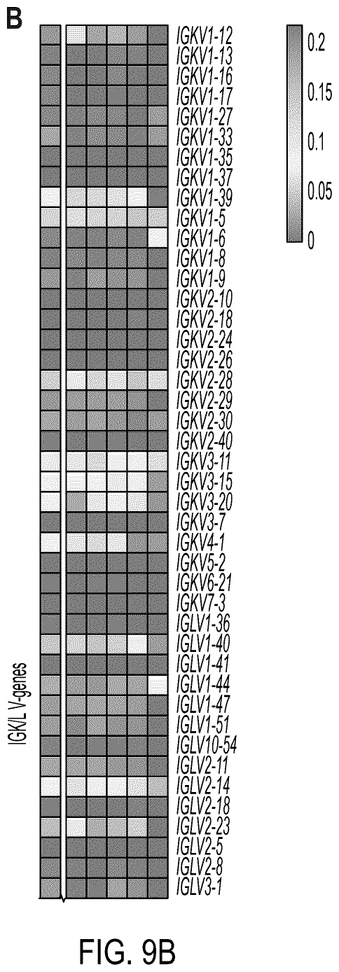

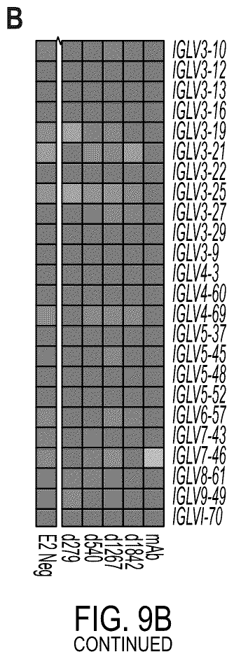

Aa mixture of 3 antigenically diverse subtype 1a and 1b soluble E2 ectodomain proteins (sE2), designated 1b21, 1a157, and 1b09, were used as antigen bait to capture and sort single E2-reactive mature class-switched B cells (CD19+, CD10−, CD3−, IgM−, IgD−, sE2+; FIGS. 2A and 8), as previously described (Ogega et al., 2022). B cells sorted from D2178 were cultured at limiting dilutions from 20 to 1 B cells per well and stimulated with recombinant IL-2, IL-21 and irradiated CD40L-expressing fibroblasts. Culture supernatants were tested for production of human IgG and the presence of IgG that was reactive with each of the three E2 proteins. All wells cultured at 20 B cells per well (5/5), 85% (92/408) of wells with 4 B cells, 58% (188/324) of wells with 2 B cells, and 26% (70/270) of wells with 1 B cell were positive for IgG production (FIG. 2B). Supernatants from 2 B cells per well cultures reacted with 1b21, 1a157, and 1b09 sE2 at frequencies of 42/324 wells (13%), 100/324 wells (31%), and 105/324 wells (32%), respectively. Supernatants from 1 B cell per well cultures reacted with 1b21, 1a157, and 1b09 sE2 at frequencies of 13/270 wells (5%), 58/270 wells (21%), and 34/270 wells (13%), respectively (FIG. 2B). Of the 259 IgG positive, 2 or 1 B cell supernatants, 64% were reactive with only 1 E2 variant, 22% were cross-reactive with 2 variants, and 9% were cross-reactive with all three E2 variants (FIG. 2C). We RT-PCR amplified and sequenced 55 pairs of heavy and light chain variable sequences, from 2 or 1 B cell cultures that were cross-reactive with at least two of three E2 proteins. Sanger sequencing of all PCR products demonstrated single peaks at each nucleotide position, confirming that all cultures were monoclonal. Taken together, utilization of a mixture of three E2 proteins as bait to isolate E2-reactive B cells followed by in vitro stimulation and testing of supernatants for IgG and E2 reactivity, allowed the sensitive and robust selection and sequencing of E2 cross-reactive, authentically paired heavy and light chains.

E2 reactive and nonreactive class-switched, mature B cells were also sorted from PBMC isolated at 279, 540, 1267, and 1842 dpi, and subjected to BCR-seq. E2 nonreactive BCR sequences from all the timepoints were pooled for analysis and E2 reactive sequences were analyzed separately for each timepoint. These sequences, along with sequences of the 55 mAbs, were analyzed for the presence of somatic mutations, CDR3 lengths, and V or D gene usage. Relative to E2 nonreactive B cells, CDRH3 length increased on d540 (p=0.04), d1267 (p=0.007), and d2178 (p=0.04) (FIG. 2D). There was no statistically significant difference in light chain CDR3 lengths (FIG. 2E). In comparisons of somatic mutation frequencies, defined as BCR nucleotide sequence identity to inferred germline V-gene sequences, there were multiple statistically significant differences. Relative to E2 negative BCRs, d1267 (p<0.0001), d1842 (p=0.0006), and d2178 (mAbs; p<0.0001) BCRs displayed increased somatic mutation frequencies (FIG. 2F). Both heavy and light chains also showed a general trend of increasing somatic mutation over time (FIGS. 2F-2G). Of note, among the 55 mAbs, the median (range) V_{H}-gene nucleotide identity to germline was relatively high at 93.4% (87.9-99.0%). Overall, relative to E2 nonreactive BCRs, we observed increased E2-reactive CDRH3 length and increased somatic mutation frequencies in E2-reactive BCR heavy and light chains that increased over time.

### V_{H}1-69 and D2-2/D2-15 Usage Favor Broad Neutralizing Activity but are not Required.

Sequence analysis of the 55 mAb heavy chains revealed 16 different V_{H} gene segments. V_{H}1-69 was the most frequent, found in 12 of the 55 mAbs (22%), followed by V_{H}1-18, which was found in 9 of 55 mAbs (16%).

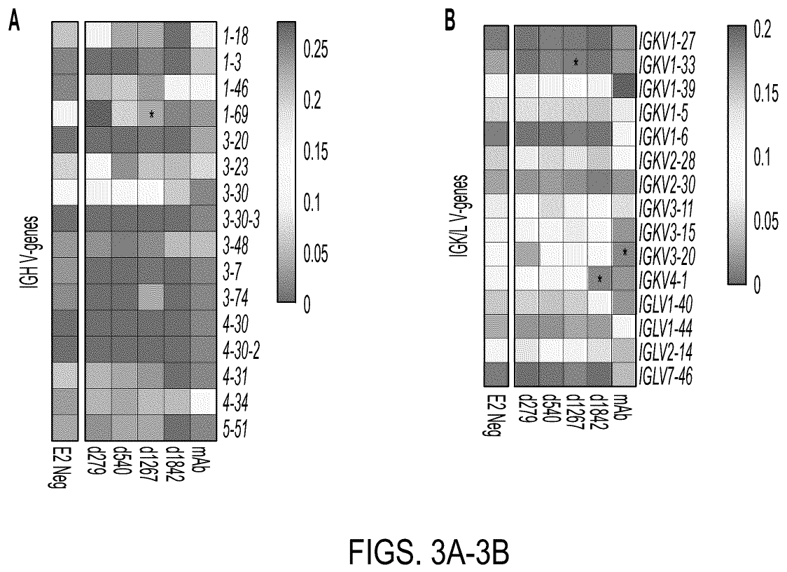

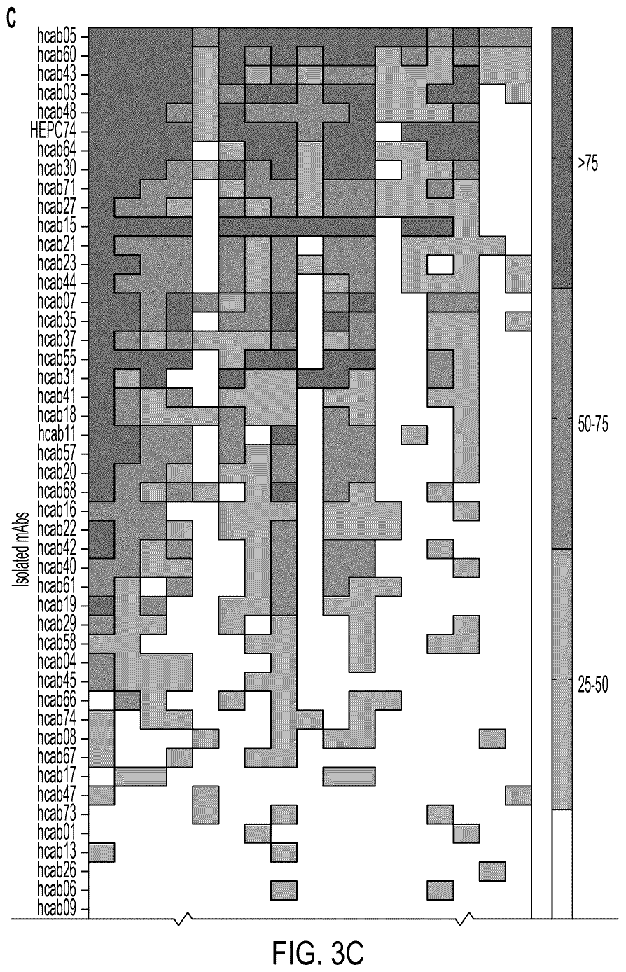

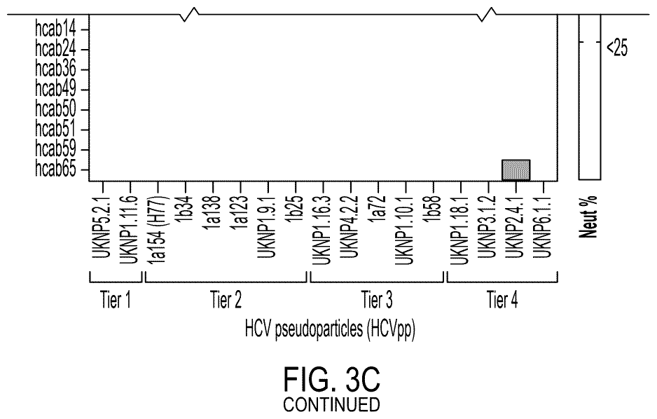

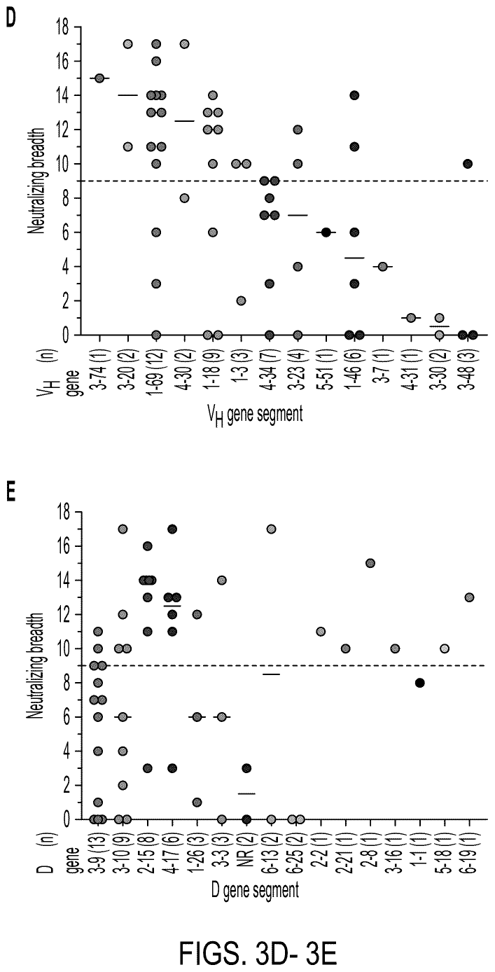

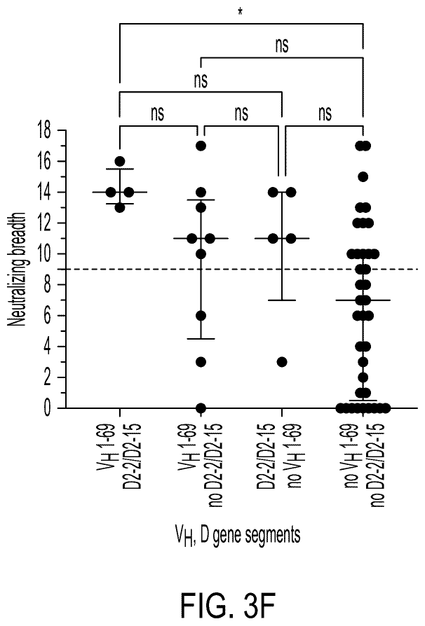

The V-gene usage of longitudinal E2-reactive and nonreactive BCRs was also compared. There was a trend towards higher usage of the V_{H}1-69 gene segment in E2-reactive BCRs from all timepoints relative to E2 nonreactive BCRs, and this difference was statistically significant for d1267 (p=0.0004) (FIG. 3A). Additionally, E2-reactive BCRs utilized V_{K}4-1 more frequently (p=0.001) and V_{K}1-33 less frequently (p=0.001) relative to E2-nonreactive BCRs (FIG. 3B).

Next, immunoglobin variable sequences were synthesized and cloned into human IgG1, Ig Kappa, or Ig Lambda expression plasmids, co-expressing authentically paired heavy and light chains in human embryonic kidney cells to generate IgG1 isotype mAbs, which were designated hepatitis C antibodies (hcabs). Neutralizing breadth of these mAbs was measured using the previously described panel of 17 HCVpp. The majority neutralized tier 1 (i.e., highly sensitive) HCVpp, and 29 mAbs (53%) neutralized 9 or more HCVpps, which is the threshold that we use to define bNAbs (FIG. 3C). These 29 bNAbs utilized a wide range of V_{H} gene segments: V_{H}1-69 (9 mAbs), V_{H}1-18 (5 mAbs), V_{H}1-46, V_{H}1-3, V_{H}3-20, V_{H}3-23, and V_{H}4-34 (2 mAbs each), and V_{H}3-74, V_{H}3-48, V_{H}3-74, and V_{H}4-30 (1 mAb each) (FIG. 3D). The three most broadly reactive bNAbs neutralized 17 of the 17 HCVpps and utilized V_{H}1-69, V_{H}3-20, or V_{H}4-30 gene segments (FIG. 3D).

The inventors previously identified two cysteine residues that form a disulfide bond motif in CDRH3 as another commonly observed feature of HCV bNAbs (Flyak et al., 2018). Two cysteine residues are encoded by members of the IGHD2 gene family, either D2-2 or D2-15. Of the 55 mAbs, 9 (16%) used D2-2 or D2-15 gene segments and 8 of these mAbs were broadly neutralizing (FIG. 3E). However, 21 other bNAbs used a wide range of other D gene segments with the 3 most potent bNAbs (neutralizing all 17 HCVpp) encoded by D3-10, D4-17, or D6-13 (FIG. 3E). It was then investigated whether V_{H}1-69 or D2-2/D2-15 usage predicted broadly neutralizing activity. All 4 mAbs that used both V_{H}1-69 and D2-2/D2-15, 6 of 9 V_{H}1-69 mAbs that did not use D2-2/D2-15, 4 of 5 D2-2/D2-15 mAbs that did not use V_{H}1-69, and 15 of 37 mAbs that did not use V_{H}1-69 or D2-2/D2-15, were bNAbs (FIG. 3F). From these data, it was concluded that of all E2-reactive B cells, approximately 16% expressed bNAbs. E2 reactive mAbs that use V_{H}1-69 and/or D2-2/D2-15 are likely to be bNAbs. However, it was also observed that the majority of HCV bNAbs (15 of 29), including two of the three most potent bNAbs, utilized alternative V_{H} and D gene segments, indicating that broad neutralization does not require V_{H}1-69 or D2-2/D2-15 usage.

HCV bNAbs Target Conserved Front Layer and Novel Epitopes on the E2 Protein.

To determine the epitope specificity of isolated bNAbs, the loss of ELISA binding to sE2 proteins, was first measured with multiple substitutions in the front layer of variant 1a157 E2 (1a157 FRLY KO) or in the antigenic site 412 (AS412) epitope of variant 1a53 E2 (1a53 AS412 KO), proteins designed to abrogate binding of previously described front-layer or AS412-reactive bNAbs while preserving other conformational epitopes (Weber et al., 2022a). Loss of binding affinity assays were also performed using a panel of 25 variant H77 E1E2 single alanine mutant variants selected based upon known critical binding residues of previously described bNAbs targeting diverse epitopes (see Method Details) (Giang et al., 2012b, Kinchen et al., 2018a). In all experiments, % mAb binding to mutant proteins was calculated relative to binding of wild-type 1a157 E2, 1a53 E2, or H77 E1E2 controls. In addition to loss of binding ELISAs, binding competition was measured between each mAb and reference non-neutralizing mAb ARIA that binds the E2 back layer. To aid the interpretation of these results, 11 previously described HCV mAbs with known binding sites were also mapped. These mAbs were HEPC74 and AR3A (E2 front layer; antigenic region 3; (Bailey et al., 2017, Law et al., 2008a)), HC-1 (E2 front layer; Domain B; (Keck et al., 2008)), HC.84.26 (E2 front layer; Domain D; (Keck et al., 2012)), AR4A, AR5A, and HEPC111 (E2 stalk, requiring intact E1E2 heterodimer (Giang et al., 2012a, Keck et al., 2012, Colbert et al., 2019)), HEPC112 (E1), ARIA (E2 back layer; antigenic region 1), HCV1 (AS412; (Broering et al., 2009)), and HEPC108 (E2 front layer-back layer; (Colbert et al., 2019)).

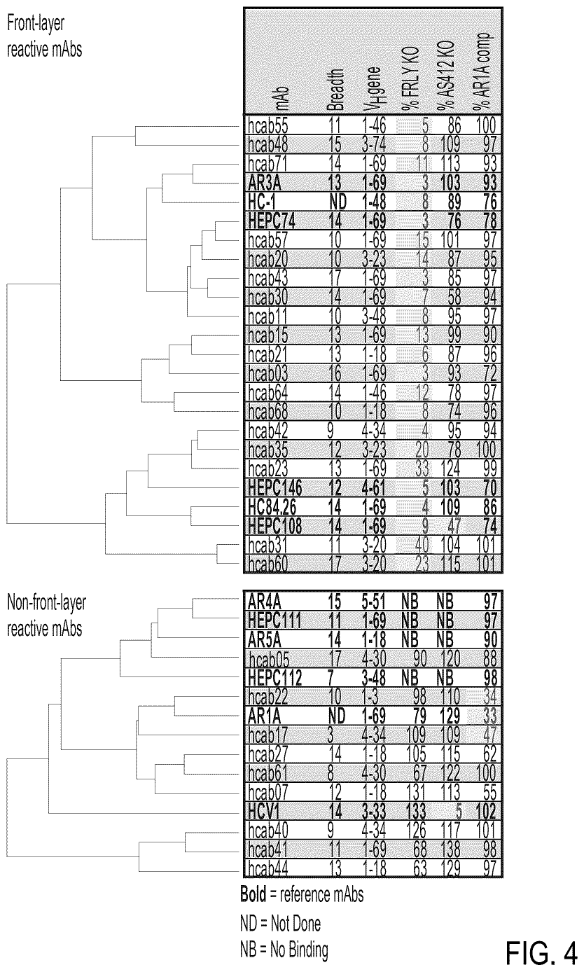

These mAbs were first grouped into front layer-reactive (FRLY) and not front layer-reactive (non-FRLY) groups based on loss of binding to the 1a157 FRLY KO E2 protein. Separately for mAbs in the FRLY and non-FRLY groups, hierarchical clustering analysis was then performed based on loss of binding to the panel of 25 single-alanine mutant E1E2 proteins. Of note, 3 bNAbs that did not bind well to wild-type H77 E1E2 were excluded from this analysis. All reference mAbs clustered as expected in this analysis. Of the 27 novel mAbs that were successfully mapped, 18 (67%) targeted the front layer and 9 (33%) targeted other epitopes on E2 (FIG. 4). The FRLY bNAbs used a wide range of V_{H} gene segments and clustered into 4 distinct groups. One of these groups clustered with HEPC74, AR3A, and HC-1 reference mAbs and another clustered with HEPC146, HC84.26, and HEPC108 reference mAbs. The third (hcab48, 55) and fourth (hcab03, 15, 21, 64, 68) FRLY bNAb groups did not include any reference mAbs (FIG. 4). From these data, it was concluded that the majority of the isolated bNAbs targeted the front layer of E2, with some variation in critical binding residues among FRLY bNAbs.

The non-FRLY mAbs clustered into 5 groups. The first group comprised reference mAbs AR4A, HEPC111, AR5A, HEPC112 and novel bNAb hcab05. AR4A and AR5A target the stalk region of E2 and require intact E1E2 for binding. Similarly, HEPC111 and HEPC112 require the E1E2 heterodimer for binding. Notably, unlike the reference mAbs in this group, hcab05 was able to bind either E1E2 or monomeric E2 in an ELISA. Thus, hcab05 may target an epitope near the stalk of E2, similar to epitopes of AR4A and AR5A. However, unlike these bNAbs, it does not require E1 for E2 stabilization. Notably, hcab05 was also more broadly neutralizing than any of the reference mAbs. The second non-FRLY group was composed of hcab22, ARIA, and hcab 17. Hcabs 22 and 17 also competed for E1E2 binding with ARIA, indicating that they target the back layer of the E2 protein. Like ARIA, a weakly neutralizing antibody, hcab17 also displayed poor neutralizing activity. Unexpectedly, hcab22 was a bNAb despite clustering together with ARIA (FIG. 4). The third group of non-FRLY mAbs was composed of hcab27, hcab61, and hcab07. These bNAbs did not cluster with any reference mAbs, compete with ARIA, or bind AS412 and thus also target a novel bNAb epitope on the E2 protein. Reference mAb HCV1 clustered alone in group 4, demonstrating that none of the novel bNAbs target AS412, which is consistent with prior reports showing that this epitope is rarely targeted in human infection (Tarr et al., 2006, Kinchen et al., 2019). Group 5 was composed of bNAbs hcab40, hcab41, and hcab44, targeting yet another novel non-FRLY neutralizing epitope on the E2 protein (FIG. 4). Taken together, non-FRLY bNAbs targeting multiple novel epitopes were identified. Hcab05 may target an epitope near the stalk of E2 but is unique among bNAbs in that class as it is not dependent on E1 for binding. Hcab22 is broadly neutralizing despite binding to antigenic region 1 in the back layer of E2, which is typically a non-neutralizing antigenic site.

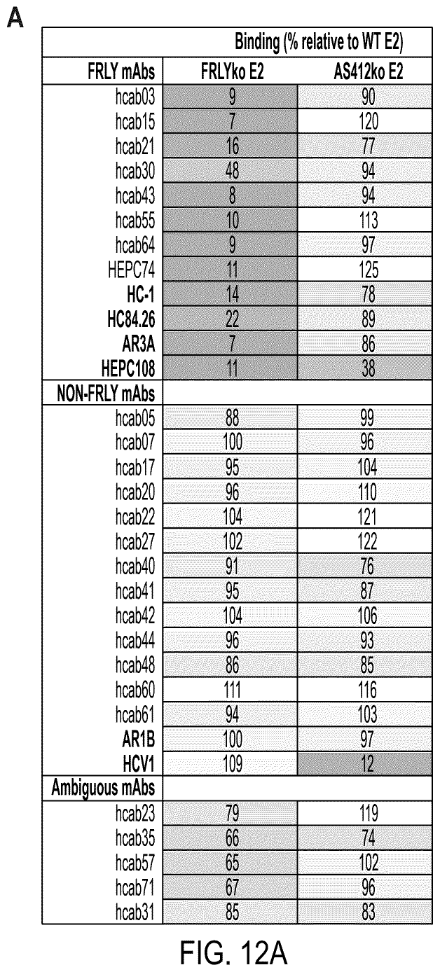

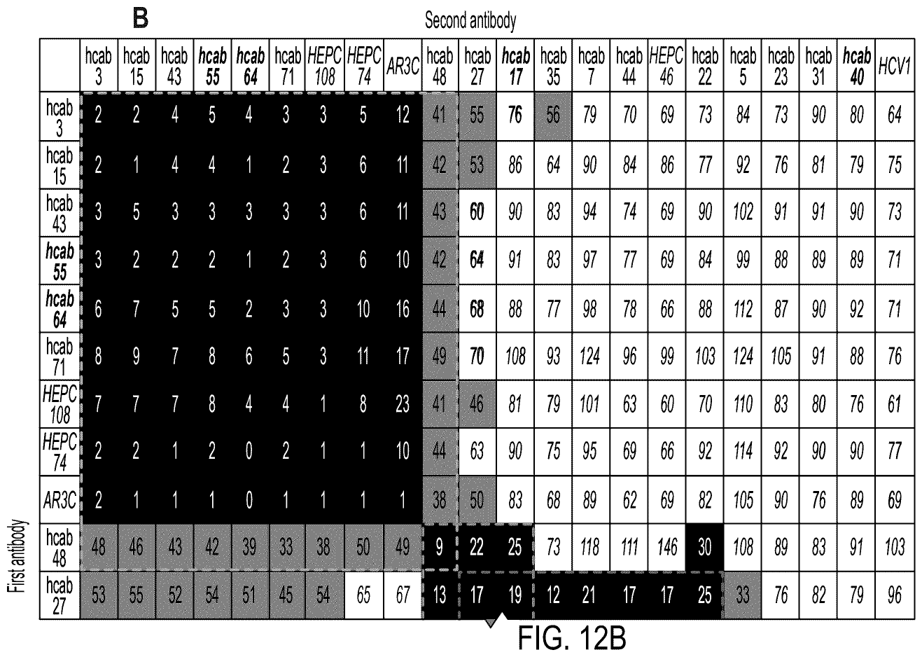

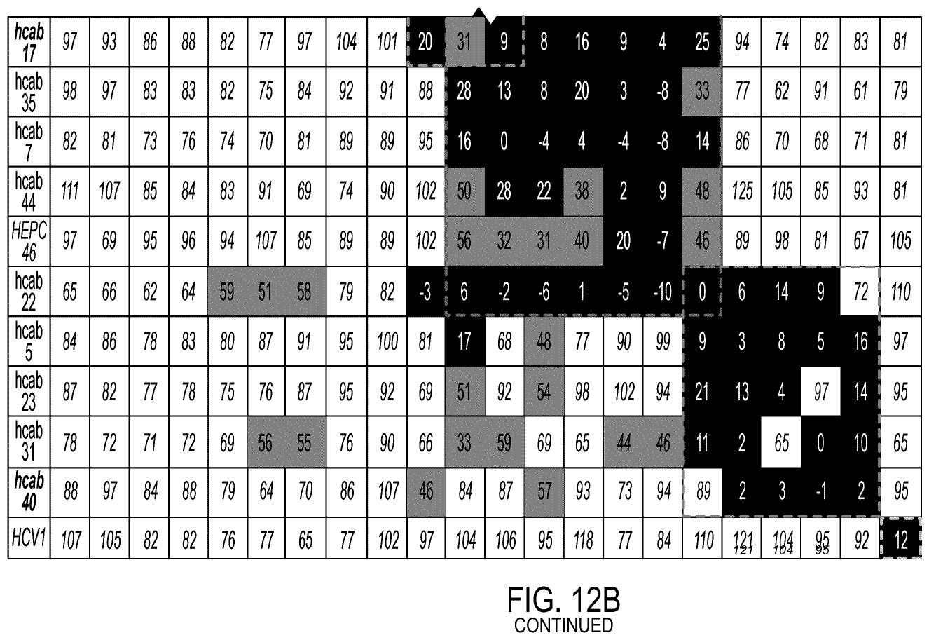

To determine which antigenic regions of the E2 glycoproteinare targeted by the non-FRLY mAbs and ambiguous mAbs, we performed E2-binding competition experiments using BioLayer interferometry (FIG. 12B). We also included a panel of reference antibodies with known epitope specificity in this analysis. These mAbs were HEPC74, HEPC108, AR3C (E2 FRLY; antigenic region 3), HEPC46 (E2 b sandwich; antigenic region 1), and HCV1 (AS41235). Hcabs 41, 42, 57, 60, and 61 did not bind to biotinylated 1a157 E2 in this competition assay and were excluded from the analysis. Consistent with the FRLY KO binding ELISAs, six bNAbs (hcab3, hcab15, hcab43, hcab55, hcab64, and hcab71) competed for binding with each other and with FRLY reference mAbs. In the non-FRLY group, four mAbs (hcabs 7, 22, 35, and 44) competed with previously structurally characterized non-neutralizing mAb HEPC46, which targets the β sandwich antigenic region, and three mAbs (hcabs 48, 27, and 17) competed for binding to a FRLY/b sandwich overlapping antigenic region. The remaining five non-FRLY bNAbs (hcabs 5, 22, 23, 31, and 40) bound to a region of E2 distinct from the β sandwich or FRLY antigenic regions.

Structural Analysis Reveals Conserved bNAb-E2 Interactions.

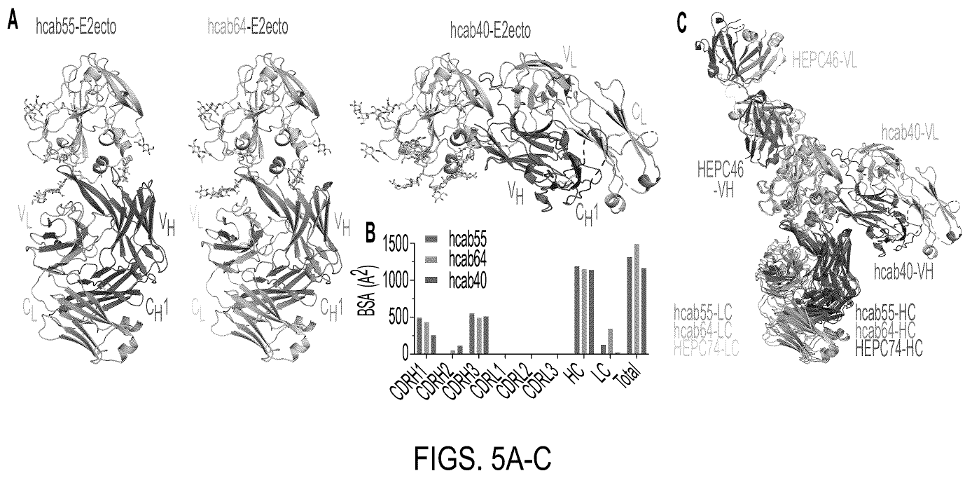

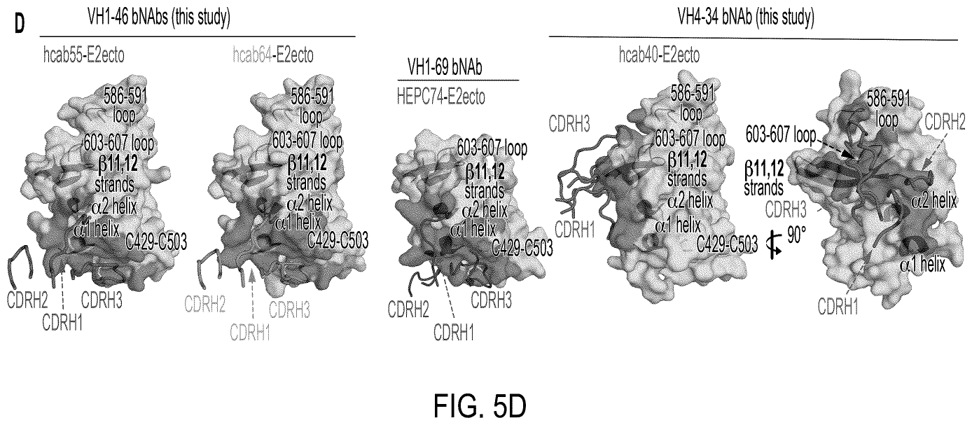

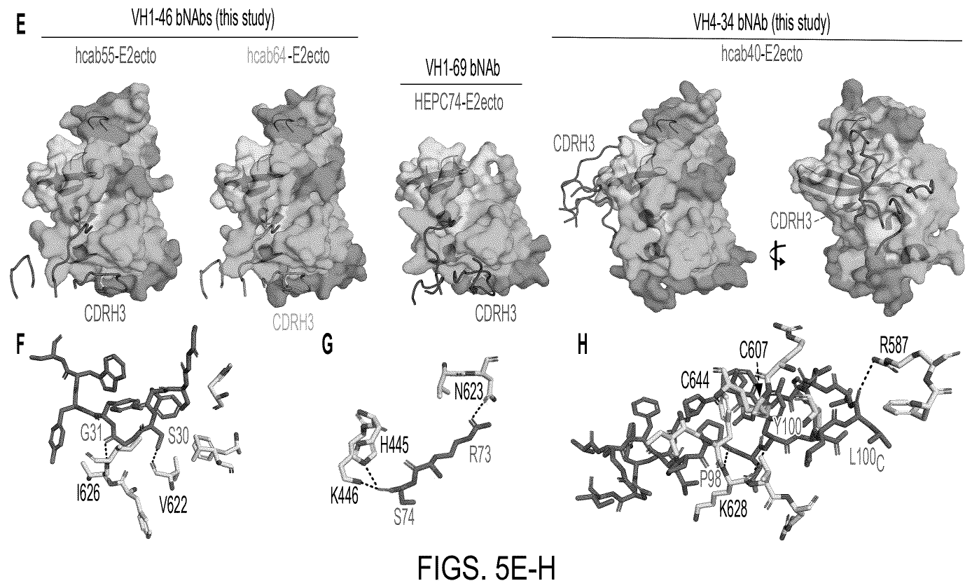

To verify the epitope specificity of non-V_{H}1-69 bNAbs, crystal structures of hcab55 and hcab64 (FRLY bNAbs) were determined, as well as hcab40 (a non-FRLY bNAb) in complex with E2 ectodomains (E2ecto). Crystal structures of E2 complexed with hcab55 and hcab64 (two V_{H}1-46-encoded bNAbs) as well as hcab40 (a V_{H}4-34-encoded bNAb) are the first X-ray structures of non-V_{H}1-69 bNAb-E2 complexes (FIG. 5A).

The structures of the two V_{H}1-46 bNAbs, hcab55 and hcab64, were remarkably similar to the previously described crystal structure of V_{H}1-69 bNAb HEPC74, which was also isolated from subject C110. hcab55, hcab64, and HEPC74 share 22 interface residues that are almost exclusively located in the FRLY of E2; many of these residues are highly conserved across HCV genotypes (Table 2). As found for other front-layer-reactive bNAbs, both V_{H}1-46 bNAbs contact E2 primarily by V_{H} residues, burying 1,188 or 1,148 Å² (91% or 77% of the total Fab buried surface area, BSA) for hcab55 and hcab64, respectively (FIG. 5B). While the majority of hcab55 and hcab64 contacts with E2 involve CDRH1 and CDRH3, CDRH2, which can play an important role in the recognition of hydrophobic residues in the E2 front layer (Flyak et al., 2018; Kong et al., 2013; Tzarum et al., 2019), plays a minimal role among the two V_{H}1-46 bNAbs structurally characterized in this study.

Although they are encoded by different V_{H}-genes, hcab55 and hcab64 (V_{H}1-46 bNAbs) and HEPC74 (V_{H}1-69 bNAb) utilize similar approach angles (FIG. 5C) to recognize the overlapping epitopes in the E2 FRLY (FIG. 5D). A similar approach angle of three bNAbs (FIG. 5E) is likely a result of the straight conformation of disulfide bridge-containing CDRH3 loops of bNAbs that interact with shared E2 contact residues in the FRLY and CD81 binding loop (FIG. 5B). This similar binding orientation between HEPC74 and two V_{H}1-46 bNAbs places the CDRH1 loop of these bNAbs in a similar position near the N terminus of the E2 al helix (FIG. 5D) and CDRH3 loops near the C429 residue in the front layer (FIG. 5H). E2 residues hydrogen bonded by hcab55, hcab64, and HEPC74 included C429 and N448 (bound by shared D- or V_{H}-gene germline residues C100 or T28, respectively), K446 (bound by shared somatic substitution I30), D431 (bound by junctional residues A/G/K98), and T435 (bound by either germline residue Y32 in HEPC74 or T28 in hcab55 and hcab64). The key interacting residues identified by the structural analysis were consistent with mapping data obtained using E2 domain knockouts, alanine scanning mutagenesis, and ARIA binding competition assays (FIG. 4).

Finally, the crystal structure of V_{H}4-34 bNAb hcab40 in complex with E2ecto revealed a novel E2 neutralizing epitope in the back layer of E2 (FIGS. 5A, 5C-5E). Previously, the inventors isolated a weakly neutralizing mAb HEPC46, which recognized the E2 central β sandwich (FIG. 5C) (Flyak et al., 2018). The weak neutralizing activity of HEPC46 was likely explained by the proximal location of the HEPC46 epitope in the E2β sandwich, where HEPC46 is unlikely to prevent E2 from binding to the CD81 receptor. Similarly to mAbs that recognize the E2β sandwich, back layer-reactive mAbs are thought to be non-neutralizing (Hadlock et al., 2000). Surprisingly, hcab40 neutralizes Tier 1-3 HCVpp (FIG. 3C), predominantly recognizing residues in the back layer of E2. Back layer residues bury 785 Å² (66% of the total E2 BSA) in the hcab40-E2ecto structure (FIGS. 5D and 5E, Table 3). While hcab40 CDRH1 and CDRH3 residues form several putative hydrogen bonds with residues in the back layer of E2 (FIGS. 5F and 5H), the S74 residue in the FR3 region of hcab40 also makes two hydrogen bonds with the E2 front layer (FIG. 5G). Notably, the two E2 FRLY residues contacted by hcab40, H445 and K446, are also interface residues for FRLY bNAbs HEPC74, hcab55, and hcab64. The existence of a novel neutralizing epitope in the back layer of E2 targeted by a non-V_{H}1-69 bNAb suggests the possibility of a synergistic antibody response between FRLY-reactive V_{H}1-69 or V_{H}1-46 bNAbs (HEPC74, hcab55, and hcab64) and a V_{H}4-34 back layer-reactive bNAb (hcab40) (FIG. 5B).

Taken together, epitope mapping and structural analyses revealed that bNAbs use diverse V_{H} and D-genes to target conserved front layer and novel epitopes in the back layer or stalk region of E2. Although FRLY bNAbs target a variety of slightly different epitopes within the front layer based upon E2 alanine scanning analysis, structures revealed remarkable conservation of approach angles and epitopes of FRLY bNAbs encoded by either V_{H}1-46 or V_{H}1-69 gene segments.

HCV bNAbs Appear Early in Infection and Display Convergent Evolution.

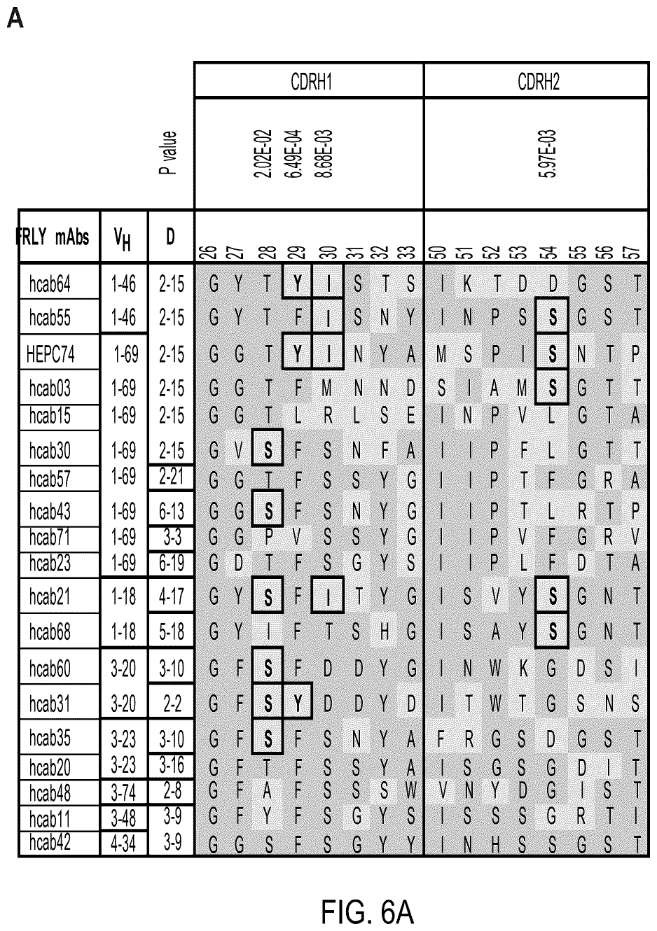

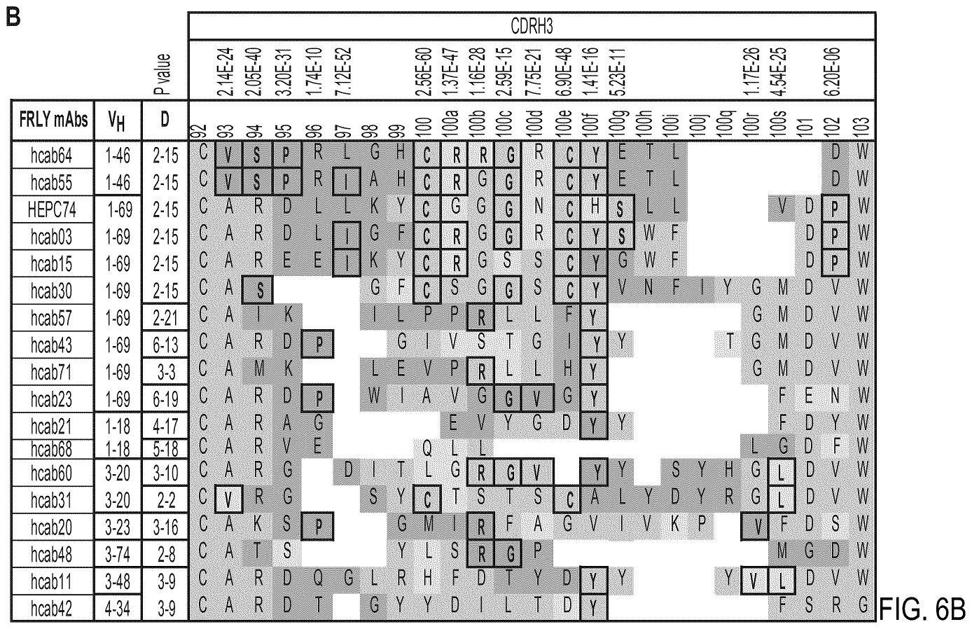

Given remarkable similarity among binding epitopes of FRLY bNAbs utilizing diverse V_{H} and D-genes, we hypothesized that these mAbs may have evolved convergently. The bNAb sequences were analyzed for shared residues arising in CDRH1-2 by somatic mutation (i.e. non-germline residues), and emergence of the same substitutions in multiple bNAbs regardless of V- or D-gene usage was discovered (FIG. 6A). To determine whether each shared CDRH1-2 substitution was significantly enriched in FRLY bNAbs, the proportion of FRLY bNAbs with a given substitution were compared to the proportion of E2 nonreactive BCRs with that same substitution. E2 nonreactive BCRs included in this analysis were isolated from the same study subject and matched to the FRLY bNAbs by V_{H}-gene usage and germline homology. S28, Y29, and I30 (in CDRH1) and S54 (in CDRH2) substitutions were each significantly enriched in FRLY bNAbs (p=0.02, 0.0006, 0.009, or 0.006, respectively; FIG. 6A). S28 arose in bNAbs utilizing V_{H}1-69, 1-18, 3-20, or 3-23. Y29 arose in bNAbs utilizing V_{H}1-46, 1-69, or 3-20. Notably, Y29 formed a main-chain hydrogen bond with E2 residue T435 in the hcab64-E2 crystal structure and was also an E2-interface residue in the HEPC74-E2 crystal structure. 130 arose in bNAbs utilizing V_{H}1-46, 1-69, or 1-18, and formed a main chain hydrogen bond with E2 residue K446 in the hcab55-E2, hcab64-E2, and HEPC74-E2 crystal structures. S54 arose in bNAbs utilizing V_{H}1-46, 1-69, or 1-18 and was an interface residue in the HEPC74-E2 crystal structure.

To identify significantly enriched amino acids within CDRH3 of FRLY bNAbs, FRLY bNAb CDRH3 sequences were aligned with sequences of E2 nonreactive CDRH3s from the same subject (matched based on CDRH3 lengths) (FIG. 6B). 16 CDRH3 amino acid residues were identified that were significantly enriched in FRLY bNAbs. These residues were either germline encoded or encoded by somatic mutations or junctional insertions. Notably, an R100a CDRH3 substitution arose in both V_{H}1-46 and V_{H}1-69-encoded bNAbs using D2-15 (p=1E-47). In the hcab55-E2 crystal structure, R100a formed two hydrogen bonds and a salt bridge with E531 of E2 and was also an interface residue in the hcab64-E2 crystal structure. An R100b CDRH3 substitution (p=1E-28) arose by somatic mutation in one bNAb using D2-15, by junctional insertion in bNAbs using D2-21, D3-16, or D2-8, and was germline encoded in bNAbs using D3-3 or D3-10. In the hcab64-E2 crystal structure, R100b formed three salt bridges and a hydrogen bond with E531 in E2. Finally, it was noted that the majority of FRLY bNAbs (14/19) had a Y at position 100f in CDRH3 (p=1E-16). Although this was not an E2 interface residue in E2-bNAb structures, it was noteworthy because of its high frequency and because it was germline encoded in some bNAbs and arose by junctional insertion in others. Taken together, this analysis showed that some amino acids were present in FRLY bNAbs significantly more than would be expected by chance, and many of these residues fell at the interface with E2.

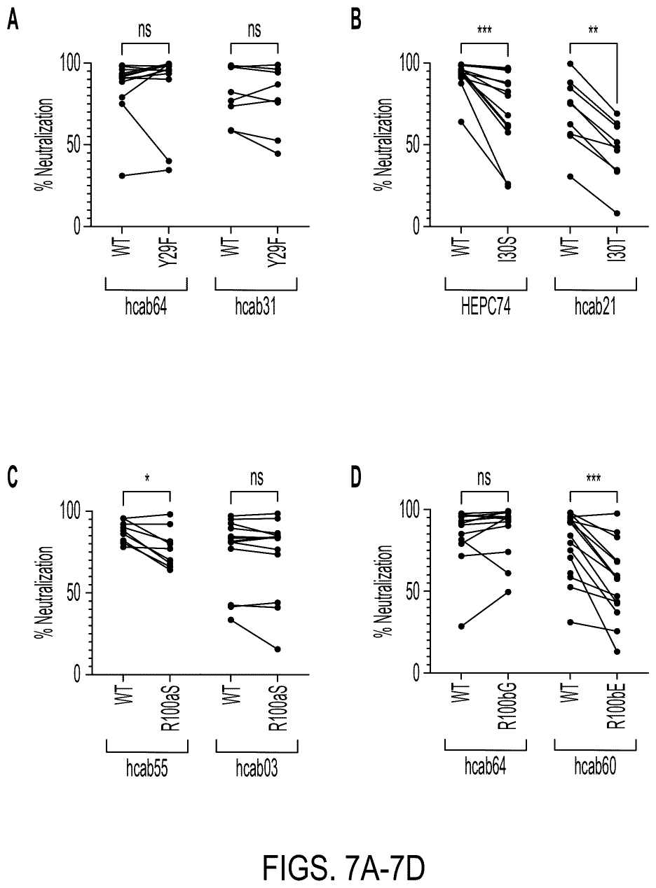

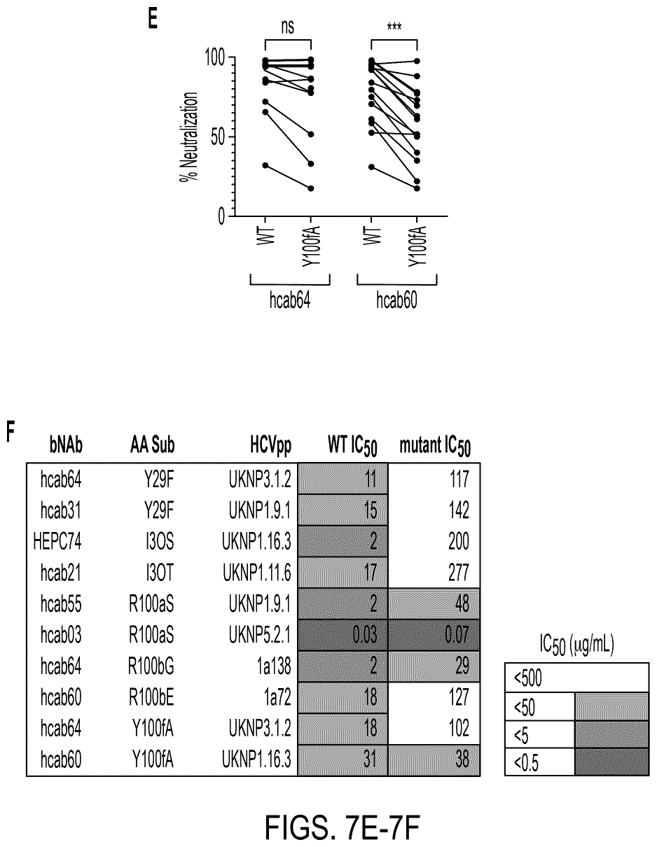

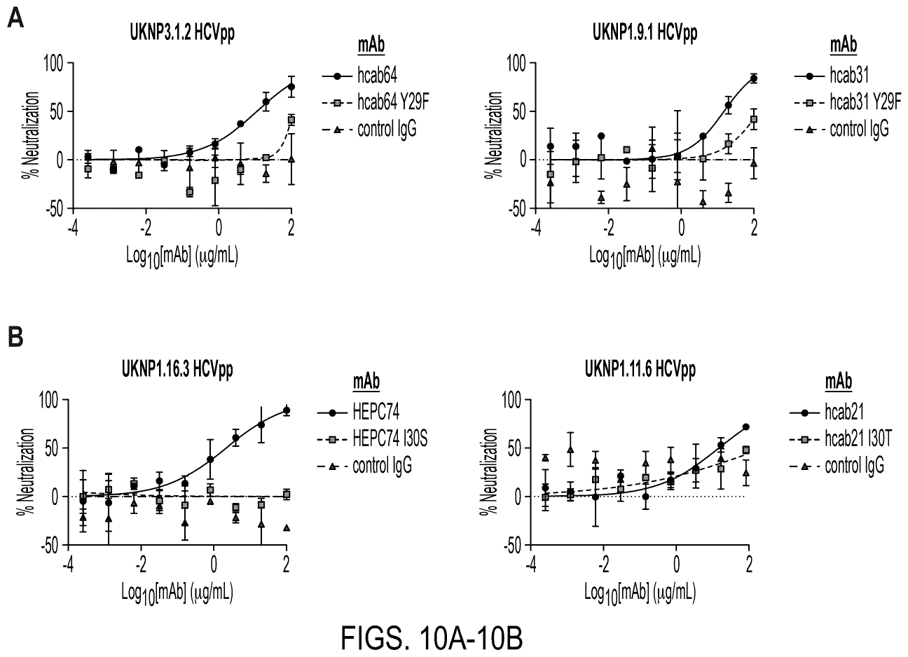

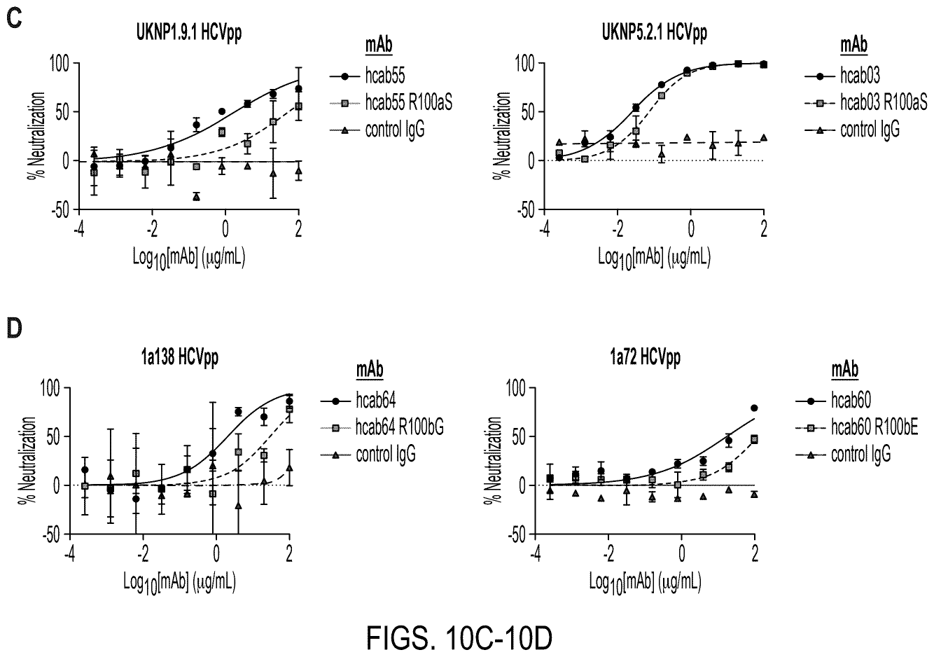

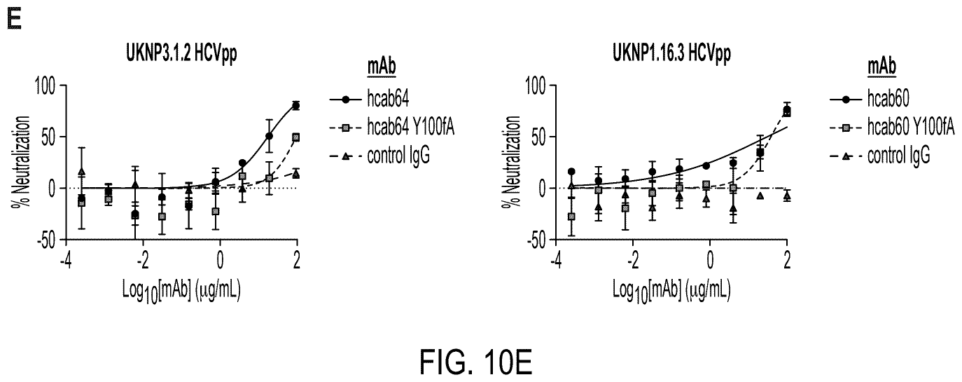

To confirm the functional significance of these enriched, shared residues, a subset was selected for mutagenesis analysis. Site-directed mutagenesis was used to individually revert somatic mutation-encoded Y29, 130, or R100a substitutions in two bNAbs with two different V-genes to the germline-encoded amino acid (F, T/S, or S, respectively) (FIG. 7A-C). Somatic mutation-encoded R100b was also reverted to the germline-encoded glycine residue in V_{H}1-46 bNAb hcab64, and germline-encoded R100b to glutamic acid residue in V_{H}3-20 bNAb hcab60 (FIG. 7D). Finally, in hcab64, where Y100f was the germline-encoded residue, and in hcab60, where Y100f arose by junctional insertion, this residue was mutated to alanine (FIG. 7E). The relative neutralizing breadth of wild-type and mutant bNAbs was measured across the panel of 17 HCVpp. Reversions at each position except Y29 led to significantly reduced neutralizing activity across the HCVpp panel by one or more hcabs, confirming that these substitutions were important for neutralizing breadth. Next, neutralization of a single HCVpp was tested by serial dilutions of each WT and mutant hcab pair (FIGS. 7F and 10A-10E). Each reversion led to loss of neutralizing potency by both hcabs that were tested. Notably, although the Y29F did not have a statistically significant effect across the full HCVpp panel, this reversion caused hcab64 and hcab31 to each lose potency against some heterologous HCVpp, indicating that this substitution enhances neutralizing breadth in an HCV variant-specific manner. Taken together, these site-directed mutagenesis experiments confirmed that substitutions in CDRH1 and 3 that were enriched and shared across diverse bNAbs enhanced the neutralizing breadth and potency of those bNAbs.

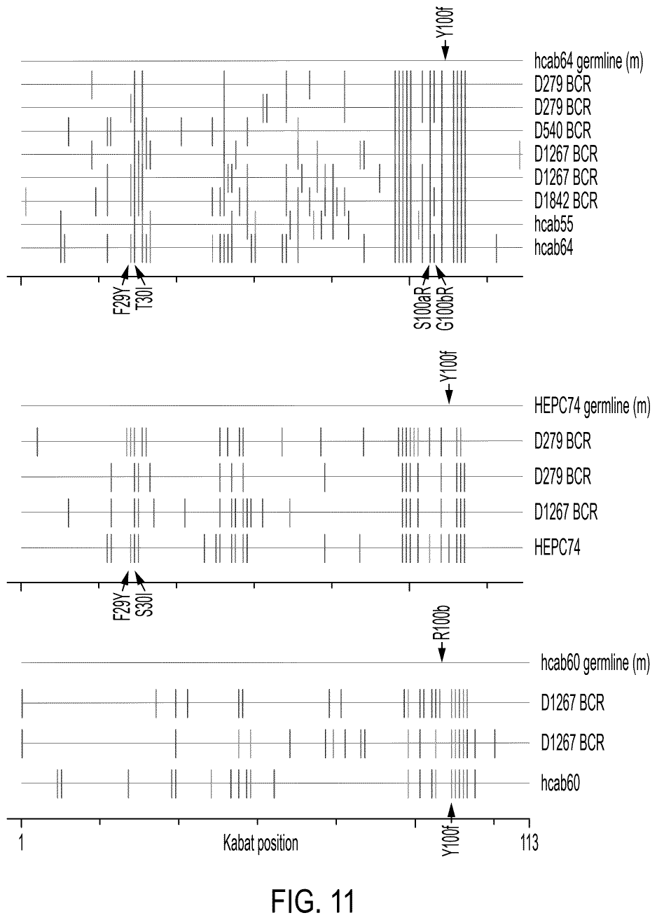

For many FRLY bNAbs, BCRs were identified from the same B cell lineage in the longitudinal BCR-seq dataset obtained at days 279, 540, 1267, and 1842 post infection (FIG. 11), based upon matched V and J-gene usage and identical CDRH3 length. These longitudinal BCR lineage sequences were used to define the evolution of shared bNAb CDRH1-3 substitutions over time. Notably, 130 substitutions in CDRH1 appeared in multiple bNAb lineages early in infection at D279 and were found in all BCR sequences at every timepoint of the hcab55/64 and HEPC74 lineages. Other bNAb-enriched amino acids, including S100aR and G100aR, were also detected as early as D279 after infection and maintained in each lineage until D2178 when mAbs were isolated, although many substitutions were not present universally in all members of each bNAb lineage. For example, F29Y was detected in the hcab55/64 bNAb lineage at D279, D1267, D1842, and D2178, but was present in only 4 of 8 sequences in that lineage (FIG. 11), which might be explained by the observation that the phenotype of this substitution is HCV variant-specific. Overall, longitudinal BCR-seq analysis revealed that multiple FRLY bNAb B cell lineages were already detectable early after infection, and many substitutions that were shared across multiple bNAbs also arose early in infection and were then maintained for the entire ~1900 day sampling period. Early appearance and maintenance over time of these shared substitutions in a person who cleared infection repeatedly supports their importance for neutralizing breadth.

TABLE 1
X-ray data collection and refinement statistics

| | | | |
|---|---|---|---|
| | hcab55 Fab | hcab64 Fab | hcab40 Fab |
| PDB ID | E2ecto [symbol] | E2ecto [symbol] | E2ecto [symbol] |

Data collection^{a, b}

| | | | |
|---|---|---|---|
| Space group | P2 [symbol] 2 [symbol] 2 [symbol] | P2 [symbol] 2 [symbol] 2 [symbol] | P3 [symbol] 21 |
| Cell Dimenstions | | | |
| a, b, c (Å) | 67.0, [symbol] , | 68.7, 88.2, | 79.9, 79.9, |
| | 175.4 | 174.7 | 294.6 |
| α, β, γ (°) | 90, 90, 90 | 90, 90, 90 | 90, 90, 120 |
| Resolution (Å) | 43.9-2.6 | 43.7-2.1 | 40.0-3.1 |
| | (2.7-2.6) | (2.2-2.1) | (3.2-3.1) |
| R_{merge} (%) | 16.5 | 16.3 | 16.7 |
| | (124.2) | (262.0) | ( [symbol] ) |
| R [symbol] (%) | 7.5 | 7.1 | 3.8 |
| | ( [symbol] ) | (113.0) | (52.6) |
| CC_{1/2} (%) | 98.3 | 99.6 | 99.9 |
| | (53.2) | (37.6) | (77.0) |
| <I/ [symbol] I> | 6.8 | 7.2 | 11.1 |
| | (1.6) | (1.2) | (1.6) |
| Completeness (%) | 98.6 | 98.8 | 99.7 |
| | (99.2) | (98.6) | (99.7) |
| Redundancy | 5.6 | 6.1 | 19.5 |
| | (5.7) | (6.2) | (20.0) |
| Wilson B-factor | 51.4 | 40.4 | 111.3 |
| (Å²) | | | |

Refinement and Validation

| | | | |
|---|---|---|---|
| Resolution (Å) | 43.9-2.6 | 43.7-2.1 | 40.0-3.1 |
| | (2.7-2.6) | (2.2-2.1) | (3.2-3.1) |
| Unique Reflections | 32,937 | 59,572 | 20,314 |
| | (3,253) | (5,925) | (1,989) |
| Number of atoms | | | |
| Pro [symbol] | 5,033 | 5,055 | 4,928 |
| Ligand | 170 | 173 | 356 |
| R [symbol] /R [symbol] (%) | 20.2/25.8 | 19.8/22.7 | 22.6/27.1 |
| R.m.s. deviations | | | |
| Bond lengths (Å) | 0.009 | 0.01 | 0.009 |
| Bond angles (°) | 1.1 | 1.1 | 1.3 |
| Poor rotamers (%) | 1.6 | 1.6 | 0.4 |
| Ramachandran plot | | | |
| Favored (%) | 95.5 | 96.9 | 94.8 |
| Allowed (%) | 3.9 | 2.6 | 4.9 |
| Disallowed (%) | [symbol] | 0.5 | 0.3 |
| Average B-factor | 57.4 | [symbol] | 133.7 |
| (Å²) | | | |

[symbol]

For each [symbol] , data were derived from a single crystal. [symbol]
Numbers in parentheses correspond to the highest resolution shell. [symbol]
indicates data missing or illegible when filed

Data collection and refinement statistics for hcab55 Fab-1b09 E2ecto, hcab64 Fab-1b09 E2ecto, and hcab40 Fab-1b09 E2ecto complexes.

TABLE 2
Interface residues between hcab55/hcab64
Fabs and E2ecto proteins.

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | [symbol] | [symbol] | [symbol] | [symbol] | | [symbol] |
| [symbol] | [symbol] | [symbol] | [symbol] | [symbol] | [symbol] | [symbol] |
| [symbol] | [symbol] | [symbol] | [symbol] | [symbol] | | [symbol] |
| [symbol] | [symbol] | [symbol] | [symbol] | [symbol] | [symbol] | [symbol] |
| [symbol] | | [symbol] | [symbol] | [symbol] | [symbol] | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | [symbol] | [symbol] | [symbol] | [symbol] | [symbol] | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| [symbol] | | [symbol] | [symbol] | [symbol] | [symbol] | [symbol] |
| | | | [symbol] | [symbol] | | [symbol] |
| | | | [symbol] | [symbol] | | [symbol] |
| | | | [symbol] | [symbol] | [symbol] | [symbol] |
| | | | [symbol] | [symbol] | | [symbol] |
| | | | [symbol] | [symbol] | | [symbol] |
| | | | [symbol] | [symbol] | | [symbol] |
| | | | [symbol] | [symbol] | | [symbol] |
| [symbol] | | [symbol] | | | | [symbol] |

[symbol]

| [symbol] | [symbol] | [symbol] | [symbol] | [symbol] |
|---|---|---|---|---|
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |

[symbol]

| [symbol] | [symbol] | [symbol] | [symbol] | [symbol] |
|---|---|---|---|---|
| [symbol] | [symbol] | [symbol] | [symbol] | [symbol] |
| [symbol] | [symbol] | [symbol] | [symbol] | [symbol] |

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | | [symbol] |
| [symbol] | | [symbol] | [symbol] | | | [symbol] |

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | [symbol] | [symbol] |
| [symbol] | | [symbol] | | | | |
| [symbol] | [symbol] | [symbol] | | | | |
| [symbol] | [symbol] | [symbol] | | | | |
| [symbol] | [symbol] | [symbol] | | | | |
| [symbol] | | [symbol] | | | | |
| [symbol] | | [symbol] | | | | |
| [symbol] | | [symbol] | [symbol] | [symbol] | [symbol] | [symbol] |
| [symbol] | | [symbol] | | | | |
| [symbol] | [symbol] | [symbol] | | | | |
| [symbol] | | [symbol] | | | | |
| [symbol] | | [symbol] | [symbol] | [symbol] | [symbol] | [symbol] |
| [symbol] | | [symbol] | | | | |
| [symbol] | [symbol] | [symbol] | | | | |
| [symbol] | | [symbol] | [symbol] | [symbol] | | [symbol] |
| [symbol] | | [symbol] | [symbol] | [symbol] | | [symbol] |
| [symbol] | | [symbol] | | | | |
| [symbol] | | [symbol] | | | | |
| [symbol] | | [symbol] | | | | |
| [symbol] | | [symbol] | [symbol] | [symbol] | | [symbol] |
| [symbol] | | [symbol] | [symbol] | | | [symbol] |
| [symbol] | | | | | | |
| [symbol] | | | | | | |
| [symbol] | [symbol] | [symbol] | [symbol] | [symbol] | [symbol] | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | [symbol] | [symbol] | [symbol] | [symbol] | | [symbol] |
| [symbol] | [symbol] | [symbol] | [symbol] | [symbol] | [symbol] | [symbol] |
| [symbol] | [symbol] | [symbol] | [symbol] | [symbol] | | [symbol] |
| [symbol] | [symbol] | [symbol] | [symbol] | [symbol] | [symbol] | [symbol] |
| [symbol] | | [symbol] | [symbol] | [symbol] | [symbol] | [symbol] |
| [symbol] | [symbol] | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| | | | [symbol] | [symbol] | [symbol] | [symbol] |
| | | | [symbol] | [symbol] | | [symbol] |
| | | | [symbol] | [symbol] | | [symbol] |
| | | | [symbol] | [symbol] | [symbol] | [symbol] |
| | | | [symbol] | [symbol] | | [symbol] |
| | | | [symbol] | [symbol] | | [symbol] |
| | | | [symbol] | [symbol] | | [symbol] |
| [symbol] | | [symbol] | | | | [symbol] |

[symbol]

| [symbol] | [symbol] | [symbol] | [symbol] | [symbol] |
|---|---|---|---|---|
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |

[symbol]

| [symbol] | [symbol] | [symbol] | [symbol] | [symbol] |
|---|---|---|---|---|
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | | [symbol] |
| | | | [symbol] | [symbol] | | [symbol] |
| | | | [symbol] | [symbol] | | [symbol] |
| [symbol] | | [symbol] | [symbol] | | | [symbol] |

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | [symbol] | [symbol] |
| [symbol] | | [symbol] | | | | |
| [symbol] | [symbol] | [symbol] | | | | |
| [symbol] | [symbol] | [symbol] | | | | |
| [symbol] | | [symbol] | | | | |
| [symbol] | | [symbol] | | | | |
| [symbol] | [symbol] | [symbol] | [symbol] | [symbol] | [symbol] | [symbol] |
| [symbol] | | [symbol] | | | | |
| [symbol] | | [symbol] | | | | |
| [symbol] | [symbol] | [symbol] | | | | |
| [symbol] | | [symbol] | | | | |
| [symbol] | [symbol] | [symbol] | | | | |
| [symbol] | | [symbol] | [symbol] | [symbol] | [symbol] | [symbol] |
| [symbol] | | [symbol] | | | | |
| [symbol] | [symbol] | [symbol] | | | | |
| [symbol] | | [symbol] | | | | |
| [symbol] | | [symbol] | | | | |
| [symbol] | [symbol] | [symbol] | [symbol] | [symbol] | [symbol] | [symbol] |
| [symbol] | | [symbol] | | | | |
| [symbol] | | [symbol] | | | | |
| [symbol] | [symbol] | | | | | |
| [symbol] | | | | | | |
| [symbol] | | | [symbol] | [symbol] | [symbol] | [symbol] |
| [symbol] | | | | | | |
| [symbol] | | | [symbol] | [symbol] | [symbol] | [symbol] |
| [symbol] | | | [symbol] | [symbol] | [symbol] | [symbol] |
| [symbol] | | | [symbol] | [symbol] | | [symbol] |

[symbol]

[symbol]

indicates data missing or illegible when filed

TABLE 3
Interface residues between hcab40 Fab and E2ecto protein.

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | [symbol] | [symbol] |
| [symbol] | | [symbol] | [symbol] | [symbol] | | [symbol] |
| [symbol] | [symbol] | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | | [symbol] |
| [symbol] | [symbol] | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | [symbol] | [symbol] |
| [symbol] | [symbol] | [symbol] | [symbol] | [symbol] | [symbol] | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | | [symbol] |
| | | | [symbol] | [symbol] | | [symbol] |
| | | | [symbol] | [symbol] | | [symbol] |
| | | | [symbol] | [symbol] | | [symbol] |
| [symbol] | | [symbol] | | | | [symbol] |

[symbol]

| [symbol] | [symbol] | [symbol] | [symbol] | [symbol] |
|---|---|---|---|---|
| 1 | [symbol] | [symbol] | [symbol] | [symbol] |
| 2 | [symbol] | [symbol] | [symbol] | [symbol] |
| 3 | [symbol] | [symbol] | [symbol] | [symbol] |
| 4 | [symbol] | [symbol] | [symbol] | [symbol] |
| 5 | [symbol] | [symbol] | [symbol] | [symbol] |
| 6 | [symbol] | [symbol] | [symbol] | [symbol] |
| 7 | [symbol] | [symbol] | [symbol] | [symbol] |
| 8 | [symbol] | [symbol] | [symbol] | [symbol] |

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | | [symbol] |
| [symbol] | | [symbol] | | | | |
| [symbol] | | [symbol] | | | | |
| Total | | [symbol] | Total | | | [symbol] |

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | [symbol] | [symbol] |
| [symbol] | [symbol] | [symbol] | | | | |
| [symbol] | | [symbol] | | | | |
| [symbol] | | [symbol] | | | | |
| [symbol] | [symbol] | [symbol] | [symbol] | [symbol] | [symbol] | [symbol] |
| [symbol] | | [symbol] | | | | |
| [symbol] | | [symbol] | | | | |
| [symbol] | | [symbol] | [symbol] | [symbol] | | [symbol] |
| [symbol] | | [symbol] | [symbol] | | | |

[symbol]

[symbol]

indicates data missing or illegible when filed

TABLE 4
Shared front layer interfaces with E2ecto. E2 interface
residues shared by hcab55, hcab64, and HEPC74 bNAbs.
List of shared heavy chain (chain H)-E2ecto [symbol] (chain C)
interface residues between HEPC74, hcab55, and hcab64

| E2ecto [symbol] | E2 region | Bond |
|---|---|---|
| C:LEU 427 | FL | |
| C:ASN 428 | FL | |
| C:CYS 429 | FL | H |
| C:ASN 430 | FL | |
| C:ASP 431 | FL | HS or H |
| C:HIS 434 | FL | H |
| C:THR 435 | FL | H |
| C:GLY 436 | FL | |
| C:PHE 437 | FL | |
| C:LEU 438 | FL | |
| C:ALA 439 | FL | |
| C:ALA 440 | FL | |
| C:PHE 442 | FL | |
| C:TYR 443 | FL | |
| C:HIS 445* | FL | |
| C:LYS 446* | FL | HS or H |
| C:PHE 447 | FL | |
| C:ASN 448 | FL | H |
| C:TRP 529 | CD [symbol] 1bl | |
| C:GLU 531 | CD [symbol] 1bl | HS |
| C:TRP 616 | BL | |
| C:PRO 619 | BL | |

*HIS 445 and LYS 446 also form H bonds with non-front-layer bNAb hcab40 [symbol]
indicates data missing or illegible when filed

TABLE 5
hcab40 and hcab17 interfaces with E2

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | [symbol] | [symbol] |
| [symbol] | | [symbol] | [symbol] | [symbol] | | [symbol] |
| [symbol] | [symbol] | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | | [symbol] |
| [symbol] | [symbol] | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | [symbol] | [symbol] |
| [symbol] | [symbol] | [symbol] | [symbol] | [symbol] | [symbol] | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | | [symbol] |
| | | | [symbol] | [symbol] | | [symbol] |
| | | | [symbol] | [symbol] | | [symbol] |
| | | | [symbol] | [symbol] | | [symbol] |
| [symbol] | | [symbol] | | | | [symbol] |

[symbol]

| [symbol] | [symbol] | [symbol] | [symbol] | [symbol] |
|---|---|---|---|---|
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | | [symbol] |
| [symbol] | | [symbol] | | | | |
| [symbol] | | [symbol] | | | | |
| [symbol] | | [symbol] | [symbol] | | | [symbol] |

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | [symbol] | [symbol] |
| [symbol] | [symbol] | [symbol] | | | | |
| [symbol] | | [symbol] | | | | |
| [symbol] | | [symbol] | | | | |
| [symbol] | [symbol] | [symbol] | [symbol] | [symbol] | [symbol] | [symbol] |
| [symbol] | | [symbol] | | | | |
| [symbol] | | [symbol] | | | | |
| [symbol] | | [symbol] | [symbol] | [symbol] | | [symbol] |
| [symbol] | | [symbol] | [symbol] | | | [symbol] |
| [symbol] | | | | | | |
| [symbol] | | | | | | |
| [symbol] | [symbol] | [symbol] | [symbol] | [symbol] | [symbol] | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | [symbol] | [symbol] |
| [symbol] | | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| [symbol] | [symbol] | [symbol] | [symbol] | [symbol] | | [symbol] |
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | [symbol] | [symbol] |
| | | | [symbol] | [symbol] | | [symbol] |
| | | | [symbol] | [symbol] | | [symbol] |
| [symbol] | | [symbol] | | | | [symbol] |

[symbol]

| [symbol] | [symbol] | [symbol] | [symbol] | [symbol] |
|---|---|---|---|---|
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |

[symbol]

| [symbol] | [symbol] | [symbol] | [symbol] | [symbol] |
|---|---|---|---|---|
| [symbol] | [symbol] | [symbol] | [symbol] | [symbol] |

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | | [symbol] |
| [symbol] | | [symbol] | [symbol] | [symbol] | | [symbol] |
| [symbol] | | [symbol] | [symbol] | [symbol] | [symbol] | [symbol] |
| [symbol] | [symbol] | [symbol] | [symbol] | [symbol] | | [symbol] |
| [symbol] | | [symbol] | [symbol] | [symbol] | [symbol] | [symbol] |
| [symbol] | | [symbol] | [symbol] | [symbol] | | [symbol] |
| | | | [symbol] | [symbol] | | [symbol] |
| | | | [symbol] | [symbol] | | [symbol] |
| | | | [symbol] | [symbol] | | [symbol] |
| [symbol] | | [symbol] | [symbol] | | | [symbol] |

[symbol]

| [symbol] | [symbol] | [symbol] | [symbol] | [symbol] |
|---|---|---|---|---|
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |

[symbol]

| [symbol] | [symbol] | [symbol] | [symbol] | [symbol] |
|---|---|---|---|---|
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |

[symbol]

| [symbol] | [symbol] | [symbol] | [symbol] | [symbol] | [symbol] | [symbol] |
|---|---|---|---|---|---|---|
| [symbol] | | [symbol] | [symbol] | [symbol] | [symbol] | [symbol] |
| [symbol] | | [symbol] | | | | |
| [symbol] | [symbol] | [symbol] | | | | |
| [symbol] | | [symbol] | | | | |
| [symbol] | [symbol] | [symbol] | [symbol] | [symbol] | [symbol] | [symbol] |
| [symbol] | | [symbol] | | | | |
| [symbol] | | [symbol] | [symbol] | [symbol] | | [symbol] |
| [symbol] | | [symbol] | [symbol] | | | [symbol] |

[symbol]

[symbol]

indicates data missing or illegible when filed

### DISCUSSION

In this study, a mixture of three soluble E2 ectodomain proteins was used to perform BCR-seq and culture of E2-reactive B cells from an Elite Neutralizer who spontaneously cleared multiple HCV infections. 55 E2-reactive mAbs were isolated, including 29 bNAbs, and their potency, neutralization breadth, epitope targets, and longitudinal evolution was characterized. It was demonstrated that there is a preference but no necessity for V_{H}1-69 or D2-2/D2-15 gene segment usage among HCV bNAbs, with the highest breadth bNAbs using a wide range of gene segments. These bNAbs targeted the conserved E2 front-layer (FRLY) as well as a variety of novel epitopes, including a neutralizing epitope in the back layer of E2. Back layer-reactive mAbs were previously thought to be non-neutralizing (Hadlock et al., 2000). An extraordinarily broadly neutralizing antibody, designated hcab05, was identified which appears to bind near the stalk of E2. Unlike other bNAbs in this class, such as AR4A, hcab05 does not require E1 stabilization of E2 for binding. The existence of multiple neutralizing epitopes in front and back layers of E2 suggests the possibility of a synergistic antibody response between bNAbs that utilize multiple V_{H}-genes. Additionally, through longitudinal BCR-seq analysis, convergent evolution of multiple FRLY bNAb lineages was observed, identifying a mechanism by which bNAbs can acquire neutralizing breadth regardless of their germline V_{H} or D gene usage. These findings highlight the remarkable plasticity of the HCV bNAb response and could guide vaccine development.

In a recent study by Weber et al. (Weber et al., 2022b), bNAbs primarily targeted the E2-front-layer and were almost exclusively reliant on the V_{H}1-69 gene segment. In contrast, here bNAbs were isolated utilizing a wide range of V_{H} gene segments, targeting the front layer as well as a wide variety of other novel E2 epitopes. Numerous differences in experimental approach might explain these discrepancies. First, Weber et al. isolated bNAbs from individuals with chronic HCV infection, whereas the bNAbs here were isolated from a person who spontaneously cleared multiple infections. Second, Weber et al. isolated E2-reactive B cells using a single-variant E2 ‘core’ protein fragment with deleted variable domains and mutated glycosylation sites. In contrast, here a mixture of unmutated, full-length, antigenically diverse E2 ectodomain proteins was used.

These findings stand in contrast to studies of HIV-1 infection demonstrating that most bNAbs use a limited set of V_{H}-genes, including V_{H}1-69. In addition, many HIV bNAbs are extensively somatically mutated (~60% homology to germline) with unusual features like exceptionally long CDRH3s (Kumar et al., 2020, Chen et al., 2019). This narrow genetic path to HIV bNAb induction has led to the use of complex, sequential vaccine strategies that have thus far failed to reliably induce bNAbs. In addition to diverse V_{H} and D-gene usage, it was found that HCV bNAbs are not extensively somatically mutated (>87% homology to germline), nor do they have exceptionally long CDRH3s (median <20 amino acids). Thus, the bNAb response against HCV resembles a more typical B cell response to pathogens like influenza or SARS-CoV-2 that should be inducible with already proven vaccine platforms and strategies.

The findings here are also useful for rational HCV vaccine antigen design. Convergent evolution points to a set of BCR amino acids that are of particular importance for neutralizing breadth. Through analysis of bNAb-E2 structures, the specific E2 residues that form contacts with these convergent bNAb positions were identified. Stabilization or optimized presentation of these E2 residues could provide an avenue for enhancement of HCV vaccine antigens. In addition, since the data confirm that V_{H}1-69 or D2-15 gene segments are not required for bNAb induction, animals that do not express orthologs of these gene segments, including mice, should be useful for preclinical evaluation of vaccine candidates.

In conclusion, this study demonstrates that targeting of a wide variety of E2 epitopes and convergent evolution of antibodies targeting the E2 front-layer allows genetically diverse human antibodies to acquire a broadly neutralizing phenotype. Structural studies of these bNAbs in complex with E2 define conserved bNAb-E2 interactions, identifying residues in E2 that could be stabilized or optimally exposed in rationally-designed vaccine antigens. Together, these findings support the feasibility of an HCV vaccine, and provide a roadmap for HCV vaccine development.

Further discussion: In one aspect, we used a mixture of three soluble E2 ectodomain proteins to perform BCR-seq and culture of E2-reactive B cells from an EN who spontaneously cleared multiple HCV infections. We isolated 55 E2-reactive mAbs, including 29 bNAbs, and characterized their neutralization breadth, epitope targets, and longitudinal evolution. We demonstrated that there is a preference but no necessity for V_{H}1-69 or D2-2/D2-15 gene segment usage among HCV bNAbs, with the highest breadth bNAbs using a wide range of gene segments. These bNAbs targeted the conserved E2 FRLY as well as a variety of additional epitopes, including neutralizing epitopes in the b sandwich and back layer of E2. Both b sandwich and back layer-reactive mAbs were previously thought to be non-neutralizing. 18,37 The existence of multiple neutralizing epitopes suggests the possibility of a synergistic antibody response between bNAbs that target these sites. Additionally, through longitudinal BCR-sequence analysis, we observed convergent evolution of multiple FRLY bNAb lineages, identifying a mechanism by which bNAbs can acquire neutralizing breadth regardless of their germline VH-gene usage. These findings highlight the plasticity of the HCV bNAb response and could guide vaccine development.

In a recent study by Weber et al., 15 bNAbs primarily targeted the E2-FRLY and were almost exclusively reliant on the VH1-69 gene segment. We also found that utilization of VH1-69 strongly favored broad neutralizing activity, but in contrast with Weber et al., we isolated bNAbs utilizing a wide range of VH-gene segments, targeting the FRLY as well as a variety of other additional E2 epitopes. In our work, we used a mixture of three soluble E2 ectodomain proteins to perform BCR-seq and culture of E2-reactive B cells from an EN who spontaneously cleared multiple HCV infections. We isolated 55 E2-reactive mAbs, including 29 bNAbs, and characterized their neutralization breadth, epitope targets, and longitudinal evolution. Numerous differences in experimental approach might explain these discrepancies. First, Weber et al. isolated bNAbs from individuals with chronic HCV infection, whereas our bNAbs were isolated from a person with an effective antibody response who spontaneously cleared multiple infections. Second, Weber et al. isolated E2-reactive B cells using a single-variant E2 protein fragment with deleted variable domains and mutated glycosylation sites. By contrast, we used a mixture of unmutated, full-length, antigenically diverse E2 ectodomain proteins.

These findings stand in contrast to studies of HIV-1 infection demonstrating that most bNAbs use a limited set of VH genes, including VH1-69. In addition, many HIV bNAbs are extensively somatically mutated (_60% homology to germ line) with unusual features like exceptionally long CDRH3s.21,22 This narrow genetic path to HIV bNAb induction has led to the use of complex, sequential vaccine strategies that have thus far failed to reliably induce bNAbs. We found that in addition to diverse VH- and D-gene usage, HCV bNAbs are not extensively somatically mutated (>87% homology to germ line), nor do they have exceptionally long CDRH3s (median <20 amino acids). Thus, the bNAb response against HCV resembles a more typical B cell response to pathogens like influenza or SARS-COV-2 that should be inducible with already proven vaccine platforms and strategies.

Our findings are also useful for rational HCV vaccine antigen design. Convergent evolution points to a set of BCR amino acids that are of particular importance for neutralizing breadth. Through analysis of bNAb-E2 structures, we have identified the specific E2 residues that form contacts with these convergent bNAb positions. Stabilization or optimized presentation of these E2 residues could provide an avenue for enhancement of HCV vaccine antigens. In addition, since our data confirm that VH1-69 or D2-15 gene segments are not required for bNAb induction, animals that do not express orthologs of these gene segments, including mice, may be useful for preclinical evaluation of vaccine candidates.

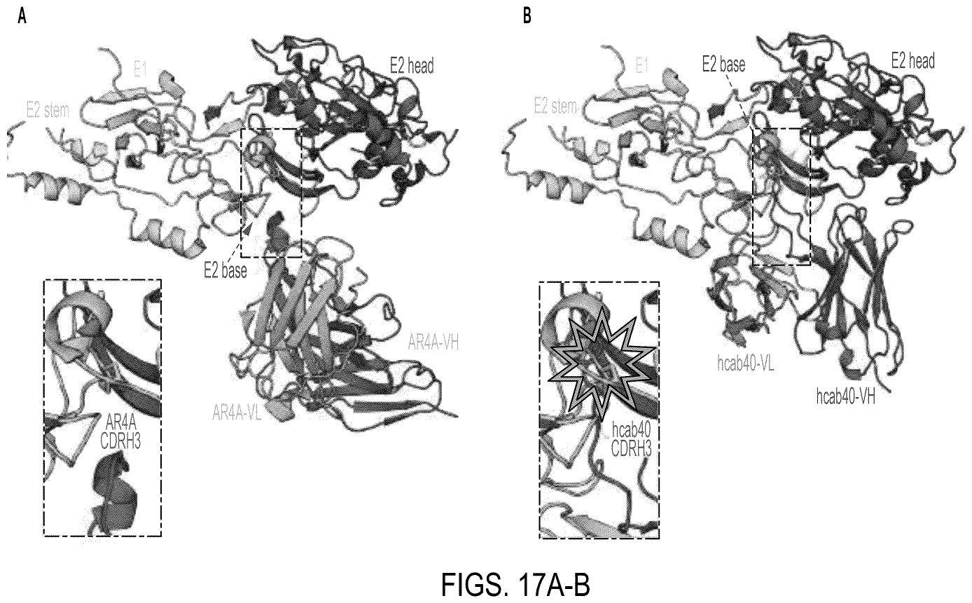

Finally, it is not clear yet whether E2-based or E1E2-based immunogens would fulfill the requirements of an effective HCV vaccine. 40,41 One theoretical advantage of E1E2 immunogens over E2 immunogens is the ability to elicit AR4A-like bNAbs that recognize the conformationally sensitive epitopes near the stem of E2, a region that is stabilized by interactions with E1. Unfortunately, purification of intact E1E2 heterodimers is difficult since the majority of transiently expressed E1E2 forms disulfide-crosslinked aggregates. 42 By contrast, soluble E2 glycoproteins can be expressed at high levels in monomeric form. In this study, we identified a cluster of potent bNAbs, including an extraordinarily bNAb, designated hcab05, that binds to the back layer of E2. Structural analysis of a representative bNAb hcab40 from the back layer binding group indicated that its epitope overlaps with the AR4A epitope in E1E2 (FIG. 17). However, unlike AR4A, hcab40 and other hcabs that bind to the back layer of E2 do not require E1

Results disclosed herein demonstrate among other things that targeting of a wide variety of E2 epitopes and convergent evolution of antibodies targeting the E2 FRLY allows genetically diverse human antibodies to acquire a broadly neutralizing phenotype. Structural studies of these bNAbs in complex with E2 define conserved bNAb-E2 interactions, identifying residues in E2 that could be stabilized or optimally exposed in rationally designed vaccine antigens. Together, these findings show the feasibility of an HCV vaccine and support the development of vaccines that can induce bNAbs targeting multiple E2 epitopes.

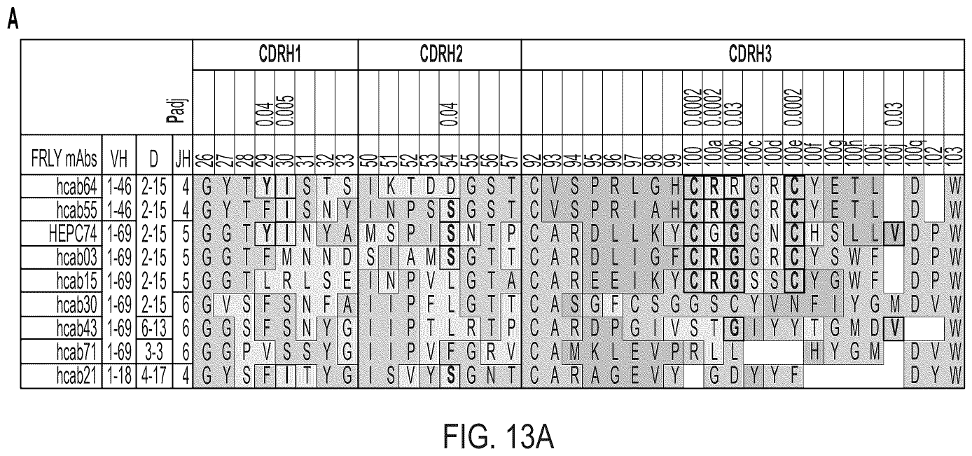

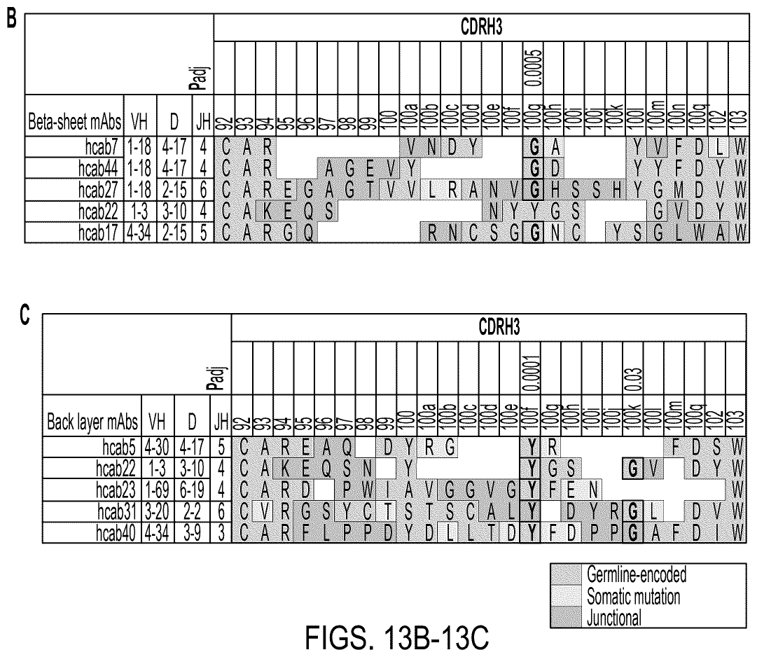

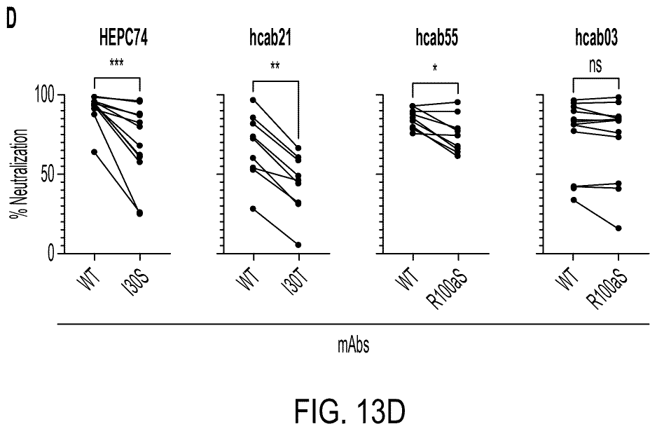

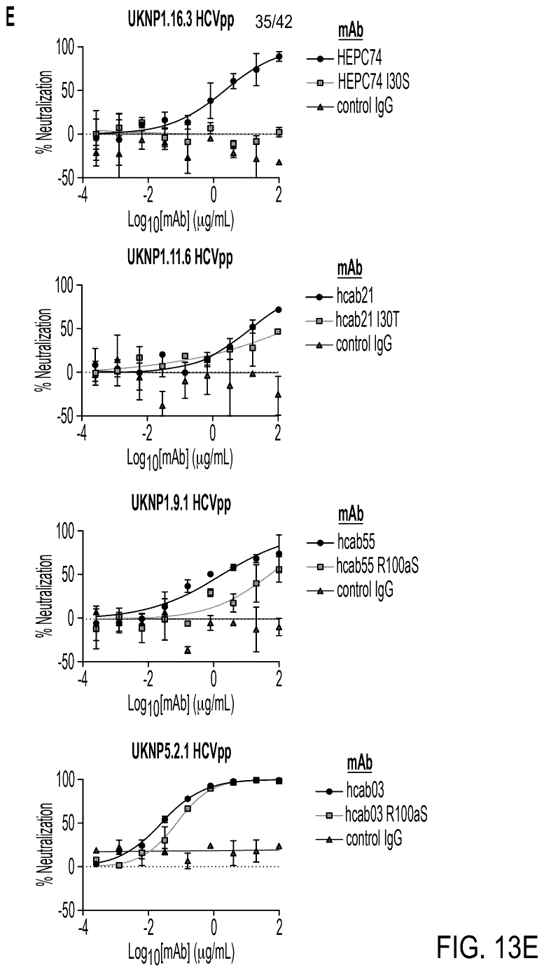

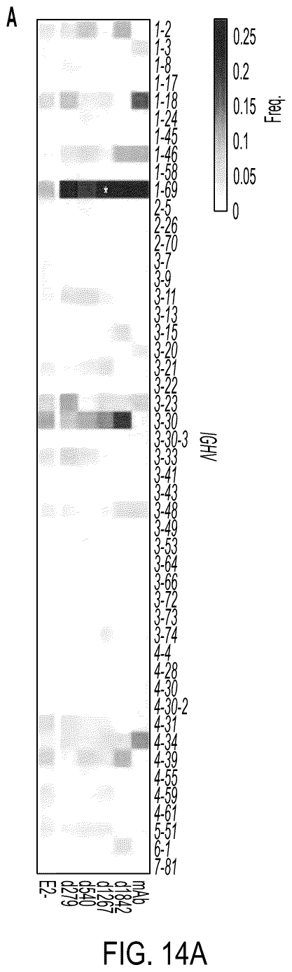

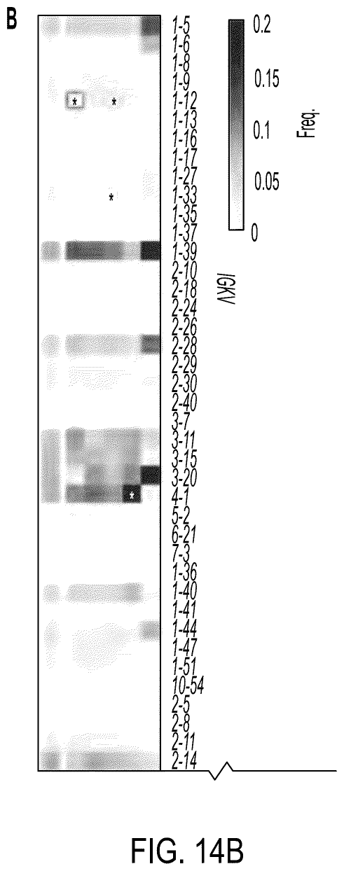

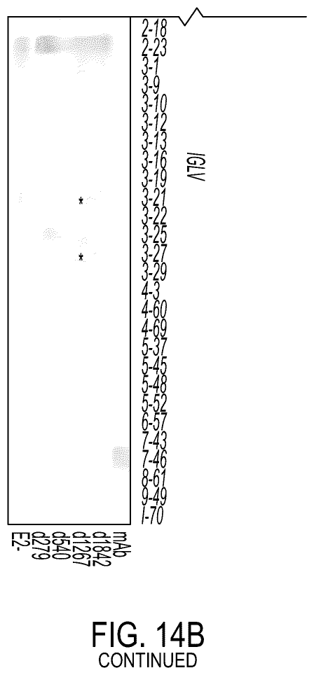

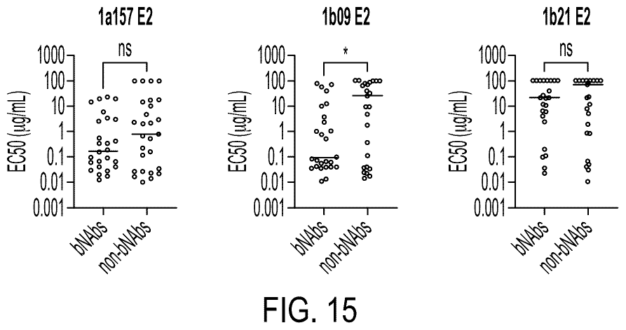

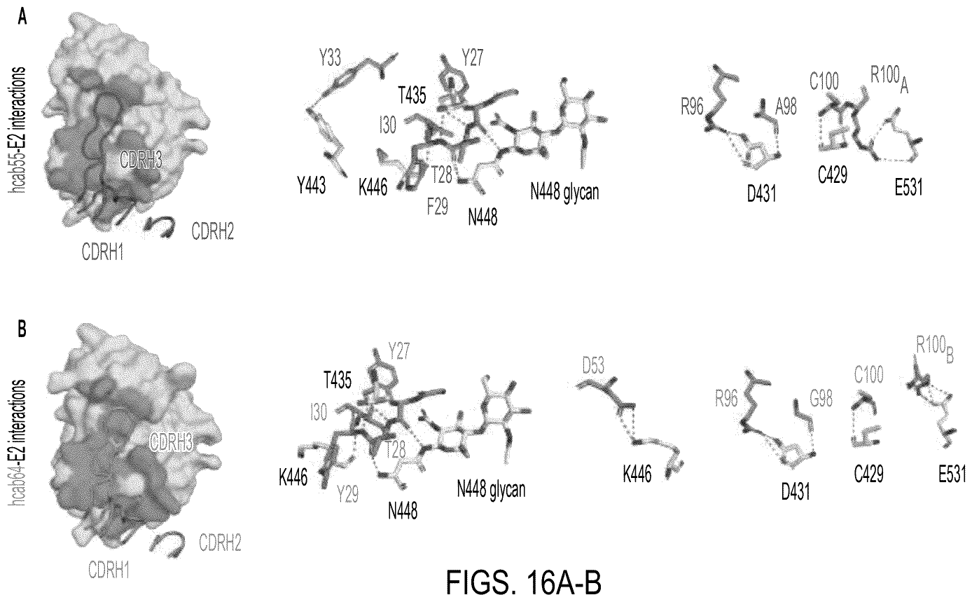

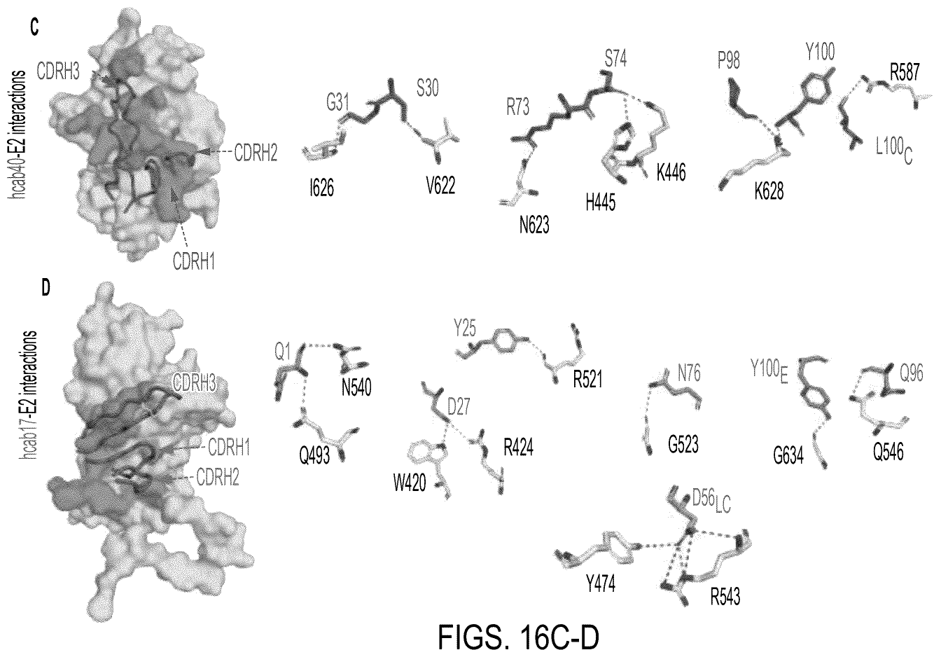

Results and disclosure set forth herein is also set forth in Ogeng et al., “Convergent evolution and targeting of diverse E2 epitopes by human broadly neutralizing antibodies are associated with HCV clearance”, Immunity, 57, 890-903, Apr. 9, 2024, which is incorporated herein by reference in its entirety. See therein (Ogeng et al., Immunity 57. 890, 2024) for FIGS. 12-17 set forth herein (including color versions thereof) and other figures.

References: (references above are referred to the document listed below either by reference number or by author and year of publication of cited document):
1. van der Meer, A. J., Veldt, B. J., Feld, J. J., Wedemeyer, H., Dufour, J. F., Lammert, F., Duarte-Rojo, A., Heathcote, E. J., Manns, M. P., Kuske, L., et al. (2012). Association between sustained virological response and all-cause mortality among patients with chronic hepatitis C and advanced hepatic fibrosis. JAMA 308, 2584-2593.
2. Davis, G. L., Alter, M. J., El-Serag, H., Poynard, T., and Jennings, L. W. (2010). Aging of hepatitis C virus (HCV)-infected persons in the United States: a multiple cohort model of HCV prevalence and disease progression. Gastroenterology 138, 513-521.
3. Centers for Disease Control and Prevention (2018). Viral Hepatitis Surveillance Report 2018-Hepatitis C. https://www.cdc.gov/hepatitis/statistics/SurveillanceRpts.htm.
4. Suryaprasad, A. G., White, J. Z., Xu, F., Eichler, B. A., Hamilton, J., Patel, A., Hamdounia, S. B., Church, D. R., Barton, K., Fisher, C., et al. (2014). Emerging epidemic of hepatitis C virus infections among young non-urban persons who inject drugs in the United States, 2006-2012. Clin. Infect. Dis. 59, 1411-1419.
5. Osburn, W. O., Fisher, B. E., Dowd, K. A., Urban, G., Liu, L., Ray, S. C., Thomas, D. L., and Cox, A. L. (2010). Spontaneous control of primary hepatitis C virus infection and immunity against persistent reinfection. Gastroenterology 138, 315-324.
6. Thomas, D. L., Thio, C. L., Martin, M. P., Qi, Y., Ge, D., O'Huigin, C., Kidd, J., Kidd, K., Khakoo, S. I., Alexander, G., et al. (2009). Genetic variation in IL28B and spontaneous clearance of hepatitis C virus. Nature 461, 798-801.
7. Osburn, W. O., Snider, A. E., Wells, B. L., Latanich, R., Bailey, J. R., Thomas, D. L., Cox, A. L., and Ray, S. C. (2014). Clearance of hepatitis C infection is associated with the early appearance of broad neutralizing antibody responses. Hepatology 59, 2140-2151.
8. Pestka, J. M., Zeisel, M. B., Bl€aser, E., Sch€urmann, P., Bartosch, B., Cosset, F. L., Patel, A. H., Meisel, H., Baumert, J., Viazov, S., et al. (2007). Rapid induction of virus-neutralizing antibodies and viral clearance in a single-source outbreak of hepatitis C. Proc. Natl. Acad. Sci. USA 104, 6025-6030.
9. Raghuraman, S., Park, H., Osburn, W. O., Winkelstein, E., Edlin, B. R., and Rehermann, B. (2012). Spontaneous clearance of chronic hepatitis C virus infection is associated with appearance of neutralizing antibodies and reversal of T-cell exhaustion. J. Infect. Dis. 205, 763-771.
10. Kinchen, V. J., Zahid, M. N., Flyak, A. I., Soliman, M. G., Learn, G. H., Wang, S., Davidson, E., Doranz, B. J., Ray, S. C., Cox, A. L., et al. (2018). Broadly neutralizing antibody mediated clearance of human hepatitis C virus infection. Cell Host Microbe 24, 717-730.e5.
11. Houghton, M. (2011). Prospects for prophylactic and therapeutic vaccines against the hepatitis C viruses. Immunol. Rev. 239, 99-108.
12. Liang, T. J. (2013). Current progress in development of hepatitis C virus vaccines. Nat. Med. 19, 869-878.
13. Frey, S. E., Houghton, M., Coates, S., Abrignani, S., Chien, D., Rosa, D., Pileri, P., Ray, R., Di Bisceglie, A. M., Rinella, P., et al. (2010). Safety and immunogenicity of HCV E1E2 vaccine adjuvanted with MF59 administered to healthy adults. Vaccine 28, 6367-6373.
14. de Jong, Y. P., Dorner, M., Mommersteeg, M. C., Xiao, J. W., Balazs, A. B., Robbins, J. B., Winer, B. Y., Gerges, S., Vega, K., Labitt, R. N., et al. (2014). Broadly neutralizing antibodies abrogate established hepatitis C virus infection. Sci. Transl. Med. 6, 254ra129.
15. Weber, T., Potthoff, J., Bizu, S., Labuhn, M., Dold, L., Schoofs, T., Horning, M., Ercanoglu, M. S., Kreer, C., Gieselmann, L., et al. (2022). Analysis of antibodies from HCV elite neutralizers identifies genetic determinants of broad neutralization. Immunity 55, 341-354.e7.
16. Law, M., Maruyama, T., Lewis, J., Giang, E., Tarr, A. W., Stamataki, Z., Gastaminza, P., Chisari, F. V., Jones, I. M., Fox, R. I., et al. (2008). Broadly neutralizing antibodies protect against hepatitis C virus quasispecies challenge. Nat. Med. 14, 25-27.
17. Bailey, J. R., Flyak, A. I., Cohen, V. J., Li, H., Wasilewski, L. N., Snider, A. E., Wang, S., Learn, G. H., Kose, N., Loerinc, L., et al. (2017). Broadly neutralizing antibodies with few somatic mutations and hepatitis C virus clearance. JCI Insight 2, e92872.
18. Hadlock, K. G., Lanford, R. E., Perkins, S., Rowe, J., Yang, Q., Levy, S., Pileri, P., Abrignani, S., and Foung, S. K. (2000). Human monoclonal antibodies that inhibit binding of hepatitis C virus E2 protein to CD81 and recognize conserved conformational epitopes. J. Virol. 74, 10407-10416.
19. Chan, C. H., Hadlock, K. G., Foung, S. K., and Levy, S. (2001). V(H) 1-69 gene is preferentially used by hepatitis C virus-associated B cell lymphomas and by normal B cells responding to the E2 viral antigen. Blood 97, 1023-1026.
20. Tzarum, N., Giang, E., Kong, L., He, L., Prentoe, J., Augestad, E., Hua, Y., Castillo, S., Lauer, G. M., Bukh, J., et al. (2019). Genetic and structural insights into broad neutralization of hepatitis C virus by human VH1-69 antibodies. Sci. Adv. 5, eaav1882.
21. Chen, F., Tzarum, N., Wilson, I. A., and Law, M. (2019). VH1-69 antiviral broadly neutralizing antibodies: genetics, structures, and relevance to rational vaccine design VH1. Curr. Opin. Virol. 34, 149-159.
22. Kumar, S., Ju, B., Shapero, B., Lin, X., Ren, L., Zhang, L., Li, D., Zhou, Z., Feng, Y., Sou, C., et al. (2020). A VH1-69 antibody lineage from an infected Chinese donor potently neutralizes HIV-1 by targeting the V3 glycan Supersite. Sci. Adv. 6, eabb1328.
23. Lang, S., Xie, J., Zhu, X., Wu, N. C., Lerner, R. A., and Wilson, I. A. (2017). Antibody 27F3 broadly targets influenza A Group 1 and 2 hemagglutinins through a further variation in VH1-69 antibody orientation on the HA Stem. Cell Rep. 20, 2935-2943.
24. Lingwood, D., McTamney, P. M., Yassine, H. M., Whittle, J. R., Guo, X., Boyington, J. C., Wei, C. J., and Nabel, G. J. (2012). Structural and genetic basis for development of broadly neutralizing influenza antibodies. Nature 489, 566-570.
25. Smith, S. A., Burton, S. L., Kilembe, W., Lakhi, S., Karita, E., Price, M., Allen, S., and Derdeyn, C. A. (2018). VH1-69 utilizing antibodies are capable of mediating non-neutralizing Fc-mediated effector functions against the transmitted/founder gp120 VH1. Front. Immunol. 9, 3163.
26. Flyak, A. I., Ruiz, S., Colbert, M. D., Luong, T., Crowe, J. E., Jr., Bailey, J. R., and Bjorkman, P. J. (2018). HCV broadly neutralizing antibodies use a CDRH3 disulfide motif to recognize an E2 glycoprotein site that can be targeted for vaccine design. Cell Host Microbe 24, 703-716.e3.
27. Frumento, N., Sinnis-Bourozikas, A., Paul, H. T., Stavrakis, G., Zahid, M. N., Wang, S., Ray, S. C., Flyak, A. I., Shaw, G. M., Cox, A. L., and Bailey, J. R. (2024). Neutralizing antibodies evolve to exploit vulnerable sites in the HCV envelope glycoprotein E2 and mediate spontaneous clearance of infection. Immunity 57, 40-51.e5.
28. Cox, A. L., Netski, D. M., Mosbruger, T., Sherman, S. G., Strathdee, S., Ompad, D., Vlahov, D., Chien, D., Shyamala, V., Ray, S. C., and Thomas, D. L. (2005). Prospective evaluation of community-acquired acute-phase hepatitis C virus infection. Clin. Infect. Dis. 40, 951-958.
29. Salas, J. H., Urbanowicz, R. A., Guest, J. D., Frumento, N., Figueroa, A., Clark, K. E., Keck, Z., Cowton, V. M., Cole, S. J., Patel, A. H., et al. (2022). An antigenically diverse, representative panel of envelope glycoproteins for hepatitis C virus vaccine development. Gastroenterology 162, 562-574.
30. Ogega, C. O., Skinner, N. E., Flyak, A. I., Clark, K. E., Board, N. L., Bjorkman, P. J., Crowe, J. E., Jr., Cox, A. L., Ray, S. C., and Bailey, J. R. (2022). B cell overexpression of FCRL5 and PD-1 is associated with low antibody titers in HCV infection. PLOS Pathog. 18, e1010179.
31. Keck, Z. Y., Li, T. K., Xia, J., Bartosch, B., Cosset, F. L., Dubuisson, J., and Foung, S. K. (2005). Analysis of a highly flexible conformational immunogenic domain a in hepatitis C virus E2. J. Virol. 79, 13199-13208.
32. Colbert, M. D., Flyak, A. I., Ogega, C. O., Kinchen, V. J., Massaccesi, G., Hernandez, M., Davidson, E., Doranz, B. J., Cox, A. L., Crowe, J. E., Jr., and Bailey, J. R. (2019). Broadly neutralizing antibodies targeting new sites of vulnerability in hepatitis C virus E1E2. J. Virol. 93. e02070-e02018.
33. Keck, Z.-Y., Li, T.-K., Xia, J., Gal-Tanamy, M., Olson, O., Li, S. H., Patel, A. H., Ball, J. K., Lemon, S. M., and Foung, S. K. H. (2008). Definition of a conserved immunodominant domain on hepatitis C virus E2 glycoprotein by neutralizing human monoclonal antibodies. J. Virol. 82, 6061-6066.
34. Keck, Z. Y., Xia, J., Wang, Y., Wang, W., Krey, T., Prentoe, J., Carlsen, T., Li, A. Y., Patel, A. H., Lemon, S. M., et al. (2012). Human monoclonal antibodies to a novel cluster of conformational epitopes on HCV e2 with resistance to neutralization escape in a genotype 2a isolate. PLOS Pathog. 8, e1002653.
35. Broering, T. J., Garrity, K. A., Boatright, N. K., Sloan, S. E., Sandor, F., Thomas, W. D., Jr., Szabo, G., Finberg, R. W., Ambrosino, D. M., and Babcock, G. J. (2009). Identification and characterization of broadly neutralizing human monoclonal antibodies directed against the E2 envelope glycoprotein of hepatitis C virus. J. Virol. 83, 12473-12482.
36. Kong, L., Giang, E., Nieusma, T., Kadam, R. U., Cogburn, K. E., Hua, Y., Dai, X., Stanfield, R. L., Burton, D. R., Ward, A. B., et al. (2013). Hepatitis C virus E2 envelope glycoprotein core structure. Science 342, 1090-1094.
37. Law, M. (2021). Antibody responses in hepatitis C infection. Cold Spring Harb. Perspect. Med. 11, a036962.
38. Kumar, A., Hossain, R. A., Yost, S. A., Bu, W., Wang, Y., Dearborn, A. D., Grakoui, A., Cohen, J. I., and Marcotrigiano, J. (2021). Structural insights into hepatitis C virus receptor binding and entry. Nature 598, 521-525.
39. Torrents de la Pena, A., Sliepen, K., Eshun-Wilson, L., Newby, M. L., Allen, J. D., Zon, I., Koekkoek, S., Chumbe, A., Crispin, M., Schinkel, J., et al. (2022). Structure of the hepatitis C virus E1E2 glycoprotein complex. Science 378, 263-269. https:/doi.org/10.1126/science.abn9884
40. Torresi, J. (2017). The rationale for a preventative HCV virus-like particle (VLP) vaccine. Front. Microbiol. 8, 2163.
41. Bailey, J. R., Barnes, E., and Cox, A. L. (2019). Approaches, progress, and challenges to hepatitis C vaccine development. Gastroenterology 156, 418-430.
42. Guest, J. D., Wang, R., Elkholy, K. H., Chagas, A., Chao, K. L., Cleveland, T. E. t., Kim, Y. C., Keck, Z. Y., Marin, A., Yunus, A. S., et al. (2021). Design of a native-like secreted form of the hepatitis C virus E1E2 heterodimer. Proc. Natl. Acad. Sci. USA 118, e2015149118.
43. Cox, A. L., Mosbruger, T., Lauer, G. M., Pardoll, D., Thomas, D. L., and Ray, S. C. (2005). Comprehensive analyses of CD8+ T cell responses during longitudinal study of acute human hepatitis C. Hepatology 42, 104-112.
44. Nakabayashi, H., Taketa, K., Yamane, T., Miyazaki, M., Miyano, K., and Sato, J. (1984). Phenotypical stability of a human hepatoma cell line, HuH-7, in long-term culture with chemically defined medium. Gan 75, 151-158.
45. Kalemera, M. D., Capella-Pujol, J., Chumbe, A., Underwood, A., Bull, R. A., Schinkel, J., Sliepen, K., and Grove, J. (2021). Optimized cell systems for the investigation of hepatitis C virus E1E2 glycoproteins. J. Gen. Virol. 102, jgv001512.
46. Huang, J., Doria-Rose, N. A., Longo, N. S., Laub, L., Lin, C.-L., Turk, E., Kang, B. H., Migueles, S. A., Bailer, R. T., Mascola, J. R., and Connors, M. (2013). Isolation of human monoclonal antibodies from peripheral blood B cells. Nat. Protoc. 8, 1907-1915.
47. Tiller, T., Meffre, E., Yurasov, S., Tsuiji, M., Nussenzweig, M. C., and Wardemann, H. (2008). Efficient generation of monoclonal antibodies from single human B cells by single cell RT-PCR and expression vector cloning. J. Immunol. Methods 329, 112-124.
48. Adams, P. D., Afonine, P. V., Bunko' czi, G., Chen, V. B., Davis, I. W., Echols, N., Headd, J. J., Hung, L. W., Kapral, G. J., Grosse-Kunstleve, R. W., et al. (2010). Phenix: a comprehensive Python-based system for macromolecular structure solution. Acta Crystallogr. D Biol. Crystallogr. 66, 213-221.
49. Emsley, P., and Cowtan, K. (2004). Coot: model-building tools for molecular graphics. Acta Crystallogr. D Biol. Crystallogr. 60, 2126-2132. 50. Krissinel, E., and Henrick, K. (2007). Inference of macromolecular assemblies from crystalline state. J. Mol. Biol. 372, 774-797.
51. Burke, K. P., Munshaw, S., Osburn, W. O., Levine, J., Liu, L., Sidney, J., Sette, A., Ray, S. C., and Cox, A. L. (2012). Immunogenicity and cross-reactivity of a representative ancestral sequence in HCV infection. J. Immunol. 188, 5177-5188.
52. Chattergoon, M. A., Latanich, R., Quinn, J., Winter, M. E., Buckheit, R. W., 3rd, Blankson, J. N., Pardoll, D., and Cox, A. L. (2014). HIV and HCV activate the inflammasome in monocytes and macrophages via endosomal toll-like receptors without induction of Type 1 interferon. PLOS Pathog. 10, e1004082.
53. El-Diwany, R., Cohen, V. J., Mankowski, M. C., Wasilewski, L. N., Brady, J. K., Snider, A. E., Osburn, W. O., Murrell, B., Ray, S. C., and Bailey, J. R. (2017). Extra-epitopic hepatitis C virus polymorphisms confer resistance to broadly neutralizing antibodies by modulating binding to scavenger receptor B1. PLOS Pathog. 13, e1006235.
54. Bailey, J. R., Urbanowicz, R. A., Ball, J. K., Law, M., and Foung, S. K. H. (2019). Standardized method for the study of antibody neutralization of HCV pseudoparticles (HCVpp). Methods Mol. Biol. 1911, 441-450.
55. Wasilewski, L. N., Ray, S. C., and Bailey, J. R. (2016). Hepatitis C virus resistance to broadly neutralizing antibodies measured using replication competent virus and pseudoparticles. J. Gen. Virol. 97, 2883-2893.
56. Pierce, B. G., Keck, Z. Y., Lau, P., Fauvelle, C., Gowthaman, R., Baumert, T. F., Fuerst, T. R., Mariuzza, R. A., and Foung, S. K. H. (2016). Global mapping of antibody recognition of the hepatitis C virus E2 glycoprotein: implications for vaccine design. Proc. Natl. Acad. Sci. USA 113, E6946-E6954.
57. Flyak, A. I., Ilinykh, P. A., Murin, C. D., Garron, T., Shen, X., Fusco, M. L., Hashiguchi, T., Bornholdt, Z. A., Slaughter, J. C., Sapparapu, G., et al. (2015). Mechanism of human antibody-mediated neutralization of Marburg virus. Cell 160, 893-903.
58. Battye, T. G., Kontogiannis, L., Johnson, O., Powell, H. R., and Leslie, A. G. (2011). iMOSFLM: a new graphical interface for diffraction-image processing with MOSFLM. Acta Crystallogr. D Biol. Crystallogr. 67, 271-281.
59. Evans, P. R., and Murshudov, G. N. (2013). How good are my data and what is the resolution? Acta Crystallogr. D Biol. Crystallogr. 69, 1204-1214.
60. Chen, V. B., Arendall, W. B., 3rd, Headd, J. J., Keedy, D. A., Immormino, R. M., Kapral, G. J., Murray, L. W., Richardson, J. S., and Richardson, D. C. (2010). MolProbity: all-atom structure validation for macromolecular crystallography. Acta Crystallogr. D Biol. Crystallogr. 66, 12-21.

### OTHER EMBODIMENTS

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

1. A method of producing neutralizing antibodies to hepatitis virus comprising:
isolating antigenically diverse proteins from the hepatitis virus,
incubating peripheral blood mononuclear cells (PBMCs) with a mixture of the isolated antigenic diverse proteins,
sorting the PBMCs to obtain antigen reactive class-switched memory B cells and non antigen reactive class-switched memory B cells,
seeding the antigen reactive class-switched memory B cell cells in limiting dilutions in cell culture wells wherein each cell culture well comprises at least one antigen reactive class-switched memory B cell,
stimulating the class-switched memory B cell cultures with one or more cytokines and antigen,
isolating supernatants from the antigen reactive class-switched memory B cell cultures, and,
producing neutralizing antibodies to hepatitis virus.

2. The method of claim 1, wherein the hepatitis virus comprises hepatitis A virus (HAV), hepatitis B virus (HBV), hepatitis C virus (HCV), hepatitis D virus (HDV), or hepatitis E virus (HEV).

3. The method of claim 1, wherein the virus is HCV.

4. The method of claim 4, wherein the antigenically diverse proteins isolated from the HCV comprise E2 ectodomain proteins.

5. The method of claim 4, wherein the antigenically diverse proteins E2 ectodomain proteins are isolated from one or more HCV genotypes comprising genotypes 1-7 or 8.

6. The method of claim 5, wherein the HCV antigenically diverse E2 ectodomain proteins are isolated from one or more HCV subtypes.

7. The method of claim 6, wherein the one or more HCV subtypes comprise subtypes comprise subtypes 1a-1o, 2a-2v, 3a-3k, 4a-4w, 5a, 6a-6xj, 7a-7b or 8a.

8. The method of claim 7, wherein the E2 ectoproteins are isolated from subtype 1.

9. The method of claim 8, wherein E2 ectoproteins are isolated from subtypes 1a, subtype 1b or the combination thereof.

10. The method of claim 1, wherein nucleic acids encoding immunoglobulin variable heavy and light chain sequences isolated from antigen reactive class-switched memory B cells are subjected to RT-PCR and sequenced.

11. The method of claim 10, wherein the immunoglobulin variable sequences are cloned into human IgG kappa (κ) or lambda (λ) expression plasmids.

12. The method of claim 11, wherein the heavy and light chains are co-expressed to generate monoclonal antibodies.

13. The method of claim 11, wherein the monoclonal antibodies comprise immunoglobulin G (IgG) isotypes IgG1, IgG2, IgG3 or IgG4.

14-18. (canceled)

19. An isolated nucleic acid comprising: (i) a variable heavy chain having at least a 90% sequence identity to SEQ ID NOs: 1-57 or 58; or (ii) a variable kappa light (Vκ) chain having at least a 90% sequence identity to SEQ ID NOs: 59-107 or 108; or (iii) a variable lambda light (Vλ) chain having at least a 90% sequence identity to SEQ ID NOs: 109-122 or 123, or combinations thereof.

20. The isolated nucleic acid of claim 19, wherein (i) the variable heavy chain (V_{H}) comprises SEQ ID NOs: 1-57 or 58; or (ii) the variable kappa light (Vκ) chain comprises SEQ ID NOs: 59-107 or 108; or (iii) the variable lambda light (Vλ) chain comprises SEQ ID NOs: 109-122 or 123, or combinations thereof.

21-23. (canceled)

24. The isolated nucleic acid of any one of claim 19 or 20, wherein the isolated nucleic acid comprises any one V_{H} sequence comprising SEQ ID NOs: 1-57 or 58 and any one Vκ sequence comprising SEQ ID NOs: 59-107 or 108.

25. The isolated nucleic acid of any one of claim 19 or 20, wherein the isolated nucleic acid comprises any one V_{H} sequence comprising SEQ ID NOs: 1-57 or 58 and any one Vλ sequence comprising SEQ ID NOs: 109-122 or 123.

26-28. (canceled)

29. A method of preventing or treating a hepatitis C virus (HCV) infection comprising: administering to a subject a therapeutically effective amount of an HCV neutralizing antibody,
thereby, treating the subject.

30. The method of claim 29, wherein the HCV neutralizing antibody comprises a variable heavy chain (V_{H}) comprising SEQ ID NOs: 1-57 or 58 and a variable kappa light (Vκ) chain comprising SEQ ID NOs: 59-107 or 108.

31. The method of claim 29, wherein the HCV neutralizing antibody comprises a variable heavy chain (V_{H}) comprising SEQ ID NOs: 1-57 or 58 and a variable lambda light (Vλ) chain comprising SEQ ID NOs: 109-122 or 123.

32-42. (canceled)


SEQUENCE LISTING (216 sequences)

SEQ ID NO: 1 - DNA, 372 bp, synthetic construct
  source 1..372: mol_type = other DNA
caggtgcagctggtgcagtcaggggctgaggtgaagaagcctggggcctcagtgaaggtttcctgcaagaattctggatacaccctcagtaactctggcatgcattgggtgcgccaggcccccggacaaaggcctgagtggatgggatggataaacgttgccgaaggttacacaaaatattcagagaagttgcaggccagactcaccataaccagtgacacagccgcgaacacagtctacatggagctgagcagcctgagatctgaagactcggctgtgtattactgtgcgagtgttacgacgagacaatggttcgggaggggtgatgcttttgatctctggggccaagggacaatggtcaccgcctcctca

SEQ ID NO: 2 - DNA, 378 bp, synthetic construct
  source 1..378: mol_type = other DNA
caggtgcagctggtgcagtctggggctgaggtgaagaagcctgggtcctcggtgaaggtgtcctgcaaggcctctggaggcaccttcatgaacaatgatatcaactgggtgcgacaggcccctggacaagggcttgagtggatgggagggagcattgctatgtctgggacaacaaactacgcacagaagttccagggcagactgtcgataaccgcggacgattccacgggcacagcctacatggagctgaacagcctgagatctgacgacacggccgtatattactgtgcgagagatctaatcgggttttgtagaggtggaaggtgctactcctggttcgacccctggggccagggaaccctggtcaccgtctcctca

SEQ ID NO: 3 - DNA, 379 bp, synthetic construct
  source 1..379: mol_type = other DNA
caggtccagctggtgcaatctggggatgaggtgaagaagcctgggtcctcggtcaaggtctcctgcaagacttccggaggcaccttcgccatctataccatttcctgggtgcgacaggcccctggagtaggtcttgactggatggggagcatcacccctatggttggcagaacaaaatacgcacagaacttccagggcagagtcacgattaccgcggacacatccacgaacacagtctacatggagctgagcagtctgagctctcaagacacggccatctatttctgtgcgtatcatggatcacaaatgttgggaacctatgacaatgccaactggttcgacccctggggccagggaaccccggtcaccgtctcctcag

SEQ ID NO: 4 - DNA, 367 bp, synthetic construct
  source 1..367: mol_type = other DNA
caggtgcagctgcaggagtccggctcaggactggtgaggccttcacagaccctgtccctcacctgcactgtctctggtggctccatcagcagtgatgcttactcctggaactggatccggcagccaccagggaagggcctggaatggattggtcacatctatcatagtggccgcacctcccacaacccgtccctcaacagtcgcgtcaccatctctgtagacaggtccaagaaccagttctccctgcggctgacctctgtgaccgccgcggacacggccgtctattattgtgccagagaggcccaggactaccgtggctaccgtttcgactcctggggccagggaaccctggtcaccgtctcctcag

SEQ ID NO: 5 - DNA, 354 bp, synthetic construct
  source 1..354: mol_type = other DNA
caggtgcagctggtgcagtctggacctgaggtgaagaagcctggggcctcagtgaaggtctcctgcaaggcctctggttacatctttaccagccatggcatcacctgggtgcgccaggcccctggacaagggcttgagtgcatggggtggatcagtgcttacagtggtcattcaaactctgcacagaacttccagggcagagtcaccatgaccagagacacatccacgggcacagcctacatggaactgaggagcctgagatctgacgacacggccgtctactactgtgcgcgagcgtcagcggaatatggtttggacgtctggggccaagggaccacggtcaccgtctcctca

SEQ ID NO: 6 - DNA, 360 bp, synthetic construct
  source 1..360: mol_type = other DNA
caggtgcagctggtgcagtctggagctgaggtgaagaagcctggggcctcagtgaaggtctcctgcaaggcttctggctacacctttaccacctatggtatcacctgggtgcgacaggcccctggacaagggcttgagtgggtgggatggatcagcgcttacagtggtaacacaaactatgcacaggaggtccagggcagagtcaccctgaccacagacacatccacgaccacatcctacatggaactgaggagcctgagatctgacgacacggccgtgtattactgtgcgagagtgaatgactacggtgcctacgtctttgacctctggggccagggaaccctggtcaccgtctcctca

SEQ ID NO: 7 - DNA, 402 bp, synthetic construct
  source 1..402: mol_type = other DNA
caggtgcagctggtggagtctgggggaggcttggtccagccgggggggtccctgagactctcctgtgcagcctctggattcacctttaatacctattggatgagctgggtccgccaggctccagggaaagggctggagtgggtggccaacataaagcaagatggaagcgagaaatactatgtggactctgtgaagggccgattcaccatctccagagacaacgccaagaactcgctgtatctgcaaatgaacagcctgagagccgaggacacggctgtgtattactgtgcgagggatagacccgcgggaattactatggttcggggaacgactattatagaggaggactactactacggtatggacgtctggggccaagggaccacggtcaccgtctcctca

SEQ ID NO: 8 - DNA, 354 bp, synthetic construct; identical to SEQ ID NO: 5
  source 1..354: mol_type = other DNA

SEQ ID NO: 9 - DNA, 375 bp, synthetic construct
  source 1..375: mol_type = other DNA
caggtgcagctggtggagtctgggggaggcttggtacagcctggggggtccctgagactctcctgtgcagcctctggactcagcttcagtagtttctccattgactgggtccgccaaggtagaggagatagtctggagggggtctcactcactggtattgttgatgacacagttcccaggctccgtgaagggccaattcaccatctccagagaaaacggcacgacctccttgttgtttcatatgaacagcctgagagccggggacacaactgtgtatcactgtacaggagatcggttgcattagtgtgacagtcgtgaaaagtagtcgctcgtcttggctcctggggccagggaaccctggtcaccgtctcctca

SEQ ID NO: 10 - DNA, 381 bp, synthetic construct
  source 1..381: mol_type = other DNA
caggtgcagctggtggagtcggggggaggcttggtacagcctggggggtccctcacagtctcatgtgcagcctctggattctatttcagtggctatagtatgaactgggtccgccaggctccagggaaggggctggagtgggtttcatacattagtagtagtggtagaaccattcactacgcagactctgtgaggggccgattcaccatctccagagacaatgccaagaattcactgtatctgcgaatggacagcctgagagtcgaggacacgtctatctattactgtgcgagagatcaggggttgcgacattttgacacctacgactactactacgttctggacgtctggggccaagggaccacggtcaccgtctcctca

SEQ ID NO: 11 - DNA, 363 bp, synthetic construct
  source 1..363: mol_type = other DNA
caggtgcagctggtgcagtctggggctgaggtgaagaagcctggggcctcagtgaaggtttcctgcaaggctgctggatacaccttcggtacctatggggtgcattgggtgcgccaggcccccggacaaacgcttgagtggatgggatggatcaacgctggcaatgataacacaaaatattcacagaagttccagggcagactcaccattaccagggacacatccgcgagcacagcctacatggacttgagcagcctgagatctgaagacacggctgtctattactgtgcgagagaaaaggacatgtactatggttcgccatgggacgtctggggccaagggaccacggtcaccgtctcctca

SEQ ID NO: 12 - DNA, 396 bp, synthetic construct
  source 1..396: mol_type = other DNA
caggtgcagctgcaggagtcggggccaggactgataaagtcttcacagaccctgtccctcacctgcactgtctctggtggctccatcagtagtggtggttattactggagctgggtccgccagcacccagggaagggcctggagtggattggaaacatcgatcacagcgggagaaccaactacaacccgtccctccagagtcgcattagcgtttcaacagacacgtctaagaatgagttctccctgaagttgacctctgtgactgccgcggacacggccgtgtatttctgtgccagagggggccggggttacgatcttttgattggttcttatagagaggactactactatggtctggatgtctggggccaagggaccacggtcaccgtctcctca

SEQ ID NO: 13 - DNA, 366 bp, synthetic construct
  source 1..366: mol_type = other DNA
caggtgcagctggtgcagtctggggctgaggtgaggaagcctggggcctcggtgaagatttcctgcaaggcatctggatactccttcaccagctcctatatacactgggtgcgacaggcccctggacaagggcttgagtggatgggaataatcaaccctaagggtactggcgcaaagtatgcacagaagttccagggcagagtcaccatgaccagcgacacgtccacgaatacagtccacatggagctgagcagcctgagatctggggacacggccgtctattactgtgcgagagcattaccaaattttgactacttatctgccctttattattggggccagggaaccctggtcaccgtctcctca

SEQ ID NO: 14 - DNA, 378 bp, synthetic construct
  source 1..378: mol_type = other DNA
caggtgcagctggtgcagtctggggctgaggtgaagaagcctgggtcctcggtgaaggtctcctgcaaggcctctggaggcaccctcaggctttctgaaataagttgggtgcgacaggcccccggacaagggcttgagtggatggcatatatcaatcctgtccttgggacagctatctacgcacagaagttccagggcagggtcacgatttctgcggacgattctacgactacagcctacctggagctgaacagcctgagatctgaagacacggccgtctattactgtgcgagagaggagattaaatattgtcgtggcagtagttgctatgggtggttcgacccctggggccagggaactctggtcaccgtctcctca

SEQ ID NO: 15 - DNA, 363 bp, synthetic construct
  source 1..363: mol_type = other DNA
caggtgcagctggtgcagtctggggctgaggtgaagaagcctggggcctcagtgaaggtttcctgcaaggcttctggatacatcttcactaactatgctttgcattgggtgcgccaggcccccggacaaaggcttgagtggatgggatggatcaacgctgacagtagtaacacaatatattcacagaagttccagggcagagtcaccattaccagggacacatccgcgagcacagcctacatggagctgagcagcctgagatctgaagacacggctgtgtattactgtgcgagagataaggtcatgtactatggttcgccatgggacgtctggggccaagggaccacggtcaccgtctcctca

SEQ ID NO: 16 - DNA, 372 bp, synthetic construct
  source 1..372: mol_type = other DNA
caggtgcagctaaaccagtggggcgcaggactgttgaagccttcggagaccctgtccctcacgtgcgctgtctatggtgacgccttcagcggtaactactggagctggatccgccagcccccagggaaggggctgcagtggattggcgaaattaatcatagtggaaatactaattacgacccgtccctcgagagtcgagtcagtatatccttagacacgtccaagaaccagttgtccctgaaactgaactctgtgaccgccgcggacacggctctatattattgtgcgcgaggccagaggaattgtagtggtggtaactgctactcaggcctctgggcgtggggccagggaaccctggtcaccgtctcctca

SEQ ID NO: 17 - DNA, 369 bp, synthetic construct
  source 1..369: mol_type = other DNA
caggtgcagctggtgcagtctgggccagaggtgaagaagcctgggtcctcggtaaaagtctcctgcaaggcttctggggacagcatcagcaactatggtatcagctgggtgcgacaggcccctggacaagggcttgagtggatggctgcgatcatccctgtctttggtaaaacaaaatatgcacagaagttccagggcagagtcacgattaccgcggacgaatccacgaccgcaggctacatggagctgaccaacctgagatttgaggacacggccgtctattactgtgcgatgaagctcctgccaccgcgactactccattacgggatggacgtctggggccaagggacctcggtcaccgtctcctca

SEQ ID NO: 18 - DNA, 381 bp, synthetic construct
  source 1..381: mol_type = other DNA
caggtgcagctgcagcagtggggcgcaggactgttgaagccttcggagaccctgtccctcacctgctctgtctatggaggatccgtcactggttactactggagctggatccgccagcccccagggaaggggctggagtggattggcgaagttcatcatagtggaagcaccaagtacaacccgtccctcaagagtcgagtcaccatctcagtagacacgtccaagagccagttctccctgaagctgagctctgtgaccgccgcggacacggctgtttattactgtgcgagagaggttgggttttacgatcttttgactgattccgcccgggtgggctactttgactactggggccagggatccctggtcaccgtctcctca

SEQ ID NO: 19 - DNA, 381 bp, synthetic construct
  source 1..381: mol_type = other DNA
caggtgcagctggtggagtctgggggaggcttggtacagcctggggggtccctgagactctcctgtgcagcctctggattcacctttagcagctatgccatgagctgggtccgccaggctccagggaaggggctggagtgggtctcaattattagcggtagtggtgatatcacatacaacgcagactccgtgaagggccggttctccatctccagagacaattccgaagacactctatatcttcaaatgaacagtttgagagccgaggacacggccgtatattactgtgcgaaatccccgggtatgattaggtttgcgggcgtcatcgttaaacctgtgtttgactcctggggccagggaaccctggtcaccgtctcctca

SEQ ID NO: 20 - DNA, 363 bp, synthetic construct
  source 1..363: mol_type = other DNA
caggtgcagctggtgcagtctggagctgaggcgaagaagcctggggcctcagtgaaagtctcctgcaaggctgctggttacagctttatcacctatggtatcaactgggtgcgacaggcccctggacaagggcttgagtggatgggatggatcagcgtttacagtggtaatacaaagtttgcacagaaattccagggcagagtcaccatgaccagagatacatccacgagcacagcctacatggagctgaggagcctgagatctgacgacacggccgtgtattactgtgcgagagcgggggaagtctacggtgactattactttgactactggggccggggaaccctggtcaccgtctcctca

SEQ ID NO: 21 - DNA, 363 bp, synthetic construct
  source 1..363: mol_type = other DNA
caggtgcagctggtgcagtctggggctgaggtgaagaagcctggggcctcagtgaaggtttcctgcaaggcttctggatacaccttcacttcctatgctatccattgggtgcgccaggcccccggacaaaggcttgagtggatgggatgcatcaacgctggcaatgataacacaaaatcttcacagaagttccagggcagactcacctttagcagggacacatccgcgagtattgcctacatggagctgagcagcctgagatctgaagacacggctgtgtattactgtgcgaaggagcagtcgaattactatggttcgggggttgactactggggccagggaaccctggtcaccgtctcctca

SEQ ID NO: 22 - DNA, 369 bp, synthetic construct
  source 1..369: mol_type = other DNA
caggtgcagctggtgcagtctggggctgaggtgaagaagcctgggtcctcggtgaaggtctcctgcaaggcttctggagacaccttcagcggctattctattacctgggtgcgacaggcccctggacaagggcttgagtggatgggagggatcatccctctctttgacacagcaaactacgcacagaagttccagggcagagtcacgatgacggcggacgaatccacgaacacagcctacatggagctgagcagcctgagatctgaggacacggccgtctattactgtgcgagagatccttggatagcagtgggcggagtggggtactttgaaaactggggccagggaaccctggtcaccgtctcctca

SEQ ID NO: 23 - DNA, 366 bp, synthetic construct
  source 1..366: mol_type = other DNA
caggtgcagctggtggaatctgggggagacttggtccagcctggggggtccctgagactcgcctgtgcagcctctggattcacctttagcaactatgccatgaactgggtccgccaggctccagggaaggggctagagtggctcgcaggtattaatgacagtggcgatttcacatactattcagaccccgtgaaggcccgattcagcatctccagagacaattcgaagaacacgctttttctgcaaatgaacagcctgagagccgaggacacggccatatattactgtgcgaaagttgcccattcctattatcgaaaatcgcactttgacttctggggacagggaaccctggtcaccgtctcctca

SEQ ID NO: 24 - DNA, 375 bp, synthetic construct
  source 1..375: mol_type = other DNA
caggtgcagctggtggagtctggggggggcgtggtccagcctgggaggtccctgagactctcatgtgtagcctctggattcagcttcgatacctattctatgcactgggtccgccaggctccaggcaaggggctggagtgggtggcagttacatcatatgatggcaacgataaatactacgcagactccgtgaagggccgattcaccatctccagagacaatttcaagaacatgctaaatctgcaattgaacagcctgagaactgaggacacggctgtgtatttctgtgcgagagatttcatagtggggtctaccttccgaaagcgggcgctctttgactactggggccagggaacccaggtcaccgtctcctca

SEQ ID NO: 25 - DNA, 393 bp, synthetic construct
  source 1..393: mol_type = other DNA
caggtgcagctggtgcagtctggagctgaggtgaagaagcctggggcctcagtgaaggtctcctgtaaggctcttggttacacctttaccagttatggtctcagctgggtgcgacaggcccctggacaggggcttgagtggatgggatggatcagcgcatacaatggtaacacaaagtacgcacagaagttccagggcagagtcaccatgacctcagagacatccacgagtactgcctacatggaactgaggcgcctgagatctgacgacacggccgtgtattactgtgcgagagaaggggcagggactgtcgtgctgagagccaatgtgggacactcgtcccactacggaatggacgtctggggccaaggcaccacggtcaccgtctcctca

SEQ ID NO: 26 - DNA, 381 bp, synthetic construct
  source 1..381: mol_type = other DNA
caggtgcagctgcagcagtggggcgcaggactgttgaagccttcggagaccctgtccctcacctgcgctgtccatggtcggtccttcattggttactactggagctggatccgccagcccccagggaaggggctggagtggattggggaagtcaatcatagtgggagctccaattacaacccgtccctcaagagtcgagtcaccatatcagcagagacgtccgaaaaccagttctccctgaagctgacctctgtgaccgccgcggacacggctgtctattactgtgcgagagcgatagggtattacgatattttgactgattcttataacaccttccacttgactccctggggccagggaaccctggtcaccgtctcctca

SEQ ID NO: 27 - DNA, 381 bp, synthetic construct
  source 1..381: mol_type = other DNA
caggtgcagctggtgcagtctggggctgaggtgaagaagcctgggtcctcggtgaaaatctcctgcaaggcttctggagtcagcttcagcaactttgctgtcagttgggtgcgacaggcccctggacaagggctggagtggatgggagggatcatccctttcttgggaaccacaaagtacgcacagaagatccagggcagagtcacgattaccgcggacgaatccacgaccacagcctacatggagctgagcagcctgagatctgaggacacggccgtatattactgtgcgagcgggttttgtagtggtggtagttgttacgtgaatttcatttacggtatggacgtctggggccaagggaccacggtcaccgtctcctca

SEQ ID NO: 28 - DNA, 384 bp, synthetic construct
  source 1..384: mol_type = other DNA
caggtgcagctggtggagtctgggggaggtgtggtacggccgggggggtccctgagactctcctgtgcagcctctggattcagctatgatgactatgacatgaactgggtccgccaagctccagggaaggggctggagtgggtctctggtattacttggactggaagtaatagtggttacgcagactctgtgaagggccgattcatcatctccagagacaacgccaagaagaccctatatctacaaatgaacagtctgggagtcgaagacacggccttgtactactgtgtgagggggtcgtattgtactagtaccagttgcgcgctctacgactatagaggtttggacgtctggggccaagggaccgcggtcaccgtctcctca

SEQ ID NO: 29 - DNA, 408 bp, synthetic construct
  source 1..408: mol_type = other DNA
caggtgcagctggtggagtcggggggaggcttggtacagccgggggggtccctgagactctcctgtgcagcctctggattcagctttagcaactatgccatgagctgggtccgccaggctccagggaaggggctggagtgggtctcaggtttccgtggtagtgatggtagcacatactacgcagactccgtgaagggccggttcaccatctccagagacaattccaagaacacactgtttctgcaaatgcacagcctgagagccgacgacacggccgtatattactgtgcgaaagatcagatcggccatacacctccccaacccggactagggatcggggagagcgaggactacttctactacgctatggacgtctggggccaagggaccacggtcaccgtctcctca

SEQ ID NO: 30 - DNA, 369 bp, synthetic construct
  source 1..369: mol_type = other DNA
caggtgcagctggtggagtcggggggaggcgtggtccagcctgggcggtccctgagactctcctgtgaagtctctggaatccccttcagtagttatggcatgcactgggtccgccaggctccaggcaaggggctggagtgggtgggtgttgtgtcatatgatggcacgaaaacatcctatggagactccgtgaagggccgattcaccatctccagagacaattccaagaacacgctgtatctgcaaatgaacagcctgaaaaatgaggacacggctgtgtatttttgtgcgaaagataaagcactcgtggtcgctcctaccccctacttctacttctacggtaaagggaccacggtcaccgtctcctca

SEQ ID NO: 31 - DNA, 357 bp, synthetic construct
  source 1..357: mol_type = other DNA
caggtgcagctggtgcagtctggacctgaggtgaagaggcctggggcctcagtgaaggtctcctgcaagacctccggttacatctttactagtcatggtatcagctgggtgcgacaggcccctggacaagggcttgagtggatgggatggatcagcccttacagtggtaacacaaagactgtacagaagctccagggcagagtcaccctgaccgcagacacatccacgaaaacagcctacatggagctgaggagcctgacatcagacgacacggccgtctattattgtgcaagagcggatgggaactactacgtcatggacgtctggggccaagggaccacggtcaccgtctcctca

SEQ ID NO: 32 - DNA, 373 bp, synthetic construct
  source 1..373: mol_type = other DNA
caggtgcagctgcaggagtcggacccaggactggtgaaaccttcacagaccctgtccctcacctgcactgtctctggtggctccatcaacagtaatgattactactggagttggatccgccagcccccagggaagggcctggagtggattgggtacatctcttacagtgggaccacctactacaacccgtccctcaagagtcgaatttccatatcagttgacacgtccaagaaccagttctccctgaatctgagctctgtgactgccgcagacacggccgtatattactgtgccagagagagggggggctcctggttatccgtggatgcagctattattactggggcgtttgactactgtcaccgtctcctca

SEQ ID NO: 33 - DNA, 387 bp, synthetic construct
  source 1..387: mol_type = other DNA
caggtgcagctacagcagtggggcgcaggactgttgaagccttcggagagcctgtccctcacctgcgtagtctatggtgggtccttcagtggttactactggagctggatccgccagcccccagggaaggggctggagtggattggggaaatcaatcctactggaagcaccaactacaatccgtccctcaacagtcgagtcaccatatcagtagacaggtccaagaaccacttgtccctgaagctgagctctgtcaccgccgcggacacggctgtgtatttctgtgcgagatttctcccccccgactacgatcttttgactgattattttgaccccccgggtgcttttgatatctggggccaagggacaatggtcaccgtctcctca

SEQ ID NO: 34 - DNA, 369 bp, synthetic construct
  source 1..369: mol_type = other DNA
caggtgcagctggtgcagtctggggctgaggtgaagaagcctgggtcctcggtgaaggtctcctgcaaggcttctggaggctccttcagcaatgatcctattagttggatccgacaggcccctggacaagggcttgagtggatgggagggatcatccctaaacttggtaaatcaacttacgctcagaagttccagggcagagtcacgattaccgcggacgaagccgcgagcacaggttacatggagctgagcagcctgagatctgaggacacggccgtgtattactgtgcgagaggaggccgcgtattgcgacattttgactggctagtgtacttctggggcaaagggaccacggtcaccgtctcctca

SEQ ID NO: 35 - DNA, 366 bp, synthetic construct
  source 1..366: mol_type = other DNA
caggtgcagctacagcagtggggcgcaggactgttgaagccttcggagaccctgtccctcagctgcgctgtctatggtgggtccttcagtggttactactggagttggatccgccagtccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtctattactgtgcgagagatacgggatattacgatattttgactgattatttttcaagggggggcaaagggaccacggtcaccgtctcctca

SEQ ID NO: 36 - DNA, 375 bp, synthetic construct
  source 1..375: mol_type = other DNA
caggtgcagctggtgcagtctggggctgaggtgaagaagcctgggtcctcggtgaaggtctcctgcaaagcttctggaggctccttcagtaactatggtatccactgggtgcgacaggcccctggactagggcttgagtggatgggagggatcatccctaccttacgtacaccaaactacgcacagaaattccagggcagagccacgattaccgcggacgaatcctcgaacacatcctacatggagctgagcagcctgagatctgaagacacggccgtgtattactgtgcgagggatccgggtatagtatcaactggtatttactacaccggtatggacgtctggggccaagggaccacggtcaccgtctcctca

SEQ ID NO: 37 - DNA, 363 bp, synthetic construct
  source 1..363: mol_type = other DNA
caggtgcagctggtgcagtctggagctgaggttcagaagcctggggcctcagtgaaggtctcctgcaaggcttctggttatttttttaccagccatggtatcgtctgggtgcgccaggcccctggacaagggcttgagtggatgggatggatcagcccttacagtggcaacacaaagtatgcacagaaactccagggcagagtcaccatgaccacagacacatccacgagcacagcctacatggagctgaggagcctgagatctgacgacacggccgtgtatttctgtgcgagagcgggagaagtctacggtgactactacttcgactactggggccagggaaccctggtcaccgtctcctca

SEQ ID NO: 38 - DNA, 366 bp, synthetic construct
  source 1..366: mol_type = other DNA
caggtgcagctggtacagtctggggcagaggtgaagaagcctgggacctcagtgaaggtttcctgcaaggcatctggatacaccttcaccagtgtctatatgcactgggtgcgacagggccctggacaagggcttgagtggatgggaataatcaaccctagagatggtagcacaaactacgcacagaaattccagggcagagtcaccatgaccagggacacgtccacgagcacagtctacatggagctgagcaccctgagatccgacgacacggccgtgtattactgtgcgcgaacattaccatcttttgactggttatctgccctttattactggggccagggaaccccggtcaccgtctcctca

SEQ ID NO: 39 - DNA, 348 bp, synthetic construct
  source 1..348: mol_type = other DNA
caggtgcagctggtgcagtctggggctgaggtgaagaagcctggggcctcagtgaaggtttcctgcaaggcatctggatacaccttcaccagctactatatgtactgggtgcgacaggcccctggacaagggcttgagtggatgggaataatcaaccctagtggtgatagcacaagctacgcacagaagttccagggcagagtcaccatgaccagggacacgtccacgagcacagtctacatggagctgagcagcctgagatctgaggacacggccatgtattactgtgtctactactactacggtatggacgtctggggcaaagggaccacggtcaccgtctcctca

SEQ ID NO: 40 - DNA, 357 bp, synthetic construct
  source 1..357: mol_type = other DNA
caggtgcagctggtggagtctgggggaggcttagctcagccgggggggtccctgagactctcctgtgcagcctctggattcgccttcagtagctcctggatacactgggtccgccaagctccggggaaggggctggtgtggctctcacgtgttaattatgatgggattagcacaacttacgcggactccgtgaagggccgattcaccatctccagagacaacgccaagagcacggtgtttctgcaaatgaacagtctgagaggcgaggacacggctgtgtattactgtgcaacatcgtacttatctcgtggccccatgggcgactggggccagggaaccctggtcaccgtctcctca

SEQ ID NO: 41 - DNA, 396 bp, synthetic construct
  source 1..396: mol_type = other DNA
caggtgcagctggtgcagtctggggctgaggtgaagaagcctggggcctcagtgaaggtttcatgcaaggcatctggatacaccttcaccagttactatatgcactgggtgcgacaggcccctggacaagggcttgagtggatgggaataatcaaccctagtggtggtagcacaacctacgcacagaagttccagggcagactcaccatgagcagggacacgtccacgagcacagtcaacatggagctgagcagcctgagatctgaggacacggccgtgtattactgtgcgagaggggtcgggcacgggtgggtcggggaggtattatatcgggatgctgataaccactacggtatggacgtctggggccaagggaccacggtcaccgtctcctca

SEQ ID NO: 42 - DNA, 369 bp, synthetic construct
  source 1..369: mol_type = other DNA
caggtgcagctggtgcagtctggggcagaggtgaagaagactgggtcttcggtgaaggtctcctgcaaggcttctggaggccccgtcaacagctatggtataagttgggtgcgacaggcccctggacaagggcttgaatggatgggagcaatcatccctgtttttggtagagtaaaatacgcacagaagttccagggcagagtcacgattaccgcggacgagtccacgaccacagccaacatggaagtgaccagcctgagatttgaggacacggccgtctattactgtgcgatgaagttagaagtcccgcgacttttgcattatggtatggacgtctggggccaaggaaccacggtcaccgtctcctca

SEQ ID NO: 43 - DNA, 381 bp, synthetic construct
  source 1..381: mol_type = other DNA
caggtgcagctacagcagtggggcgcaggactgttgaagccttcggagaccctgtccctcacctgcgctgtccatggtcggtccttcattggttactactggagctggatccgccagcccccagggaaggggctggagtggattggggaagtcaatcatagtgggagctccaattacaacccgtccctcaagagtcgagtcaccatatcagcagagacgtccgaaaaccagttctccctgaagctgacctctgtgaccgccgcggacacggctgtctattactgtgcgagagcgatagggtattacgatattttgactgattcttataacaccttccactttgactcctggggccagggaaccctggtcaccgtctcctca

SEQ ID NO: 44 - DNA, 381 bp, synthetic construct
  source 1..381: mol_type = other DNA
caggtgcagctacagcagtggggcgcaggactattgaagccttcggagaccctgtccctcacctgcgctgtctatggtgggtccttcagtggttactactggaactggatccgccagtccccagggaaggggctggagtggattggggaaatcaatcatagtgggagcaccaactacaacccgtcactcaagagtcgagtcaccatgtcagtagacacgtccaagaaccagttctccctgaagctgagctctgtgaccgccgcggacacggctgtgtattactgtgcgagaggcctggggtattacgatactgtgactggttatatcgggtcgggaggtatggacgtctggggccaagggaccacggtcaccgtctcctca

SEQ ID NO: 45 - DNA, 375 bp, synthetic construct
  source 1..375: mol_type = other DNA
caggtgcagctggtgcaatctggggctgaaattaagaagcctggggcctcagtgaaggtttcctgcaaggcatctggatacaccttcatcagtaactatttccactgggtgcgacaggcccctggacaagggcttgagtggatgggaataatcaaccctagttctggtagtacaacctatggacagaaattccagggcagagtcaccctgactagcgacacgtccgcgagcatagtctacctggagctgagccgcctgagatctgaagacacggccgtgtattattgtgttagtccccggatagcacattgtcgtggtggtcggtgctacgagactctcgactggggccagggaaccctggtcaccgtctcctca

SEQ ID NO: 46 - DNA, 369 bp, synthetic construct
  source 1..369: mol_type = other DNA
caggtgcagctggtgcagtctggggcagaggtgaagaagcctgggtcctcggtgagggtctcctgcaaggcttctggaggcaccttcagcagctatggtatcagctgggtgcgacaggcccctggacaagggcttgagtggctgggagggatcatccctacgtttggtagagcaaaatacgcacagaagttccagggcagagtcacgattaccgcggacgaatctacgtccgcttccaacatggaggtgaccagtctgagatttgaggacacggccgtgtattactgtgcgataaagattctaccaccacgactgctcttttacggaatggacgtctggggcccagggaccacggtcaccgtctcctca

SEQ ID NO: 47 - DNA, 387 bp, synthetic construct
  source 1..387: mol_type = other DNA
caggtgcagctgcagcagtggggcgcaggactgttgaagccttcggagaccctgtccctcacctgcgctgtctatggtgcgcccttcagtggttactactggaactggatccgccagcccccagggaaggggctggagtggattggggaactcaatcatagtggaggcaccaactacaacccgtccctcaagagtcgaatcaccatgtctgtagacccgtccaacaaccagttttccctgaggttgagctctgtgaccgccgcggacacggctgtgtatttctgtgcgaggtccctgccggccgattacgatcttttgactggttctaatgatttattcggtgcttttgatctctggggccaagggaccgtggtcaccgtctcctca

SEQ ID NO: 48 - DNA, 381 bp, synthetic construct
  source 1..381: mol_type = other DNA
caggtgcagctggtggagtcggggggaggcttggtacagccgggggggtccctgagactctcatgtgcaggctctggattctacttcagtgcctatagcatgaactgggtccgccaggctccagggaaggggctggagtgggtttcatacattagtagtagtggtagaaccatttactacgcagactctgtgaggggccgatttaccatctccagagacaatgccaagaattcactgtatctgcgaatggacagcctgagagtcgaggacacggctatctattactgtgcgagagatcaggggttgcgacattttgacacctacgactactactacgttatggacgtctggggccaagggaccacggtcaccgtctcctca

SEQ ID NO: 49 - DNA, 381 bp, synthetic construct
  source 1..381: mol_type = other DNA
caggtgcagctggtggagtctgggggaaatatggtacggcctggggggtccctgagactctcctgtgcagcctctggattctcgtttgatgactatgggatgagttgggtccgccacgttccagggaagggactggagtgggtcgctggtattaattggaagggtgatagtataaattatgcagactctgtgaggggccgattcactatctccagagacaacgccaagaactccctgtatctgcaaatgaacagtctgagagccgaggatacggccttgtatcactgtgcgagaggagacattactttgggtcggggagtttattactcttatcacggtttggacgtctggggccaagggaccacggtcaccgtctcctca

SEQ ID NO: 50 - DNA, 369 bp, synthetic construct
  source 1..369: mol_type = other DNA
caggtgcagctgcaggagtccggctcacgactggtaaacccttcagagaccctgtccctcacctgcgaagtctctggtggctccatcagtagcggtgattactcctggacctggatccggcagccgccagggaaggacctggagtggattgggtacatctaccatagtgggagcacctatttcaacccgtccctcaagagtcgagtcagcatgtcagtagacagttccaagaaccagctctccctgcacctgaggtctgtgaccgccgcggacacggccgtatattactgtgccagactcagtaacatgtggcccaagtacaaccacatcgactcctggggccagggaaccctggtcaccgtctcctca

SEQ ID NO: 51 - DNA, 375 bp, synthetic construct
  source 1..375: mol_type = other DNA
caggtgcagctggtgcagtctggggctgaaatacagaagcctggggcctcagtgaaggtttcctgcagggcatctggatacacttacatcagcacctctttccactgggtgcgacaggcccctggacaggggcttgagtggatgggagtaatcaagactgatgatggtagtaccagcgacacacagaagttccagggcagactcaggatgacctgggacacgtccacgagcacagtttacatggaactgagcagcctgagatctgacgacacggccgtgtattattgtgttagtccccgcctaggacattgtcgtcgtggccggtgctacgagactctcgactggggccagggatccctggtcaccgtctcctca

SEQ ID NO: 52 - DNA, 429 bp, synthetic construct
  source 1..429: mol_type = other DNA
caggtgcagctggtggagtctgggggaggcttggtacagcctggggggtccctgagactctcctgtgaagcctctggattcaccttcagtaactataacatgaactgggtccgccaggctccagggaaggggctggagtgggtttcatatattagtagtagtagtagtaccatatattacacagactctgtgaagggccgattcaccatctccagagacaatgccaagaactcactgtatctgcaaatgagcaggctgagagccgaggacacggctgtgtattactgtgcgagagcggacgatcccacgacaattctagtccccggaaatatcgcagcacctggactcgaaacagagttagggttggatgaccaccagtaccgcggtatggacgtctggggccaagggaccacggtcaccgtctcctca

SEQ ID NO: 53 - DNA, 357 bp, synthetic construct
  source 1..357: mol_type = other DNA
caggtgcagctggtgcagtctggacctgaggtgaagaagcctggggcctcagtgaaggtctcctgcaaggcttctggttacatctttaccagtcatggtatcacctgggtgcgacaggcccctggccaagggcttgagtggatgggatggatcagcgcttacagtggtaacacaaactatccacagaaggtccaggacagagtcaccatgaccagagacacatccacgagcacagcctacatggagttgaggagcctgagacctgacgacacggccgtatattattgtgtgagaagtggaaactacccgttcatcttcgacccctggggccagggaaccctggtcaccgtctcctca

SEQ ID NO: 54 - DNA, 381 bp, synthetic construct
  source 1..381: mol_type = other DNA
caggtgcagctggtggagtctgggggaggcttggtacagccgggggggtccctgagactctcctgtgcagcctctggattcgcctttagcacctatgtcatgagctgggtccgccaggctccagggaagggtctggagcggatctcgtctattagtggtcgtggaacaacgtacgcagactccgtgaagggccgattcaccgtctccagagacaattccaagaacacgttgtatctgcaaatgaacagtctgagagccgaggacacggccgtatattactgtgcaaaagagggcgccgagatacgacttttggactggccgatattgatagactggtacttcgatctctggggccgtggcaccctggtcaccgtctcctca

SEQ ID NO: 55 - DNA, 354 bp, synthetic construct
  source 1..354: mol_type = other DNA
caggtgcagctggtgcagtctggagctgaggtggtgaagcctggggcctcagtgagagtctcctgcaaggcttctggttacatctttaccagccatgggatcgcctgggtgcgacaggcccctggacaagggcttgagtcgatgggatggatcagcgcttacagtggaaacacaaactatgcacagaaattccagggcagagtcacaattaccagagacacatccacgagcacagcctatttggagttgaggagcctccgatctgacgacacggccgtatactactgtgcgagagtggagcaattgctactaggggacttctggggccagggaaccctggtcaccgtctcctca

SEQ ID NO: 56 - DNA, 369 bp, synthetic construct
  source 1..369: mol_type = other DNA
caggtgcagctggtgcagtctggggcagaggtgaagaagactgggtcttcggtgaaggtctcctgcaaggcttctggaggccccgtcagcagctatggtataagttgggtgcgacaggcccctggacaagggcttgaatggatgggagcaatcatccctgtttttggtagagtaaaatacgcacagaagttccagggcagagtcacgattaccgcggacgagtccacgaccacagccaacatggaagtgaccagcctgagatttgaggacacggccgtctattactgtgcgatgaagttagaagtcccgcgacttttgcattatggtatggacgtctggggccaaggaaccgcggtcaccgtctcctca

SEQ ID NO: 57 - DNA, 369 bp, synthetic construct
  source 1..369: mol_type = other DNA
caggtgcagctggtgcagtctggggcagaggtgaagaagtctgggtcctcggtgaaggtctcctgcaaggcttctggagacaccttcagtagttatggtatcacctgggtgcgccaggcccctggacaagggcttgagtggatgggaggtatcatccctgtgtttggaagagcaaaatacgcacagaagttccagggccgagtcaccattaccgcggacgaatccacgaccacaggctacatggaggtgaccagactgagatttgaagacacggccgtatattattgtgcgataaaggtcgaagcaccgcgactactcttttacggtatggacgtctggggccaagggaccacggtcaccgtctcctca

SEQ ID NO: 58 - DNA, 357 bp, synthetic construct
  source 1..357: mol_type = other DNA
caggtgcagctggtgcagtctggagcagaggtgaaaaagcccggggagtctctgaagatctcctgtaagggttctggatacagtttcaccagttactgggtcggctgggtgcgccagatgcccggaaaaggcctggagtggatggggatcatccatcctgatgtgtctgaaattagatacagcccgtccctccaaggccacgtcaccatgtcagtcgacaagtccatcagtaccgcctacctgcagtggagcagcctgaaggcctcggacaccgccatttattactgtgcgagacatgggtatgggtcgggcttctttgacaactggggccagggaaccctggtcaccgtctcctca

SEQ ID NO: 59 - DNA, 340 bp, synthetic construct
  source 1..340: mol_type = other DNA
gatgttgtgatgactcagtctccactctccctgcccgtcacccttggacagccggcctccatctcctgcaggtctggtcaaagcctcgtacacagtgatggaaacacctacttgaattggtatcaacagaggccaggccaatctccaaggcgcctaatttataaggtatctaaccgggaccctggggtcccagacagattcagcggcagtgggtcaggcactgatttcacactgaaaatcagcagggtggaggctgatgatattggagtttattactgcatgcaaggtacatactggcctgagatcaccttcggccaagggacacgactggacattaaac

SEQ ID NO: 60 - DNA, 337 bp, synthetic construct
  source 1..337: mol_type = other DNA
gatattgtgatgactcagtctccactctccctgcccgtcacccctggagagccggcctccatctcctgcaggtctagtcagagcctcctgcatagtaatggatacaactatttggattggtatctgcagaagccagggcagtctccacagctcctgatctatttgggttctaatcgggcctccggggtccctgacaggttcagtggcagtggatcaggcacagattttacactgaaaatcagcagagtggaggctgaagatgttgggatttattactgcatgcagggtctacaaacctcgttcactttcggcggagggaccaaggtggagatcaaac

SEQ ID NO: 61 - DNA, 334 bp, synthetic construct
  source 1..334: mol_type = other DNA
gatattgttatgacccagtctccattctccctgcccgtcacccctggagagccggcctccatctcctgcaggtcaagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgcggatgttgggatttattactgcatgcaggctctacaaactccgacgttcggccaagggaccaaggtggaaatcaaac

SEQ ID NO: 62 - DNA, 337 bp, synthetic construct
  source 1..337: mol_type = other DNA
gatattgtgatgactcagtctccactctccctgcccgtcacccctggagagccggcctccatctcctgcaggtctagtcggagcctcctgcattttaatggatacgactatttggattggtacctgcagaagccagggcagtctccacagctcctgatctatttgggttctaatcgggcctctggggtccctgacaggttcagtggcagtggatcaggcacagattttacactgaaaatcagcagagtggaggctgaagatgttggggtttattactgcatgcaagctctacaaactcctcgcacgttcggccaagggaccaaggtggaaatcaaac

SEQ ID NO: 63 - DNA, 337 bp, synthetic construct
  source 1..337: mol_type = other DNA
gatattgtgatgactcagtctccagtctccctgcccgtcacccctggagagccggcctccatctcctgcaggtctagtcagagcctcctgcatagtaatggatacaactatttggattggtacctgcagaagccagggcagtctccacagctcctgatctatttgggttcttatcgggcctccggggtccctgacaggttcagtggcagtggatcaggcacagattttacactgagaatcagcagagtggaggctgaggatgttggggtttattactgcatgcaagctctacaaactcctcggacttttggccaggggaccaagctggagatcaaac

SEQ ID NO: 64 - DNA, 337 bp, synthetic construct
  source 1..337: mol_type = other DNA
gatattgtgatgactcagtctccactctccctgcccgtcacccctggagagccggcctccatctcctgcaggtctagtcagagcctcctgcacagtaatggatacaactatttggattggtacctgcagaagccagggcagtctccacagctcctgatctatttgggttcttatcgggccgccggggtccctgacagattcagtggcagtggatcaggcacagattttacactgaaaatcagcagagtggaggctgaggatgttggggtttattactgcatgcaagctctacaaactccgtggacgttcggccaagggaccaaggtggaaatcaaac

SEQ ID NO: 65 - DNA, 337 bp, synthetic construct
  source 1..337: mol_type = other DNA
gatattgtgatgactcagtctccactctccctgcccgtcacccctggagagccggcctccatctcctgcaggtctagccagagcctcctgcacagtaatggatacaactatttggattggtacctgcagaagccagggcagtctccacagctcctgatctatttgggttcgtatcgggccgccggggtccctgacagattcagtggcagtggatcaggcactgattttacactgcaaatcagcagagtggaggctgaggatgttggcctttattactgcatgcaagctctacaaactccgtggacgttcggccaagggaccaaggtggatatcaaac

SEQ ID NO: 66 - DNA, 322 bp, synthetic construct
  source 1..322: mol_type = other DNA
gacatccatatgacccagtctccattctccctgtctgcatctgtaggagacagagtcaccatcacttgccgggccagtcagagtattagtggctggttggcctggtatcagcagaaagtagggaacgcccctaagctcctgatctataaggcgtctagtttagaaagtggggtcccatcaagattcagcggcagtggatctgggacagaattcactctcagcatcagcagcctgcagcctgatgattttgcaacttattactgccaacagtataatagttacccattcactttcggccctgggaccaaagtggatatgaaac

SEQ ID NO: 67 - DNA, 322 bp, synthetic construct
  source 1..322: mol_type = other DNA
gacatccatatgacccagtctccattctccctgtctgcaactgttggagacagagtcaccatctcttgccgggccagtcagagtattggtggctggttggcctggtatcagcagaaaccagggaaagcccctaagctcctgatctatcaggcgtctaccttagaaactggggtcccatcaaggttcagcggcagtggatctgggacagaattcactctcaccatcagcagcctgcagcctgatgattttgcaacttattactgccaacactataatagttatttattcactttcggccctgggaccaaagtggatatcaaac

SEQ ID NO: 68 - DNA, 322 bp, synthetic construct
  source 1..322: mol_type = other DNA
gacatccagatgacccagtctccattctccctgtctgcatctgtaggagacagagtcaccatcacttgccgggccagtcagagtattagtagctggttggcctggtatcagcagaaaccagggaaagcccctaagctcctgatctataaggcatctagtttacaaagtggggtcccatcaaggttcagcggcagtggatctgggacagaattcactctcaccatcagcagcctgcagcctgatgattttgcaacttattactgccagcagtatcatactgattggtacacttttggccaggggaccaaggtggagatcaaac

SEQ ID NO: 69 - DNA, 322 bp, synthetic construct
  source 1..322: mol_type = other DNA
gacatccagatgacccagtctccttccaccctgtctgcatctgtaggagacagagtcaccatcacttgccgggccagtcagagtattagtaactggttggcctggtatcagcagaaaccagggaaagcccctaaactcctgatctataaggcatctagtttagaaagtggggtcccatcaaggttcagcggcagtggatctgggacagaattcactctcaccatcaacagcctgcagcctgatgattttgcaacttatcactgccaacaatacaaaagttattggaacagttttggccaggggaccaagctggagatgaaac

SEQ ID NO: 70 - DNA, 322 bp, synthetic construct; identical to SEQ ID NO: 69
  source 1..322: mol_type = other DNA

SEQ ID NO: 71 - DNA, 322 bp, synthetic construct
  source 1..322: mol_type = other DNA
gacatccagatgacccagtctcctttctccctgtctgcatctgtaggagacagagtcaccatcacttgccgggccagtcagagtattagtaactggttggcctggtatcagcagaaaccagggaaagcccctaaactcctgatctataaggcatctagtttagaaagtggggtcccatcaaggttcagcggcagtggatctgggacagaattcactctcaccatcaacagcctgcagcctgatgattttgcaacttatcactgccaacaatacaaaagttattggaacagttttggccaggggaccaagctggagatgaaac

SEQ ID NO: 72 - DNA, 325 bp, synthetic construct
  source 1..325: mol_type = other DNA
gacatccatatgacccagtctccagtctccctgtctgcatctgtaggagacagagtcaccatctcttgccgggcgagtcaggacattagtacttatcttgcctggtatcagcagagaccaggggaagttcctaagctcctgatctatgctgcatccactttgcaatcaggggtcccctctcggttcagtggcagtggatctgggacagatttcactctcaccatcagcagcctgcagcctgaagatgttgcaacttattactgtcaaaagtataacagtgcctcctcgatcaccttcggccaagggacacgactggatattaaac

SEQ ID NO: 73 - DNA, 321 bp, synthetic construct
  source 1..321: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaaactcctgatctatgctgcatccaatttacaaagtggggtcccatcaaggttcaacggcagtggatctggcactgatttcactctcaccatcaacagtctgcacctgaagattttgcaacttattactgtgtacaagactacaatttccctcgcacttttggccaggggaccaagctggagatcaaac

SEQ ID NO: 74 - DNA, 322 bp, synthetic construct
  source 1..322: mol_type = other DNA
gccatccatatgacccagtctccatcctccctgtctgcatctgtaggagacagagtcaccatcacttgccgggcaagtcagggcattagaaatgatttagcctggtatcagcataaaccagggacagcccctaaggtcctgatttatgctgcttccagtctacaagatggggtctcatcaaggttcagcggcagtggatctggcacattattcactctcaccatcagcagcctgcagcctgaagattttgcaacttattactgtctacaaggttacaattatcctcggacttttggccaggggaccaaggtggagatgaaac

SEQ ID NO: 75 - DNA, 322 bp, synthetic construct
  source 1..322: mol_type = other DNA
gccatccatatgacccagtctccattctccctgtctgcatctgtaggagacagagtcaccatcacttgccgggcaagtcagggcattagaaatgatttagcctggtatcagcataaaccagggacagcccctaaggtcctgatttatgctgcttccagtctacaagatggggtctcatcaaggttcagcggcagtggatctggcacattattcactctcaccatcagcagcctgcagcctgaagattttgcaacttattactgtctacaaggttacaattatcctcggacttttggccaggggaccaaggtggagatgaaac

SEQ ID NO: 76 - DNA, 322 bp, synthetic construct
  source 1..322: mol_type = other DNA
gccatccagatgacccagtctccattctccctgtctgcatctgtaggagacagagtcaccatcacttgccgggcaagtcagggcattagaaatgatttaggctggtatcagcagaaaccagggaaagcccctaagctcctgatctatgctgcatccagtttacaaagtggggtcccatcaaggttcagcggcagtggatctggcacagatttcactctcaccatcagcggcctgcagcctgaagattttgcaacttattactgtctacaagtttacaattatcctcggacgttcggccaagggaccaaggtggaaatcaaac

SEQ ID NO: 77 - DNA, 322 bp, synthetic construct
  source 1..322: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagctcctgatctatgctgcatccagtttacaaagtggggtcccatcaaggttcagcggcagtggatctggcacagatttcactctcaccatcagcggcctgcagcctgaagattttgcaacttattactgtctacaagtttacaattatcctcggacgttcggccaagggaccaaggtggaaatcaaac

SEQ ID NO: 78 - DNA, 322 bp, synthetic construct; identical to SEQ ID NO: 77
  source 1..322: mol_type = other DNA

SEQ ID NO: 79 - DNA, 322 bp, synthetic construct
  source 1..322: mol_type = other DNA
gacatccagatgacccagtctccattctccctgtctgcatctgtgggagacagagtcaccatcacttgccaggcgagtcaggacattaccaactatttaagttggtatcagcagaaaccagggacagcccctaaactcctgatctacgatgcatccaatttggaaacaggggtcccatcaaggttcagtggaagtggaactgggacacattttactttcaccatcagcagcctgcagcctgaagatattgcaacatattactgtcaacagtatgatactctcccgctcactttcggcggagggaccaaggtggagatcaaac

SEQ ID NO: 80 - DNA, 322 bp, synthetic construct
  source 1..322: mol_type = other DNA
gacatccatatgacccagtctccagtctccctgtctgcatctgtgggagacagagtcaccatcccttgccgggcaagccatcccattaacaactatttaaattggtatcagcagaaaccagggaaagcccctaacctcctgatctatgcttcatccaatttgcattctggggtcccatcgaggttcagtgccagtggatctggaacaaatttcactctcaccatcagcagtctacaacctgaagattttgcaatttacttctgtcaacagacttccagtacccctaacactttcggcggagggaccagggtcgacatcagac

SEQ ID NO: 81 - DNA, 322 bp, synthetic construct
  source 1..322: mol_type = other DNA
gacatccagatgacccagtctccatcctccctgtctgcatctgttggcgacagagtcatcatcacttgccgggcaagtgaaagcatcagtacctacttaggttggtatcagcagaaaccagggaaagcccctaaactcatcatctctgctgtttccactttgcaaagtggggtcccctcaaggttcagtggcagtggatctggaacagaattcactctcaccatcagcagtctgcaacctgaagattttgcaacttactactgtctacagagtcacagtatccctcaaacattcggccctgggaccacagtggatatcaagc

SEQ ID NO: 82 - DNA, 319 bp, synthetic construct
  source 1..319: mol_type = other DNA
gacatccagatgacccagtctccatcctccctgtctgcatctgtaggagacagagtcaccatcacttgccgggcaagtcagagcattggcagcaatttaaactggtatcaggcgaaaccagggaaagcccctaggctcctgatctattctgcatccactttgcaaagtggggtcccatcaacgttcagtggcagtggatctgggacagacttcactctcgccatcagcagtctgcaacctgaggattttgcaacttattattgtcaacagagttacagtgcccctagtttcggcggagggaccacggtggagatcaaac

SEQ ID NO: 83 - DNA, 325 bp, synthetic construct
  source 1..325: mol_type = other DNA
gacatccagatgacccagtctccattctccctgtctgcatctgtaggagacagagtcaccatcacttgccgggcaagtcagagtattagtaactatttaaattggtatcagcagaaatcagggaaagcccctaaaatcctgatctctgctgcatccactttgcaaagtggggtcccatcaaggttcagtggcagtggatctgggacagatttcactctcaccatcagcagtctgcaacctgaagattttgcaacttactactgtcaacacagtgacagtacccgtggtttcatttttggccgggggaccaagctggagatcaagc

SEQ ID NO: 84 - DNA, 322 bp, synthetic construct
  source 1..322: mol_type = other DNA
gacatccagatgacccagtctccattctccctgtctgcatctgtaggagacagagtcaccatcacttgccgggcaagtcagaccattagcagttatttaaattggtatcagcagaaaccagggaaagcccctaaactcctgatgtttgctgcatccactttacaaagtggagtcccatcaaggttcagtggcagtggatctgggacagatttcactctcaccatcagcggtctactacctgaagattttgcaacttactactgtcaacagagttacagtagcccgctcactttcggcggagggaccaaggtggagatcaaac

SEQ ID NO: 85 - DNA, 325 bp, synthetic construct
  source 1..325: mol_type = other DNA
gacatccagatgacccagtctccatcctccctgtctgcatctgtaggagacagagtcaccatcacttgccgggcaagtcagtacattagtgactatttaaattggtatcagcagaaaccagggaaagcccctaacctcctgatctatgctgcatccagtttgcaaagtggggtcccatcaaggttcagtggcagtggatctggcacagatttcactctcaccatcagcagtctgcaacctgaagattttgcaacttactactgtcaacagagttacggtacccctccgtggacgttcggccaagggaccaaggtggagttcaaac

SEQ ID NO: 86 - DNA, 322 bp, synthetic construct
  source 1..322: mol_type = other DNA
gacatccagatgacccagtctccatcctccctgtctgcatctgtaggagacagagtcaccatcacttgccgggcaagtcagagcattagcatctttttaaattggtatcggcagaaaccagggaaagcccctgagctcctgatctatgctgcatccagtttgcaaagtggggtcccatcaaggttcagtggcagtggatctgggacacatttcactctcaccatcagcagtctgcaacctgaagattttgcatcttactactgtcatcagagttacagtacccctcagacgttcggccaagggaccaaggtggaaatcaaac

SEQ ID NO: 87 - DNA, 322 bp, synthetic construct
  source 1..322: mol_type = other DNA
gacatccagatgacccagtctccattctccctgtctgcatctgtaggagacagagtcaccatcaattgccgggcaagtcagaccattttcagctatttgaattggtatcaacagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcaacagagttacagtccccctaatacgttcggccaagggaccaaggtggaaatcaaac

SEQ ID NO: 88 - DNA, 322 bp, synthetic construct
  source 1..322: mol_type = other DNA
gacatccagatgacccagtctccatcctccctgtctgcatctgtaggagacagagtcaccatcacttgccgggcaagtctgaccattttcagctatttaaattggtatcagcagaaaccagggaaagcccctaagctcctgatctatggtgcatccactttgcacagtggggtcccatcaaggttcagtggcagtggatctgggacagatttcactctcaccatcagcagtctgcaacctgaagattttgcaacttactactgtcaacagagtttcactgcccctcgtacgttcggccaagggaccaaggtggaaatcaaac

SEQ ID NO: 89 - DNA, 322 bp, synthetic construct
  source 1..322: mol_type = other DNA
gacatccagatgacccagtctccatcctccctgtctgcatctgtaggagacagagtcaccatcacttgccgggcaagtcagaccattttcagctatttaaattggtatcagcagaatccagggaaagcccctaagctcctgatctattctgcatcccgtttgcaaagtggggtcccatcaaggttcagtggcagtggatctgggacagatttcactctcaccatcagcagtctgcaacctgaagattttgcaacttactactgtcaacagagttacagtacccctcgaacgttcggccaagggaccaaggtggaaatcaaac

SEQ ID NO: 90 - DNA, 322 bp, synthetic construct
  source 1..322: mol_type = other DNA
gacatccatatgacccagtctccagtctccctgtctgcatctgtaggagacagagtcaccatcacttgccgggcaagtcagagcattagcaggtatttaaattggtatcagcagaaaccagggaaagcccctaagctcctgatctatgccgcatccagtttgcaaagtggggtcccatcaaggttcagtggcagtggatctgggacagatttcactctcaccatcagcagtctgcaacctgaagattttgcaacttacttctgtcagcagagttacagtcccctgtggacgttcggccaagggaccaaggtggagatcaaac

SEQ ID NO: 91 - DNA, 322 bp, synthetic construct
  source 1..322: mol_type = other DNA
gacatccagatgacccagtctccattctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccctcttcactttcggccccgggaccaaagtggatatcaaag

SEQ ID NO: 92 - DNA, 322 bp, synthetic construct
  source 1..322: mol_type = other DNA
gacatccatatgacccagtctccattctccctgtctgcatctgtaggagacagagtcaccatcacttgccgggcaagtcagaacattaacaggtatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttattactgtcaacagagttacagtacccctcacacttttggccaggggaccaagctggagatcaaac

SEQ ID NO: 93 - DNA, 340 bp, synthetic construct
  source 1..340: mol_type = other DNA
gacatcgtgatgacccagtctccagactccctggctgtgtctctgggcgagcgggccaccatcaactgcaagtccagccagagtgttttatacaactccaacaataagtacttcttaggttggtaccagcagaaaccgggacagcctccaaagctgctcatttcctgggcatctacccgggaatccggggtccctgaccgattcagtggcagcgggtctgggacagatttcactctcaccatcagcagcctgcaggctgaagatgtggcagtttattattgtcagcaatattatactactcccctcactttcggcggagggaccagggtggagatcaaac

SEQ ID NO: 94 - DNA, 331 bp, synthetic construct
  source 1..331: mol_type = other DNA
gaaatagtgatgacgcagtctccagccaccctgtctgtgtctccaggggaaagagccaccctctcctgcagggccagtcagagtattagcagccacttagcctggtaccagcagaaacctggccaggctcccagactcctcatctatggtgcatccaccagggccactggtgtcccagccaggatcagtggcagtgggtcggggacagacttcactctcaccatcagcagcctgcagtctgaagattttgcaatttattactgtcagcactatactaactggcctccgaggagattcactttcggccctgggaccaaactggatatcaaaa

SEQ ID NO: 95 - DNA, 325 bp, synthetic construct
  source 1..325: mol_type = other DNA
gaaattgttatgacccagtctccattctccctgtctttgtccccaggggaaagagccaccctctcctgcagggccagtcagagtgttagcaggtacttagcctggtaccaacagaagcctggccaggctcccaggctcctcatctatgatgcaagcaacagggccactggcgtcccagccaggttcagtggcagtgggtctgggacagacttcactctcaccatcagcagcctagagcctgaagattttgctctttattactgtcaccagagtaatcactggcctccgttcacttttggccaggggacgaagctggagatcaaac

SEQ ID NO: 96 - DNA, 319 bp, synthetic construct
  source 1..319: mol_type = other DNA
gaaattgtgttgacacagtctccagccaccctgtctttgtctccaggggaaagagccaccctctcctgcagggccagtcagagtgttagcatctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagatttcgcagtttattactgtcagcagcgtagcaactggccctctttcggcggagggaccaaggtggagatgaaac

SEQ ID NO: 97 - DNA, 325 bp, synthetic construct
  source 1..325: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcacctggcctccgctcactttcggcggagggaccaaggtggagatcaaac

SEQ ID NO: 98 - DNA, 325 bp, synthetic construct
  source 1..325: mol_type = other DNA
gaaattgtgttgacgcagtctccaggcaccctgtctttgtctccaggggaaagagccaccctctcctgcaggtctagtcagagcgtcctgcatagatacgtagcctggtaccagcatagacctgtgcaggctcccaggctcctcatctatgctgcgtccaatagggccactggcatcccagacaggttcactggaggggggtctgggacagacttcactctcaccatcagcagactggagcctgaagattttgcagtgttttactgtcagcagtatggtagctcacctcccactttcggcggagggaccaaggtggagatcaaac

SEQ ID NO: 99 - DNA, 325 bp, synthetic construct
  source 1..325: mol_type = other DNA
gaaattgtgttgacgcagtctccaggcaccctgtctttgtctccaggggaaagagccaccctctcctgcagggccagtcagagtgttcccaggaattatataggttggttccagcagaaacctggccaggctcccaggctcctcatctatggtgcatccagcagggccgctggcttcccagacagattcagtggcagtgggtctgggacagacttcactctcaccatcaccagactggagcctgaagattttgcaatgtattactgtcaccagtatgataggttaccgtacacttttggccaggggaccaagctggagatcaaac

SEQ ID NO: 100 - DNA, 325 bp, synthetic construct
  source 1..325: mol_type = other DNA
gaaattgtgttgacgcagtctccaggcaccctgtctttgtctccaggggaaagagccaccctctcctgcagggccactcagtatgttagcagcaatcacttagcctggtaccagcagaaacctggccaggctcccaggctcctcatgtatggtgcatccattagggccactggcatcccagacaggttcagtggcagtgggtctgggacagacttcactctcaccatcagcggactggagcctgaagactttgcagtttattactgtcaccagtatgccacctcaccgctcgctttcggcggagggaccagggtggagatcaaac

SEQ ID NO: 101 - DNA, 325 bp, synthetic construct
  source 1..325: mol_type = other DNA
gaaattgttatgacccagactccattctccctgtctttgtctccaggggaaagagccaccctctcctgcagggccagtcagagtgttagcaacaacttcttagcctggtaccagcagaaacctggccaggctcccaggctcctcatctatggtgcatccaccagggccactggcatcccagacaggttcggtggcagtgggtctgggacagacttcactctcaccatcggcagactggagcctgaagattttgcagtgtattactgtcaccagtatgctacctcaccccggacgttcggccaagggaccaaggtggaactcaaac

SEQ ID NO: 102 - DNA, 325 bp, synthetic construct
  source 1..325: mol_type = other DNA
gaaattgttatgacccagtctccagtctccctgtctttgtctccaggggaaagagccaccctctcctgcagggccagtcagagtgttagcagcagctacttagcctggtaccagcagaaacctggccaggctcccaggctcctcatctatgctgcatccagcagggccactggcatctcagataggttcagtggcagtgggtctgggacagacttcactctcaccatcaacagactggagcctgaagattttgtagtatattactgtcagcactatggtaactcaccctggacgttcggccaagggaccaaggtggaaatcaaac

SEQ ID NO: 103 - DNA, 325 bp, synthetic construct
  source 1..325: mol_type = other DNA
gaaattgtgttgacgcagtctccaggcaccctgtctttgtctccaggggaaagagccaccctctcctgcagggccagtcagactgttaccagcagctccttagcctggtaccagcagaaacctggccaggctcccaggctcctcatctatggtgcatccagcagggccactggcatcccagacaggttcagtggcagtgggtctgggacagtcttcactctcaccatcagcagactggagcctgaagattttgcagtgtattactgtcagcagtatggtatctcaccctggtcgttcggccaagggaccaaggtggaaatcaaac

SEQ ID NO: 104 - DNA, 325 bp, synthetic construct
  source 1..325: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcgtctatggtgcatccaccagggccactggcatcccagacaggttcagtggcagtgggtctgggacagacttcactctcaccatcagcagactggagcctgaagattttgcagtgtattactgtcaccagtatagtagctcattctggacgttcggccaagggaccaaggtggaaatcaaac

SEQ ID NO: 105 - DNA, 325 bp, synthetic construct
  source 1..325: mol_type = other DNA
gaaattgtgttgacgcagtctccagtctccctgtctttgtctccaggggaaagagccaccctctcctgcagggccagtcagagtgttagcagcaactacttagcctggtaccagctgaaacctggccgggctcccaggctcctcatctatggtgcatccagcagggcccctggcatcccagacaggttcagtggcagtgggtctgggacagacttcactctcaccatcagcagactggagcctgaagattttgcagtgtattactgtcagcagtatggtatctcatcctggacgttcggccaagggaccaaggtggaaatcaaac

SEQ ID NO: 106 - DNA, 322 bp, synthetic construct
  source 1..322: mol_type = other DNA
gaaattgtgttgacgcagtctccagtctccctgtctttgtctccaggggaaagagccaccctctcctgcagggccagtcagagtgttaccagcagctacttagcctggtaccagcagaaacctggccaggctcccaggctcctcatctatggtgcatccatcagggccactggcatcccagacaggttcagtggcagtgggtctgggacagacttcactctcaccatcagcagactggagcctgaagattttgcagtgtattactgtcagcagtatggtagctcacttttttttggccaggggaccaagctggagatcaaac

SEQ ID NO: 107 - DNA, 328 bp, synthetic construct
  source 1..328: mol_type = other DNA
gaaattgttatgacccagtctccagtctccctgtctttgtctccaggagacagagccaccctctcctgcagggccagtcagagtgttagcagcagatatatagcctggtaccagcacaaacctggccaggctcccaggctcctcatctatgatgcatccagcagggccaatggcatcccagacaggttcagtggcagtgggtctgggacagacttcactctcaccattagcagactggagcctgaagactttgcagtgtattactgtcagcagtatggtagttcacctctgtggacttttggccaggggaccaagctggagatcaaac

SEQ ID NO: 108 - DNA, 328 bp, synthetic construct
  source 1..328: mol_type = other DNA
gaaattgtgttgacgcagtctccaggcaccctgtctttgtctccaggggaaagagccaccctctcctgcagggccagtcagagtattagcagcggctacatagcctggtaccagcagaaacctggccaggctcccaggctcctcatctatggtgcatccagcagggccactggcatcccagacaggttcagtggcagtgggtctgggacagacttcactctcaccatcagcagtctggagcctgaagattttgcagtttattactgtcagcagtatggtagttcacctctgtggacttttggccaggggaccaagctggacatcaaac

SEQ ID NO: 109 - DNA, 334 bp, synthetic construct
  source 1..334: mol_type = other DNA
caggctgtgctgactcaggagccctcactgactgtgtccccaggagggacagtcactctcacctgtggctccagcactggagctgtcaccagtggtcattatacttactggttccagcagaggtctggccaagcccccaggacactgatttatgatacaaccaacaaacactcctggacccctgcccggttctcaggctccctccttgggggcaaagctgccctgaccctttcgggtgcgcagcctgaggatgaggctgactattactgcttgctctcatatagtgatgtaccgtcaggggtgttcggcggagggaccaagctgaccgtcctag

SEQ ID NO: 110 - DNA, 334 bp, synthetic construct
  source 1..334: mol_type = other DNA
caggctgtggtgactcaggagccctcactgactgtgtctccaggagggacagtcactctcacctgtggctccagcactggagctgtcaccagtggtcattatccctactggttccagcagaagcctggccaagcccccaggacactgatttatgatacaaccaacaaacactcctggacccctgcccggttctcaggctccctccttgggggcaaagctgccctgaccctttcgggtgcgctgcctgacgatgaggctgactattattgtttgctctcctatagtgatgctccgtcaggggtgttcggcggagggaccaagctgaccgtcctgg

SEQ ID NO: 111 - DNA, 325 bp, synthetic construct
  source 1..325: mol_type = other DNA
cagtctgtgctgactcagccaccctcagtgtcagtggccccaggaaagacggccatgattacttgtgggggtaacagctttggaggtaaaagtgtgcactggtaccagcagaagccaggccaggcccctgtgttggtcatctattataatagcgaccggccctcagggatccctgagcgattctctggctccaactctgggagctcggccaccctgactatcagcagggtcgaagccggggatgaggccgactattactgtcaggtgtgggatagtagtagtgctctcggggtgttcggcggagggaccaagctgaccgtcctcg

SEQ ID NO: 112 - DNA, 325 bp, synthetic construct
  source 1..325: mol_type = other DNA
tcctatgtgctgactcaggcaccctcactgtcagtggccccaggaaagacggccaggattacctgtgggggaaacaacattggaagtaaaagtgtgcactggtaccagcagaagccaggcctggcccctgtattggtcatctattatcatagcgaccggccctcagggatccctgagcgattctctggctccatctctgggaacacggccaccctgaccatcagcagggtcgaagccggggatgaggccgactattactgtcaggtgtgggatagtactaatgctctcggggtgttcggcggagggaccaagctgaccgtcctag

SEQ ID NO: 113 - DNA, 331 bp, synthetic construct
  source 1..331: mol_type = other DNA
cagtctgtgctgactcagcctgcctccgtgtctgggtctcctggacagtcgatcaccatctcctgcactggaaccagcagtgacgttggtggttatgactatgtctcctggtaccaacagtacccaggcaaagcccccaaactcatgatttatgatgtcagtcagcggccctcaggggtttctaatcgcttctctggctccaagtctggcaacacggcctccctgaccatctctgggctccaggctgaggacgaggctgattattactgcagctcatatacaagcaccaccactctggtattcggcggagggaccaagttgaccgtcctcg

SEQ ID NO: 114 - DNA, 331 bp, synthetic construct
  source 1..331: mol_type = other DNA
cagtctgtgctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaagcggccctcaggggtttctaatcgcttctctggctccaagtctggcaacacggcctccctgaccatctctgggctccaggctgaggacgaggctgattattactgcagttcatatacaaacagcaatacttttgtcttcggaactgggaccagggtcaccgtcctag

SEQ ID NO: 115 - DNA, 331 bp, synthetic construct
  source 1..331: mol_type = other DNA
cagtctgtgctgactcagcctgcctccgtgtctgggtctcctggacagtcgatcaccatctcctgcactggaaccagcagtgacgtgggtggttataactatgtctcctggtaccaacatttcccaggcaaagcccccaaactcattatttatgatgtcagtaagcggccctcaggggtttctaatcgtttctctggctccaagtctggcaacgcggcctccctgaccatctctgggctccaggctgaggacgaggctgattattactgcagctcatatacaaggagctacacttgtgtcttcggaactgggaccaaggtcgccgtcctag

SEQ ID NO: 116 - DNA, 331 bp, synthetic construct
  source 1..331: mol_type = other DNA
cagtctgtgctgactcagccaccctcagcgtctgggacccccgggcagagggtcaccatctcctgttccggaggcagctccaacatcggaagagattatgtatactggtaccagcaggtcacaggaacggcccccaaactcctcatctatcggaatgatcagcggcccgcaggggtccctgaccgaatctctggctccaagtctggcacctcagcgtccctggccatcagtgggctccggtccgaggatgaggctgattattactgttcatcatgggatgacagtctgaatggttgggtgttcggcggagggaccaagttgaccgtcctag

SEQ ID NO: 117 - DNA, 331 bp, synthetic construct
  source 1..331: mol_type = other DNA
cagtctgtgctgactcagccaccctcagcgtctgggacccccgggcagagggtcatcatctcttgttccggaagcagctccaacatcggaagagagactgtaaactggtaccagcaggtccctggaacggcccccaaactcctcatctctggtaataatcagcggcccgcaggagtccctgaccgattctctggctccaagtctggcacctcagcctccctggccatcagtgggctccaatctgaggatgaggctgattattactgtgcatcttgggatgacagtctgattggtccggtgttcggcggagggaccaagctgaccgtcctag

SEQ ID NO: 118 - DNA, 334 bp, synthetic construct
  source 1..334: mol_type = other DNA
cagtctgtgctgactcagccaccctcagcgtctgggaccctcgggcagagggtcaccatctcttgttctggaagcagctccaacatcggaagttattctgttaactggtaccagcagctcccaggaacggcccccaaactcctcatctacaaggataatcagcggccctcaggggtccctgaccgattctctggctccaggtctggcacctcagcctccctggccatcagtgggctccagtctgaggatgaggctgattattactgtgcggcatgggatgacagcctgaatggtcatgtggtgttcggcggagggaccaagctgaccgtcctag

SEQ ID NO: 119 - DNA, 334 bp, synthetic construct
  source 1..334: mol_type = other DNA
cagtctgtgctgactcagccaccctcagcgtctgggaccctcgggcagagggtcaccatctcttgttctggaagcagctccaacatcggaagttactctgttaactggtaccagcagctcccaggaacggcccccaaactcctcatctacaacgataatcagcggccctcaggggtccctgaccgattctctggctccaggtctggcacctcagcctccctggccatcagtgggctccagtctgaggatgaggctgactattactgtgcagcatgggatgacagcctgaatggtcatgtggtgttcggcggagggaccaagctgaccgtcctag

SEQ ID NO: 120 - DNA, 331 bp, synthetic construct
  source 1..331: mol_type = other DNA
cagtctgtgctgactcaggcaccctcagcgtctgggacccccggccagagggtcaccatctcttgttctggaagcaggtccaacatcggaagtaatactgtaaactggtaccagcagctcccaggaacggcccccaaactcctcatttatagtaatgatcagcggccctcaggggtccctgaccgattctctggctccaagtctggcacctcagcctccctggccatcagtgggctccagtctgaggatgaggctgattattactgtgcagcatgggatgacagcctgaatgagtggctgttcggcggagggaccaagctgaccgtcctag

SEQ ID NO: 121 - DNA, 331 bp, synthetic construct
  source 1..331: mol_type = other DNA
cagtctgtgctgactcagccaccctcagcgtctgggacccccgggcagagggtcaccatctcttgttctggaagcagctccaacatcggaaagtttactgcaaactggtaccagcagctcccaggaacggcccccaaactcctcatctatagtaataatcagcggccctcaggggtccctgaccgattctctggctccaagtctggcacctcagcctccctggccatcactgggctccagtctgaagatgaggctgattattactgtgcagcatgggatgacagcctgaatggtcgggtgttcggcggagggaccaagttgaccgtcctag

SEQ ID NO: 122 - DNA, 337 bp, synthetic construct
  source 1..337: mol_type = other DNA
cagtctgtgctgacccagccgccctcagtgtctggggccccagggcagagggtcaccatttcctgcattgggagcagctccaacatcggggcaggttatgatgtccactggtaccagcaacttcctggaagagcccccaaactcctcatccacggtaacgccaatcggccctcaggggtccctgaccgattctctggctccaagtctggcacctcagcctccctggccatcactggcctccaggctgaggatgaggctgattattactgccagtcctatgacaacagcctgagaggttcgaaggttttcggcggagggaccaaggtgaccgtcctga

SEQ ID NO: 123 - DNA, 334 bp, synthetic construct
  source 1..334: mol_type = other DNA
cagtctgtgctgactcagccgccctcggtgtctggggccccagggcagagggtcaccatttcctgcactgggagcagctccagcatcggggcaggttacgatctgcactggtaccagcaggttccaggaacagccccgagactcctcatctataataacaataatcggccctcaggggtccctgaccgattctctggctccaagtctggcacctcagcctccctggccatcactgggctccaggctgaggatgaggctgattattactgccagtcctatgacagcggcctgaatggttatgtcttcggaactgggaccagggtcaccgtcctgg

SEQ ID NO: 124 - protein, 8 aa, synthetic construct
GYTYISTS

SEQ ID NO: 125 - protein, 8 aa, synthetic construct
IKTDDGST

SEQ ID NO: 126 - protein, 8 aa, synthetic construct
GYTFISNY

SEQ ID NO: 127 - protein, 8 aa, synthetic construct
INPSSGST

SEQ ID NO: 128 - protein, 8 aa, synthetic construct
GGTYINYA

SEQ ID NO: 129 - protein, 8 aa, synthetic construct
MSPISNTP

SEQ ID NO: 130 - protein, 8 aa, synthetic construct
GGTFMNND

SEQ ID NO: 131 - protein, 8 aa, synthetic construct
SIAMSGTT

SEQ ID NO: 132 - protein, 8 aa, synthetic construct
GGTLRLSE

SEQ ID NO: 133 - protein, 8 aa, synthetic construct
INPVLGTA

SEQ ID NO: 134 - protein, 8 aa, synthetic construct
GVSFSNFA

SEQ ID NO: 135 - protein, 8 aa, synthetic construct
IIPFLGTT

SEQ ID NO: 136 - protein, 8 aa, synthetic construct
GGTFSSYG

SEQ ID NO: 137 - protein, 8 aa, synthetic construct
IIPTFGRA

SEQ ID NO: 138 - protein, 8 aa, synthetic construct
GGSFSNYG

SEQ ID NO: 139 - protein, 8 aa, synthetic construct
IIPTLRTP

SEQ ID NO: 140 - protein, 8 aa, synthetic construct
GGPVSSYG

SEQ ID NO: 141 - protein, 8 aa, synthetic construct
IIPVFGRV

SEQ ID NO: 142 - protein, 8 aa, synthetic construct
GDTFSGYS

SEQ ID NO: 143 - protein, 8 aa, synthetic construct
IIPLFDTA

SEQ ID NO: 144 - protein, 8 aa, synthetic construct
GYSFITYG

SEQ ID NO: 145 - protein, 8 aa, synthetic construct
ISVYSGNT

SEQ ID NO: 146 - protein, 8 aa, synthetic construct
GYIFTSHG

SEQ ID NO: 147 - protein, 8 aa, synthetic construct
ISAYSGNT

SEQ ID NO: 148 - protein, 8 aa, synthetic construct
GFSFDDYG

SEQ ID NO: 149 - protein, 8 aa, synthetic construct
INWKGDSI

SEQ ID NO: 150 - protein, 8 aa, synthetic construct
GFSYDDYD

SEQ ID NO: 151 - protein, 8 aa, synthetic construct
ITWTGSNS

SEQ ID NO: 152 - protein, 8 aa, synthetic construct
GFSFSNYA

SEQ ID NO: 153 - protein, 8 aa, synthetic construct
FRGSDGST

SEQ ID NO: 154 - protein, 8 aa, synthetic construct
GFTFSSYA

SEQ ID NO: 155 - protein, 8 aa, synthetic construct
ISGSGDIT

SEQ ID NO: 156 - protein, 8 aa, synthetic construct
GFAFSSSW

SEQ ID NO: 157 - protein, 8 aa, synthetic construct
VNYDGIST

SEQ ID NO: 158 - protein, 8 aa, synthetic construct
GFYFSGYS

SEQ ID NO: 159 - protein, 8 aa, synthetic construct
ISSSGRTI

SEQ ID NO: 160 - protein, 8 aa, synthetic construct
GGSFSGYY

SEQ ID NO: 161 - protein, 8 aa, synthetic construct
INHSSGST

SEQ ID NO: 162 - protein, 20 aa, synthetic construct
CVSPRLGHCRRGRCYETLDW

SEQ ID NO: 163 - protein, 20 aa, synthetic construct
CVSPRIAHCRGGRCYETLDW

SEQ ID NO: 164 - protein, 22 aa, synthetic construct
CARDLLKYCGGGNCHSLLVDPW

SEQ ID NO: 165 - protein, 21 aa, synthetic construct
CARDLIGFCRGGRCYSWFDPW

SEQ ID NO: 166 - protein, 21 aa, synthetic construct
CAREEIKYCRGSSCYGWFDPW

SEQ ID NO: 167 - protein, 22 aa, synthetic construct
CASGFCSGGSCYVNFIYGMDVW

SEQ ID NO: 168 - protein, 18 aa, synthetic construct
CAIKILPPRLLFYGMDVW

SEQ ID NO: 169 - protein, 20 aa, synthetic construct
CARDPGIVSTGIYYTGMDVW

SEQ ID NO: 170 - protein, 18 aa, synthetic construct
CAMKLEVPRLLHYGMDVW

SEQ ID NO: 171 - protein, 18 aa, synthetic construct
CARDPWIAVGGVGYFENW

SEQ ID NO: 172 - protein, 16 aa, synthetic construct
CARAGEVYGDYYFDYW

SEQ ID NO: 173 - protein, 13 aa, synthetic construct
CARVEQLLLGDFW

SEQ ID NO: 174 - protein, 22 aa, synthetic construct
CARGDITLGRGVYYSYHGLDVW

SEQ ID NO: 175 - protein, 23 aa, synthetic construct
CVRGSYCTSTSCALYDYRGLDVW

SEQ ID NO: 176 - protein, 22 aa, synthetic construct
CAKSPGMIRFAGVIVKPVFDSW

SEQ ID NO: 177 - protein, 14 aa, synthetic construct
CATSYLSRGPMGDW

SEQ ID NO: 178 - protein, 22 aa, synthetic construct
CARDQGLRHFDTYDYYYVLDVW

SEQ ID NO: 179 - protein, 18 aa, synthetic construct
CARDTGYYDILTDYFSRG

SEQ ID NO: 180 - protein, 8 aa, synthetic construct
GYTYISTS

SEQ ID NO: 181 - protein, 8 aa, synthetic construct
IKTDDGST

SEQ ID NO: 182 - protein, 20 aa, synthetic construct
CVSPRLGHCRRGRCYETLDW

SEQ ID NO: 183 - protein, 8 aa, synthetic construct
GYTFISNY

SEQ ID NO: 184 - protein, 8 aa, synthetic construct
INPSSGST

SEQ ID NO: 185 - protein, 20 aa, synthetic construct
CVSPRIAHCRGGRCYETLDW

SEQ ID NO: 186 - protein, 8 aa, synthetic construct
GGTYINYA

SEQ ID NO: 187 - protein, 8 aa, synthetic construct
MSPISNTP

SEQ ID NO: 188 - protein, 22 aa, synthetic construct
CARDLLKYCGGGNCHSLLVDPW

SEQ ID NO: 189 - protein, 8 aa, synthetic construct
GGTFMNND

SEQ ID NO: 190 - protein, 8 aa, synthetic construct
SIAMSGTT

SEQ ID NO: 191 - protein, 21 aa, synthetic construct
CARDLIGFCRGGRCYSWFDPW

SEQ ID NO: 192 - protein, 8 aa, synthetic construct
GGTLRLSE

SEQ ID NO: 193 - protein, 8 aa, synthetic construct
INPVLGTA

SEQ ID NO: 194 - protein, 21 aa, synthetic construct
CAREEIKYCRGSSCYGWFDPW

SEQ ID NO: 195 - protein, 8 aa, synthetic construct
GVSFSNFA

SEQ ID NO: 196 - protein, 8 aa, synthetic construct
IIPFLGTT

SEQ ID NO: 197 - protein, 22 aa, synthetic construct
CASGFCSGGSCYVNFIYGMDVW

SEQ ID NO: 198 - protein, 8 aa, synthetic construct
GGSFSNYG

SEQ ID NO: 199 - protein, 8 aa, synthetic construct
IIPTLRTP

SEQ ID NO: 200 - protein, 20 aa, synthetic construct
CARDPGIVSTGIYYTGMDVW

SEQ ID NO: 201 - protein, 8 aa, synthetic construct
GGPVSSYG

SEQ ID NO: 202 - protein, 8 aa, synthetic construct
IIPVFGRV

SEQ ID NO: 203 - protein, 18 aa, synthetic construct
CAMKLEVPRLLHYGMDVW

SEQ ID NO: 204 - protein, 8 aa, synthetic construct
GYSFITYG

SEQ ID NO: 205 - protein, 8 aa, synthetic construct
ISVYSGNT

SEQ ID NO: 206 - protein, 16 aa, synthetic construct
CARAGEVYGDYYFDYW

SEQ ID NO: 207 - protein, 15 aa, synthetic construct
CARVNDYGAYVFDLW

SEQ ID NO: 208 - protein, 16 aa, synthetic construct
CARAGEVYGDYYFDYW

SEQ ID NO: 209 - protein, 26 aa, synthetic construct
CAREGAGTVVLRANVGHSSHYGMDVW

SEQ ID NO: 210 - protein, 16 aa, synthetic construct
CAKEQSNYYGSGVDYW

SEQ ID NO: 211 - protein, 20 aa, synthetic construct
CARGQRNCSGGNCYSGLWAW

SEQ ID NO: 212 - protein, 16 aa, synthetic construct
CAREAQDYRGYRFDSW

SEQ ID NO: 213 - protein, 16 aa, synthetic construct
CAKEQSNYYGSGVDYW

SEQ ID NO: 214 - protein, 18 aa, synthetic construct
CARDPWIAVGGVGYFENW

SEQ ID NO: 215 - protein, 23 aa, synthetic construct
CVRGSYCTSTSCALYDYRGLDVW

SEQ ID NO: 216 - protein, 25 aa, synthetic construct
CARFLPPDYDLLTDYFDPPGAFDIW